(19) United States
(12) Patent Application Publication
(10) Pub. No.: US 2003/0149045 A1
(43) Pub. Date: Aug. 7, 2003

(54) Therapeutic compounds

(76) Inventor: M. Uckun Fatih, White Bear Lake, MN (US)

Correspondence Address: MERCHANT & GOULD PC, P.O. BOX 2903, MINNEAPOLIS, MN, 55402-0903, US

(21) Appl. No.: 10/211,045

(22) Filed: Aug. 2, 2002

Related U.S. Application Data
(63) Continuation-in-part of application No. 09/812,098, filed on Mar. 19, 2001, now Pat. No. 6,495,556.
Application No. 09/812,098 is a continuation of application No. 09/378,093, filed on Aug. 20, 1999, now Pat. No. 6,313,129.
(60) Provisional application No. 60/309,557, filed on Aug. 2, 2001.
(60) Provisional application No. 60/309,558, filed on Aug. 2, 2001.

(51) Int. Cl. A61K 31/525; A61K 31/517

(52) U.S. Cl. 514/251; 514/266.1; 514/266.3; 514/266.4

(57) ABSTRACT

The invention provides novel JAK-3 inhibitors that are useful for treating leukemia and lymphoma. The compounds are also useful to treat or prevent skin cancer, as well as sunburn and UVB-induced skin inflammation. In addition, the compounds of the present invention prevent the immunosuppressive effects of UVB radiation, and are useful to treat or prevent autoimmune diseases, inflammation, and transplant rejection. The invention also provides pharmaceutical compositions comprising compounds of the invention, as well as therapeutic methods for their use.

### PRIORITY OF INVENTION

This application is a continuation-in-part, claiming priority under 35 U.S.C. §120, of U.S. application Ser. No. 09/812,098, filed Mar. 19, 2001 which is a continuation of U.S. application Ser. No. 09/378,093 filed Aug. 20, 1999, now U.S. Pat. No. 6,313,129, and claims priority under 35 U.S.C. §119(e) from U.S. Provisional application No. 60/309,557 and from U.S. Provisional application No. 60/309,558, each filed Aug. 2, 2001, the disclosures of which are hereby incorporated by reference.

### BACKGROUND OF THE INVENTION

Bone marrow transplantation (BMT) has become one of the standard treatment modalities offered to high-risk leukemia patients. Very intensive “supralethal” myeloablative chemotherapy or radiochemotherapy regimens can be applied in the context of BMT with a curative intent to overcome the drug resistance of residual leukemia cells of certain leukemia patients who are unlikely to be cured by standard chemotherapy. In addition, leukemia patients undergoing allogeneic BMT may benefit from the graft-versus-leukemia (GVL) effect of the marrow allograft.

Graft-Versus-Host Disease (GVHD), a donor T-cell initiated highly complex pathologic condition that frequently follows allogeneic BMT, especially in the context of a major-HLA disparity, is associated with significant morbidity and mortality. Severe GVHD remains a major obstacle to a more successful outcome of allogeneic BMT using HLA-matched unrelated donors as well as partially HLA-mismatched related donors. Therefore, GVHD prophylaxis aimed at reducing the risk of severe GVHD is an integral component of all BMT programs. On the other hand, there is a widely accepted notion in the BMT community that leukemia patients who develop GVHD after BMT have a reduced risk of relapse indicating that alloreactive T cells participating in GVHD are major contributors of the GVL function of the marrow allografts. In certain subsets of leukemia patients who relapsed after BMT (e.g. chronic myelogenous leukemia patients), infusions of donor T-cells resulted in remissions, thereby providing direct evidence that host leukemia cells can be killed by alloreactive T-cells. Therefore, several groups involved in the treatment of leukemia patients are currently exploring alternative methods of inducing mixed chimerism with non-myeloablative conditioning regimens followed by donor lymphocyte infusions to achieve a GVL effect without severe GVHD.

Contemporary methods for GVHD prophylaxis, including ex vivo T-cell depletion of marrow grafts, use of positively selected CD34⁺ hematopoietic precursor cells, and systemic immunosuppression are associated with an increased risk of relapse in leukemia patients undergoing BMT, which has generally been attributed to loss of the GVL function of the marrow allografts. Hence, novel anti-GVHD agents which spare the GVL function of the marrow allografts are urgently needed for effective prevention of GVHD after BMT without facilitating the recurrence of leukemia.

Signal Transducers and Activators of Transcription (STATs) are a family of DNA binding proteins that reside in the cytoplasm until they are activated by tyrosine phosphorylation. This phosphorylation event is catalyzed by members of the Janus family of tyrosine kinases, including JAK3 (Ihle, J. N. Adv. Immunol. 60: 1-35, 1995; Witthuhn, B. A., et al., Leukemia and Lymphoma. 32: 289-297, 1999).

The dual role of STATs as signaling molecules and transcription factors is reflected in their structure. All STAT proteins contain a DNA binding domain, an SH2 domain, and a transactivation domain necessary for transcriptional induction. In unstimulated cells, latent forms of STATs are predominantly localized in the cytoplasm. Ligand binding induces STAT proteins to bind with their SH2 domains to the tyrosine phosphorylated motifs in the intracellular domains of various transmembrane cell surface receptors (Horvath, C. M. and Darnell, J. E., Curr. Opin. Cell. Biol. 9(2): 233-239., 1997; Levy, D. E., Cytokine Growth Factor Rev. 8(1): 81-90, 1997).

Once STATs are bound to receptors, the receptor-associated Janus kinases (JAKs) phosphorylate STATs on a single tyrosine residue located near the SH2 domain. Two STATs then dimerize through specific reciprocal SH2-phosphotyrosine interactions. The dimerized STAT proteins can also form complexes with other DNA-binding proteins. The STAT dimers/complexes next translocate to the nucleus and utilize their DNA binding domain to interact with DNA response elements in promoters of target genes (Demoulin, J. B., et al., Mol. Cell. Biol. 16: 4710-6, 1996). STATs then interact directly or indirectly, via their transactivation domain, with components of the RNA polymerase II complex to activate transcription of target genes. Different ligands employ specific JAK and STAT family members, thus utilization of this pathway mandates specificity in signaling cascades and contributes to a diverse array of cellular responses.

JAKs, including JAK-3, are abundantly expressed in primary leukemic cells from children with acute lymphoblastic leukemia (ALL), the most common form of childhood cancer, and recent studies have correlated STAT activation in ALL cells with signals regulating apoptosis (Demoulin, J. B., et al., Mol. Cell. Biol. 16: 4710-6, 1996; Jurlander, J., et al., Blood. 89: 4146-52, 1997; Kaneko, S., Suzuki, et al., Clin. Exp. Immun. 109: 185-193, 1997; and Nakamura, N., et al., J. Biol. Chem. 271: 19483-8, 1996).

Thus, JAK-3 is an important enzyme that plays an essential role in the function of lymphocytes, macrophages, and mast cells. Compounds which inhibit JAK-3 would be expected to be useful for treating or preventing diseases or conditions wherein the function of lymphocytes, macrophages, or mast cells is implicated, such as, leukemia, lymphoma, transplant rejection (e.g. pancreas islet transplant rejection, bone marrow transplant applications (e.g. graft-versus-host disease), autoimmune diseases (e.g. diabetes), and inflammation (e.g. asthma, inflammation associated with sun burn, and skin cancer). A continuing need exists for compounds and methods that are useful for the treatment and/or prevention of such conditions and diseases.

### SUMMARY OF THE INVENTION

The present invention provides JAK-3 inhibiting compounds that are nontoxic in the administered dosage range. The JAK-3 inhibitors of the invention are useful for treating leukemia and lymphoma. The compounds are also useful to prevent skin cancer, as well as to treat or prevent sunburn and UVB-induced skin inflammation. In addition, the compounds of the present invention prevent the immunosuppressive effects of UVB radiation, and are useful to treat or prevent autoimmune diseases, inflammation, and transplant rejection.

The present invention provides a therapeutic method for treating leukemia or lymphoma comprising administering to the mammal in need thereof an effective amount of a JAK-3 inhibitor.

The present invention also provides a therapeutic method for preventing or reducing UV B radiation-induced inflammatory response in a mammal comprising administering to the mammal in need thereof an effective amount of a JAK-3 inhibitor.

The present invention also provides a therapeutic method for inhibiting the release of prostaglandin E₂ in a mammal comprising administering to the mammal in need thereof an effective amount of a JAK-3 inhibitor.

The present invention also provides a therapeutic method for preventing or reducing UVB-induced skin edema or vascular permeability changes in a mammal comprising administering to the mammal in need thereof an effective amount of a JAK-3 inhibitor.

The present invention also provides a therapeutic method for preventing or reducing UV B radiation-induced damage to epithelial cells or mutation frequency in skin in a mammal comprising administering to the mammal in need thereof an effective amount of a JAK-3 inhibitor.

The present invention also provides a therapeutic method for protecting a mammal from tumorigenic effects of UVB light comprising administering to the mammal in need thereof an effective amount of a JAK-3 inhibitor.

Representative JAK-3 inhibitors of the invention have also been found to exhibit significant anti-proliferative activity against T-cells, and have been found to inhibit IL-2 dependent cell proliferation. Thus, the compounds can be used to treat or prevent transplant complications (e.g. rejection of a donor organ transplant by the host immune system), and complications associated with bone marrow transplantation such as graft versus host disease (GVHD).

In addition, the compounds of the invention are effective in treating and preventing autoimmune diseases, such as insulin dependent diabetes. The compounds are also effective in treating airway inflammation (asthma).

In addition, the compounds of the invention are effective in treating and preventing GVHD, and in treating and preventing GVHD without compromising the graft-versus leukemia (GVL) function of the bone marrow transplant (BMT) graft.

In addition, the compounds of the invention are effective in combination with other compounds, such as methotrexate, in treating and preventing GVHD, and in treating and preventing GVHD without compromising the GVL function of the BMT graft.

Accordingly, the invention also provides a therapeutic method for treating (or preventing) leukemia, transplant rejection, graft-verses host disease, inflammation, asthma, autoimmune diseases including diabetes, and inflammation related cancer development in the skin, comprising administering to the mammal in need thereof an effective amount of a compound of formula I.

The invention also provides novel compounds of formula I disclosed herein as well as pharmaceutical compositions comprising compounds of formula I.

A specific JAK-3 inhibitor useful in the medicaments and methods of the invention is a compound of formula I:

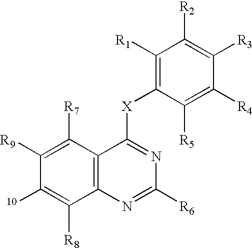

wherein:

X is H N, R₁₁ N, S, O, CH₂, or R₁₁CH;

R₁₁ is hydrogen, (C₁-C₄)alkyl, or (C₁-C₄)alkanoyl;

R₁-R₈ are each independently hydrogen, hydroxy, mercapto, amino, nitro, (C₁-C₄)alkyl, (C₁-C₄)alkoxy, (C₁-C₄)alkylthio, or halo; wherein two adjacent groups of R₁-R₅ together with the phenyl ring to which they are attached may optionally form a fused ring, for example forming a naphthyl or a tetrahydronaphthyl ring; and further wherein the ring formed by the two adjacent groups of R₁-R₅ may optionally be substituted by 1, 2, 3, or 4 hydroxy, mercapto, amino, nitro, (C₁-C₄)alkyl, (C₁-C₄)alkoxy, (C₁-C₄)alkylthio, or halo; and R₉ and R₁₀ are each independently hydrogen, (C₁-C₄)alkyl, (C₁-C₄)alkoxy, halo, or (C₁-C₄)alkanoyl; or R₉ and R₁₀ together are methylenedioxy; or a pharmaceutically acceptable salt thereof. Preferably, at least one of R₂ and R₃ is hydroxy. More preferably, at least one of R₂ and R₃ is hydroxy, and one of R₁ to R₅ is halo.

The present invention also provides compositions containing a compound of formula I and methotrexate; and pharmaceutical compositions containing a compound of formula I, methotrexate, and a pharmaceutically accpetable carrier.

### BRIEF DESCRIPTION OF THE DRAWINGS

FIG. 1. Illustrates the synthesis of representative compounds of formula I from compound 5.

FIG. 2. [A] Model of JAK3 showing molecular surface of protein (blue), and catalytic (ATP binding) site (yellow). [B] Ribbon representation (Ca backbone) of the homology model of the JAK3 kinase domain. The WHI-P131 molecule is shown as a space-filling model in the catalytic site of JAK3. [C] Close-up view of catalytic site of JAK3 model with docked quinazoline inhibitor WHI-P131 (green). Residues and inhibitor are shown as space-filling atoms. The solvent-exposed opening of the catalytic site has dimensions to allow a relatively planar inhibitor to enter and bind to JAK3. The opening of the pocket is defined by residues Pro906, Ser907, Gly908, Asp912, Arg953, Gly829, Leu828, and Tyr904 (blue residues). The far wall deep inside the pocket is lined with Leu905 (Cα backbone), Glu903, Met902, Lys905, and Asp967 (pink residues), and the floor of the pocket is lined by Leu905 (side chain), Val884, Leu956, and Ala966 (yellow residues). Residues defining the roof of the pocket include Leu828, Gly829, Lys830, and Gly831 (uppermost blue residues). Prepared using InsightII program.

FIG. 3. [A] Model of unoccupied space in the catalytic (ATP binding) site of a JAK3 homology model. Shown in green is the binding site for ATP and the most likely binding site for dimethoxyquinazoline inhibitors. The green kinase active site region represents a total volume of approximately 530 Å. Modeling studies showed that an inhibitor or a portion of an inhibitor with significant binding to this region would occupy a volume less than 530 Å³ and have molecular dimensions compatible with the shape of the binding site region. Other regions near the binding site which show measurable unoccupied volume are shown in royal blue, pink, yellow, and light blue. These binding regions are either unavailable to inhibitor molecules (royal blue) or represent regions just large enough to occupy solvent molecules (pink, yellow, light blue). A model of WHI-P131 docked into the catalytic site is shown in white, superimposed on the green region. [B] Model of the catalytic site of JAK3 with quinazolines WHI-P131 (multicolor), WHI-P132 (pink), and WHI-P154 (yellow). Each compound fits into the binding site but WHI-P132 (shown to be inactive against JAK3 in biological assays) lacks an OH group that is in a location to bind with Asp967. WHI-P131 and WHI-P154, with OH groups at the C4′ position of the phenyl ring, are able to form a favorable interaction with Asp967 of JAK3, which may contribute to their enhanced inhibition activity. [C] Features of dimethoxyquinazoline derivatives which are predicted to aid binding to JAK3 catalytic site.

FIG. 4. [A] Structural comparison of nonconserved residues in the catalytic sites of 5 different protein tyrosine kinases: JAK3 (pink), BTK (red), SYK (light blue), IRK (dark blue), and HCK (yellow). Residues within 5 Å of the docked JAK3 inhibitor, WHI-P131 (white), are shown as rod-shaped side chains. The C alpha backbone of JAK3 is shown as a thin pink line, for perspective. Regions A to F correspond to areas containing nonconserved residues in the catalytic site (see B and Results and Discussion). Crystal structure coordinates of HCK and IRK, and homology models of JAK3, BTK, and SYK were used for the structural analysis. [B] Nonconserved residues in the catalytic sites of 8 different protein tyrosine kinases. Regions A-F refer to locations in the catalytic site which are illustrated in A.

FIG. 5. Effects of WHI-P131 on the Tyrosine Kinase Activity of JAK3. [A]-[D]. JAK3, JAK1, and JAK2 immunoprecipitated from Sf21 insect ovary cells transfected with the appropriate baculovirus expression vectors were treated with WHI-P131, then subjected to in vitro kinase assays as described in Methods. The enzymatic activity of JAKs was determined by measuring autophosphorylation in a 10 min kinase assay, as described in Methods. The kinase activity (KA) levels were expressed as percentage of baseline activity (%CON). [E] EMSAs of 32Dc22-IL-2Rβ cells. WHI-P131 (10 μg/ml=33.6 μM) and WHI-P154 (10 μg/ml=26.6 μM) (but not WHI-P132; 10 μg/ml=33.6 μM) inhibited IL-2 triggered JAK-3-dependent STAT activation but not IL-3-triggered JAK-1/JAK-2-dependent STAT activation in 32Dc11-IL-2Rβ cells.

FIG. 6. Specificity of WHI-P1131. JAK3, SYK, and BTK immunoprecipitated from Sf21 insect ovary cells transfected with the appropriate baculovirus expression vectors, LYN immunoprecipitated from NALM-6 human B-lineage ALL cells, and IRK immunoprecipitated from HepG2 hepatoma cells were treated with WHI-P131, then subjected to in vitro kinase assays as described in Methods.

FIG. 7. WHI-P131 Depolarizes Mitochondrial Membranes in a Concentration-Dependent Fashion Without Affecting the Mitochondrial Mass. NALM-6 human leukemic cells were incubated with indicated concentrations of WHI-P131 for 48 h, stained with DiIC1 to assess the mitochondrial membrane potential (Δψm) or NAO to detect the mitochondrial mass and then analyzed with cell sorter equipped with HeNe laser. WHI-P131 caused a progressive increase in depolarized mitochondria (as indicated by M1 in A) with increasing concentrations. At similar concentrations no significant change in mitochondrial mass [B] was detected. [C]: Cells were stained with JC-1 for simultaneous analysis of mitochondrial mass (green fluorescence) and mitochondrial transmembrane potential (red/orange fluorescence). Untreated NALM-6 cells [D.1] as well-as NALM-6 cells treated with 50 μM of WHI-P131 for 24 hr [D.2] were incubated with JC-1 and analyzed by confocal laser scanning microscopy. Mitochondria of control cells showed a higher membrane potential (Δψm), as indicated by brighter JC-1 red fluorescence. Treatment of cells with WHI-P131 reduced mitochondrial membrane Δψm as indicated by a substantial decrease in JC-1 red fluorescence.

FIG. 8. WHI-P131 Induces Apoptosis in NALM-6 Leukemia Cells. [A]: Cells were incubated with 10μM-500 μM WHI-P131 for 24 hours and then processed for in situ apoptosis assays. The percentage of apoptotic cells was determined by examining an average of 1380 cells/10 fields/sample. Data points represent the mean from duplicate counts obtained in two independent experiments. [B.1], [B.2]: NALM-6 cells were incubated with 100 μM of WHI-P131 for 48 hr, processed for the in situ apoptosis assay and analyzed with a laser scanning confocal microscope. When compared with controls treated with DMSO (0.1%) [B.1], several of the cells incubated with WHI-P131 [B.2] showed apoptotic nuclei (yellow fluorescence). Red fluorescence represents nuclei stained with propidium iodide.

FIG. 9. WHI-P131 Induces Apoptotic DNA Fragmentation in Human Leukemia Cells. DNA from Triton-X-100 lysates of control and drug treated cells was analyzed for fragmentation, as described. [A] NALM-6 human B-lineage ALL cells were treated for 24 hours at 37° C. with the listed dimethoxyquinazoline compounds at 1, 3, or 10 μM final concentrations. WHI-P131 was used as the lead JAK3 inhibitory compound and all other compounds were included as controls which lacked JAK3 inhibitory activity. [B] LC 1; 19 human B-lineage ALL cells and the control cell lines SQ20B and M24-MET were treated for 24 hours at 37° C. with WHI-P131 at 1 or 3 μM final concentrations.

FIG. 10. Inhibitory effect of compound 6 on UVB-induced skin thickness in skh-1 mice. Female skh-1 mice were treated topically with 1 mg/cm² of compound 6 prior to each UVB light exposure with 35 mj/cm². Skinfold thickness of each mouse was recorded twice weekly and the average of the two measurements was used in calculations. Data represent mean±SEM (n=5-14). * P≦0.05 and ** P≦0.005 as compared to vehicle treated control.

FIG. 11. Inhibitory effect of compound 6 on the average number of lesions per mouse. Female skh-1 mice were treated topically with 1 mg/cm² of compound 6 prior to each UVB light exposure with 35 mj/cm². The number of lesions was recorded twice weekly and the average of the two measurements was used in calculations. Data represent mean±SEM (n=5-14). * P<0.05 as compared to vehicle treated control.

FIG. 12. Inhibitory effect of Compound 6 on the average lesion volume per mouse. Female skh-1 mice were treated topically with 1 mg/cm² of compound 6 prior to each UVB light exposure with 35 mj/cm². The lesions greater than or equal to 1 mm in diameter were measured and recorded twice weekly and an average of the two measurements was used in calculations. Lesion volume was calculated using the formula described in Material and Methods. Data represent mean±SEM (n=5-14). * P<0.05 as compared to vehicle treated control.

FIG. 13. Morphological appearance of dorsal surface of mice after 20 weeks of UVB irradiation. Female skh-1 mice were treated topically with 1 mg/cm² of compound 6 prior to each UVB light exposure with 35 mj/cm². Mice were irradiated three times per week for a total of 20 weeks. At 20 weeks the mice were anaesthetized, and a picture of their dorsal surface was taken. Panel A, Unirradiated and vehicle treated control. Panel B, UVB irradiated and vehicle-treated mouse. Panel C, UVB irradiated and compound 6-treated mouse. Magnification 2×.

FIG. 14. Compound 6 inhibits UVB-induced PGE₂ synthesis. HaCaT cell cultures were either irradiated with UVB (25 mj/cm²) or sham irradiated. COMPOUND 6 (3-30 (M) was added 60 min prior to irradiation and was readministered after UVB exposure. Cumulative PGE₂ released during subsequent 6 h incubation was determined by EIA. Data represent mean (SEM (n=3).

FIG. 15. Compound 6 inhibits EGF-stimulated PGE₂ release in epidermal cells. 50 ng/ml EGF was used to stimulate confluent HaCaT cell cultures both in the absence and presence of compound 6 and the cells were incubated for 6 h at 37 (C. Following incubation the supernatant was collected, and PGE₂ released during incubation was determined by EIA. Data represent mean (SEM (n=5).

FIG. 16. Effect of Compound 6 on skinfold thickness of skh-1 mice following UVB light injury. Skh-1 mice were pretreated with compound 6 (16 mg/kg; i.p. bolus injection) for 2 days. On the day of UVB irradiation the mice were anaesthetized and painted with 1.5 mg/cm² compound 6 on dorsal surface 15 minutes before UVB exposure, and irradiated with UVB light (250 mj/cm²). The skinfold thickness was measured on day 1 through 5 post-irradiation. The data are expressed as mean (SEM (n=5-34).

FIG. 17. Inhibition of UVB-induced plasma exudation in skin of skh-1 mice. Plasma exudation was evaluated at the times indicated after UVB exposure (250 mj/cm²) by measuring the absorbance of Evans blue in skin extracts following the method described in Materials and Method. Data represent mean (SEM (n=5-17).

FIG. 18. Effect of compound 6 on skin morphology at day 4 post UVB-irradiation. Skh-1 mice were pretreated with compound 6 (16 mg/kg; i.p. bolus injection) for 2 days, painted with 1.5 mg/cm² compound 6 on dorsal surface 15 minutes before UVB exposure, and irradiated with UVB light (250 mj/cm²). On day 4 mice were anaesthetized and a picture of their dorsal surface was taken. Magnification 2×.

FIG. 19. Inhibition of UVB-induced histological changes in skin of skh-1 mice by compound 6. Mice were treated with drug and exposed to UVB (250 mj/cm²) following the same procedure as described in Figure legend 3. At 48 h post-irradiation mice were sacrificed, skin was biopsied and paraffin sections of the tissue were stained with hematoxylin and eosin. (a) control, (b) UVB (250 mj/cm²), and (c) COMPOUND 6+UVB (250 mj/cm²). Magnification 40×.

FIG. 20. Apoptosis of epidermal cells in UVB irradiated skin in skh-1 mice and its inhibition by compound 6. Skin biopsies from the irradiated and sham irradiated mice with or without drug treatment were taken out at 48 h post UVB-irradiation and paraffin sections obtained were stained for apoptotic cells. The figure shows images captured by confocal microscope using 60× magnification. Green stain in the picture shows apoptotic cells.

FIG. 21. Dose-dependent suppression of MLR (A), PHA-induced (B) and ConA-induced (C) proliferation of splenocytes by WHI-P131. WHI-P131 was added in the concentration of 0.1, 1.0, 10, and 50 μg/mL during the 5-day culture (MLR). or 3-day culture period (PHA and ConA). Proliferation was measured by WST-1 colorimetric assay. Results are presented as mean O.D.±SEM of 3-7 separate experiments. Statistical differences between the groups analyzed by Student's t-test.

FIG. 22. Flow cytometry analysis of the percentage of apoptotic splenocytes (TUNEL-positive) obtained after the 24-h-culture period with addition of 0.1, 1, 10 and 100 μg/ml of WHI-P131. Results are presented as mean±SEM. Statistical differences between the groups analyzed by Student's t-test.

FIG. 23. The in vivo prophylactic effect of WHI-P131 administration on GVHD induced across the major histocompatibility barrier in C57BL6 (H-2^{b}) recipients with BALB/c (H-2^{d}) BM/splenocyte grafts. Irradiated (7.5 Gy) recipients were given BM and splenocytes (25×10⁶ of each). Some recipients received syngeneic BM, while others were treated daily with 25 mg/kg of WHI-P131, 50 mg/kg of WHI-P132, 3 mg/kg of Cyclosporine A, 10 mg/m² of Methotrexate or vehicle control, as described in Material and Method section. Differences in survival between the groups were analyzed by life-table analysis, Mantel-Cox test.

FIG. 24. The in vivo prophylactic effect of administration of drug combinations presented in FIG. 23 on GVHD induced across the major histocompatibility barrier in C57BL6 (H-2^{b}) recipients with BALB/c (H-2^{d}) BM/splenocyte grafts. WHI-P131 (25 mg/kg), cyclosporine A (3 mg/kg) or combination of cyclosporine A and WHI-P131 (A); were administered i.p. Differences in survival between the groups were analyzed by life-table analysis, Mantel-Cox test.

FIG. 25. The in vivo prophylactic effect of administration of drug combinations presented in FIG. 23 on GVHD induced across the major histocompatibility barrier in C57BL6 (H-2^{b}) recipients with BALB/c (H-2^{d}) BM/splenocyte grafts. WHI-P131 (25 mg/kg), and WHI-P131 and methotrexate (10 mg/m²) or combination of methotrexate and WHI-P131 were administered i.p. Differences in survival between the groups were analyzed by life-table analysis, Mantel-Cox test.

FIG. 26. shows the effects of compound 6 (60 mg/kg/day) on GVHD development.

FIG. 27. Cumulative diabetes incidence (A) in LDSTZ-treated Jak3-deficient and wild-type (WT) males studied during the experimental period of 25 days post administration of first STZ injection; statistical difference obtained by life table analysis.

FIG. 28. Blood glucose level (mg/dl) (B) in LDSTZ-treated Jak3-deficient and wild-type (WT) males studied during the experimental period of 25 days post administration of first STZ injection; statistical difference obtained by ANOVA.

FIG. 29. Cumulative diabetes incidence (A) in LDSTZ-treated C57BL/6 males studied during the experimental period of 25 days post administration of first STZ injection; statistical difference obtained by life table analysis (Mantel-Cox test).

FIG. 30. Blood glucose level (mg/dl) in LDSTZ-treated C57BL/6 males studied during the experimental period of 25 days post administration of first STZ injection; statistical difference obtained by ANOVA.

FIG. 31. Diabetes incidence in NOD females treated with 20 and 50 mg/kg of WHI-P131 daily from 5 to 25 wk of age (A) statistically significant differences between WHI-P131-treated and control mice were obtained by life table analysis (Mantel-Cox test).

FIG. 32. Diabetes incidence in NOD females treated with 100 mg/kg of WHI-P131 from 5 or 10 to 25 wk of age; statistically significant differences between WHI-P131-treated and control mice were obtained by life table analysis (Mantel-Cox test).

FIG. 33. IPGTT test performed in C57BL/6 females (20-wk-old), non-diabetic control NOD females (25-wk-old) and NOD females (25-wk-old) treated for 15 or 20 weeks with 100 mg/kg of WHI-P131; statistical differences obtained by Student's t-test: *, **, ***, **** p<0.05, 0.01, 0.005 and 0.0001, respectively, compared to NOD vehicle-control group.

FIG. 34. Delayed adoptive transfer of diabetes into NOD-scid females by WHI-P131 treatment. NOD-scid females were transferred with 10×10⁶ diabetic splenocytes and treated daily with 50 mg/kg of WHI-P131 i.p. till diabetes onset. Statistically significant difference obtained by life table analysis (Mantel-Cox test).

FIG. 35. Jak3-1 mice do not reject islet allograft (A); hematoxilin and eosin (B) and immunostaining for insulin (C) of allogeneic islet graft transplanted under the kidney capsule of diabetic Jak3^{-/-} recipient sacrificed on day 100 post transplantation. Data representative of >30 sections/graft and 6 mice/group; ×10.

FIG. 36. WHI-P131 suppresses MLR reaction. Responder splenocytes (4×10⁶/ml) were mixed with stimulator splenocytes (1.6×10⁶/ml) and drugs were added in the concentration of 0.1, 1, 10 and 50 μg/ml during the 5-day-culture period. Proliferation was measured by WST-1 colorimetric assay. Results are presented as mean O.D.±SEM of 6 separate experiments; p=0.0095 compared to proliferation of control cells not-exposed to the WHI-P131.

FIG. 37. Flow cytometry analysis of apoptotic splenocytes (TUNEL-positive). TUNEL staining was performed after 20 h incubation period of splenocytes (1.5×10⁶) with 0.1, 1, 10 and 100 μg/ml of WHI-P131. Results are presented as mean±SEM. Statistical differences between the groups analyzed by Student's t-test.

FIG. 38. Effect of WHI-P131-(50 and 75 mg/kg), WHI-P132 (50 mg/kg) and cyclosporine A-treatment (20 mg/kg) on islet allograft survival; p values obtained by life table analysis (Mantel-Cox test).

FIG. 39. Flow cytometry analysis of C57BL/6 splenocytes after the treatment with 130 mg/kg of WHI-P131 for 10 days. Results are presented as mean±SEM. Statistical differences between the groups analyzed by Student's t-test.

FIG. 40. Non-fasting blood glucose (mg/dl) in syngeneic islet transplant recipients treated with 50 mg/kg/day of WHI-P131 or vehicle control during the period of 180 days post transplantation (A). Results are presented as mean±SD.

FIG. 41. IPGTT of the same recipients in FIG. 39 and non-transplanted control mice performed on day 70 (B) post transplantation after the fasting period of 8 hours. Results are presented as mean±SEM (B, C).

FIG. 42. IPGT™ of the same recipients in FIG. 39 and non-transplanted control mice performed on day 180 (C) post transplantation after the fasting period of 8 hours. Results are presented as mean±SEM.

FIG. 43. Abrogation of Proliferative Allo-Antigen and Mitogen Responses of Murine Splenocytes by the JAK3 Inhibitor WHI-P131. The effects of WHI-P131 on allo-antigen and mitogen responses of JAK3^{+/+} splenocytes from WT C57BL/6 mice were examined in MLR [A], PHA [B] and Con A [C] assays, as described. WHI-P131 was applied at concentrations of 0 (vehicle alone), 0.1, 1, 10 and 50 μg/ml during the 5-day (MLR) or 3-day culture period (PHA and ConA assays). Proliferation was measured using the colorimetric WST-1 assay. Results are presented as mean O.D. (±SEM) values from 3-6 independent experiments. Statistical differences between the WHI-P131 treatment groups and vehicle treated control groups were analyzed by Student's t-tests: *, P<0.05; **, P<0.0005, and *** P<0.0001.

FIG. 44. Anti-GVHD Activity of the JAK3 Inhibitor WHI-P131. [A. 1 & A.2] Histopathologic examination of the liver from the representative vehicle-treated control C57BL/6 (H-2^{b}) mouse transplanted with a BALB/c (H-2^{d}) BM/S graft, sacrificed on day 30 post-BMT, revealed a severe, predominantly lymphocytic, inflammatory reaction around the portal area with destruction of bile ducts. [A.3] Skin GVHD with focal pyknotic cells and vacuolation in the basal cell layer of the epidermis in skin of the same mouse. [B.1 & B.2] Histopathologic examination of the liver from the representative WHI-P131-treated C57BL/6 (H-2^{b}) mouse transplanted with a BALB/c (H-2^{d}) BM/S graft, sacrificed on day 30 post-BMT, revealed a mild lymphocytic infiltration of the portal area without a bile duct infiltration and destruction. [B.3] Normal appearing skin in the same mouse.

FIG. 45. Effects of the JAK3 Inhibitor WHI-P131 in Combination with Methotrexate on the Post-BMT Survival Outcome in A Murine Model of Acute GVHD. Irradiated (7.5 Gy) C57BL6 (H-2^{b}) recipients were given BM and splenocytes (25×10⁶ of each) from BALB/c (H-2^{d}) mice. Some recipients were transplanted with syngeneic BM/S grafts (Syngeneic). WHI-P131 was administered i.p. at a dose level of 60 mg/kg/day in 3 divided doses from day 0 to day 85. Methotrexate (MTX) was used at a dose level of 10 mg/m²/day once daily and administered i.p. on days 1, 3, 6 and 11 post BMT. *, P<0.0001 (Vehicle controls vs. WHI-P131, MTX, or WHI-P131+MTX treatment groups, log-rank test). See Table 7 for details of the life table analysis.

FIG. 46. WHI-P131 Does Not Exhibit Antileukemic Activity Against BCL-1 Leukemia Cells. Mice were treated with WHI-P131 following inoculation with 1×10⁶ BCL-1 leukemia cells. [A] BALB/c mice. BALB/c females were injected by 1×10⁶ BCL-1 cells and treated with WHI-P131 in a dose of 75 mg/kg/day (divided in 3 doses) and 150 mg/kg/day (divided in 3 doses) from the day of BCL-1 injection until the day of death. [B] F1 mice undergoing syngeneic BMT. Syngeneic BMT recipients were injected with 5×10⁶ of leukemia/lymphoma BCL-1 cells on day 0 and subjected to the WHI-P131 or vehicle alone treatment as described above.

FIG. 47. Effects of the JAK3 Inhibitor WHI-P131 on the Post-BMT Survival Outcome in a Murine GVHD Model [A.] An ill-appearing representative vehicle-treated control F1 (H-2^{d/b}) mouse transplanted with a B6 (H-2^{b}) BM/S graft (GVHD model), photographed on day 23 post-BMT. Clinical signs of GVHD in this mouse included >20% weight loss, hunching, ruffled fur and alopecia. [B] A healthy appearing representative WHI-P131-treated F1 (H-2^{d/b}) recipient transplanted with a B6(H-2^{b}) BM/S graft (Allo BMT->WHI-P131 group), photographed on day 65 post-BMT. [C] Irradiated (9.5 Gy) (BALB/cJx C57BL/6J)F1 (H-2^{d/b}) recipients were given BM/S graft (25×10⁶ cells of each) from syngeneic (syngeneic BMT) or allogeneic C57BL/6 (H-2^{b}) donors (allo BMT). Some mice were not transplanted after irradiation (TBI). Allotransplanted mice were administered with WHI-P131 daily (50 mg/kg/day in two divided doses) from day 0 until day 60 post BMT. Controls were treated with vehicle alone without added WHI-P131.

FIG. 48. Effects of the JAK3 Inhibitor WHI-P131 on the Post-BMT Survival Outcome in a Murine GVL Model. Irradiated (9.5 Gy) (BALB/cJx C57BL/6J)F1 (H-2^{d/b}) recipients were given BM/S graft (25×10⁶ cells of each) from syngeneic (Syngeneic BMT) or allogeneic C57BL/6 (H-2^{b}) donors (allo BMT). BMT recipients (GVL model) were injected with 5×10⁶ of leukemia/lymphoma BCL-1 cells on day 0. Recipients were treated with WHI-P131 (50 mg/kg/day in two divided doses) or vehicle (controls) alone daily from day 0 until day 60 post BMT. As there were no differences in survival rate of syngeneic BMT recipients treated with vehicle (N=22) or with WHI-P131 (N=13) (please, see Table 1), they were summarized and presented as Syngeneic BMT+BCl−1 group (N=35). P<0.0001 (WHI-P131 treated allo BMT group vs. Vehicle treated allo-BMT group, logrank test). See Table 1 for the statistical analysis and details of the life table analysis.

FIG. 49. Leukemia-Associated Splenomegaly is Absent in Allotransplanted Mice. [A] Spleen weight of mice from the indicated BMT and treatment groups [B] Representative spleens of mice from the indicated BMT and treatment groups.

FIG. 50. Histopathologic examination of the liver from the representative [A] syngeneic (F1) BMT recipient showing normal portal area, [B] syngeneic (F1) BMT recipient inoculated with BCL-1 cells showing extensive infiltration of portal area by immature blasts (BCL-1 cells), [C] allogeneic (B6 to F1) BMT recipient (GVHD model) showing portal lymphocytic infiltration with a bile duct infiltration and injury, [D] allogeneic (B6 to F1) BMT recipient inoculated with BCL-1 cells (GVL model) showing similar pathology as described in [C], sacrificed on day 11 post BMT/BCL-1 injection; [E] long-term WHI-P131-treated survivor of allogeneic (B6 to F1) BMT (Allo BMT->WHI-P131 group), showing normal portal area, and [F] long-term WHI-P131-treated survivor of allogeneic (B6 to F1) BMT inoculated with BCL-1 (Allo BMT+BCL-1->WHI-P131 group) recipient sacrificed on day 65 post BMT/BCL-1 injection, showing similar pathology as described for [E].

### DETAILED DESCRIPTION OF THE INVENTION

Definitions

The following definitions are used, unless otherwise described: halo is fluoro, chloro, bromo, or iodo. Alkyl, alkoxy, etc. denote both straight and branched groups; but reference to an individual radical such as propyl embraces only the straight chain radical and reference to an individual radical such as isopropyl embraces only the branched chain radical. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(W) wherein W is absent or is H, O, (C₁-C₄)alkyl, phenyl or benzyl, as well as a radical of an ortho-fused bicyclic heterocycle of about eight to ten ring atoms derived there from, particularly a benz-derivative or one derived by fusing a propylene, trimethylene, or tetramethylene diradical thereto.

Specific values listed below for radicals, substituents, and ranges, are for illustration only; they do not exclude other defined values or other values within defined ranges for the radicals and substituents

Specifically, (C₁-C₄)alkyl can be methyl, ethyl, propyl, isopropyl, butyl, iso-butyl, or sec-butyl; (C₁-C₄)alkoxy can be methoxy, ethoxy, propoxy, isopropoxy, butoxy, iso-butoxy, or sec-butoxy; (C₁-C₄)alkylthio can be methylthio, ethylthio, propylthio, isopropylthio, butylthio, or isobutylthio; (C₁-C₄)alkanoyl can be acetyl, propanoyl, butanoyl, or isobutanoyl; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine prostaglandin E₂ inhibition activity using the standard tests described herein, or using other similar tests which are well known in the art.

Compounds of the Invention

Specific JAK-3 inhibitors useful in medicaments and methods of the invention include compounds of formula I:

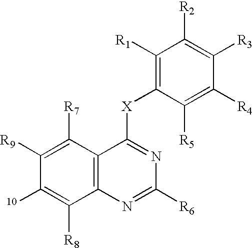

wherein:

X is HN, R₁₁N, S, O, CH₂, or R₁₁CH;

R₁₁ is hydrogen, (C₁-C₄)alkyl, or (C₁-C₄)alkanoyl;

R₁-R₈ are each independently hydrogen, hydroxy, mercapto, amino, nitro, (C₁-C₄)alkyl, (C₁-C₄)alkoxy, (C₁-C₄)alkylthio, or halo; wherein two adjacent groups of R₁-R₅ together with the phenyl ring to which they are attached may optionally form a fused ring, for example forming a naphthyl or a tetrahydronaphthyl ring; and further wherein the ring formed by the two adjacent groups of R₁-R₅ may optionally be substituted by 1, 2, 3, or 4 hydroxy, mercapto, amino, nitro, (C₁-C₄)alkyl, (C₁-C₄)alkoxy, (C₁-C₄)alkylthio, or halo; and R₉ and R₁₀ are each independently hydrogen, (C₁-C₄)alkyl, (C₁-C₄)alkoxy, halo, or (C₁-C₄)alkanoyl; or R₉ and R₁₀ together are methylenedioxy; or a pharmaceutically acceptable salt thereof. Peferably, at least one of R₂ and R₃ is hydroxy. More preferably, at least one of R₂ and R₃ is hydroxy, and one of R₁ to R₅ is halo.

A specific group of compounds are compounds of formula I wherein X is R₁₁N. Another specific group of compounds are compounds of formula I wherein X is HN.

A specific group of compounds are compounds of formula I wherein R₁, R₂, R₄,R₅, R₆, R₇, and R₁₀ are each H.

A specific group of compounds are compounds of formula I wherein R₃ is (C₁-C₄)alkoxy, hydroxy, nitro, halo, trifluoromethyl, or NR₁₂R₁₃ wherein R₁₂ and R₁₃ are each independently hydrogen, (C₁-C₄)alkyl, (C₁-C₄)alkenyl, (C₁-C₄)alkynyl, (C₃-C₈)cycloalkyl, or heterocycle. Another specific group of compounds are compounds of formula I wherein R₃ is hydroxy.

A specific group of compounds are compounds of formula I wherein R₈ is (C₁-C₄)alkoxy. Another specific group of compounds are compounds of formula I wherein R₈ is methoxy.

A specific group of compounds are compounds of formula I wherein R₉ is (C₁-C₄)alkoxy. Another specific group of compounds are compounds of formula I wherein R₉ is methoxy.

A preferred compound is 4-(4′-hydroxyl-phenyl)-amino-6,7-dimethoxyquinazoline WHI-P131; or a pharmaceutically acceptable salt thereof.

A preferred compound is 4-(3′-hydroxyl-phenyl)-amino-6,7-dimethoxyquinazoline WHI-P180; or a pharmaceutically acceptable salt thereof.

A preferred compound is 4-(3′,5′-dibromo-4′-hydroxyl-phenyl)-amino-6,7-dimethoxyquinazoline WHI-P97; or a pharmaceutically acceptable salt thereof.

A preferred compound is 4-(3′bromo-4′-hydroxyl-phenyl)-amino-6,7-dimethoxyquinazoline WHI-P154; or a pharmaceutically acceptable salt thereof.

Salts

The compounds of the invention are capable of forming both pharmaceutically acceptable acid addition and/or base salts. Base salts are formed with metals or amines, such as alkali and alkaline earth metals or organic amines. Examples of metals used as cations are sodium, potassium, magnesium, calcium, and the like. Also included are heavy metal salts such as, for example, silver, zinc, cobalt, and cerium. Examples of suitable amines are N,N′-dibenzylethylenediamine, chloroprocaine, choline, diethanolamine, ethylenediamene, N-methylglucamine, and procaine.

Pharmaceutically acceptable acid addition salts are formed with organic and inorganic acids. Examples of suitable acids for salt formation are hydrochloric, sulfuric, phosphoric, acetic, citric, oxalic, malonic, salicylic, malic, gluconic, fumaric, succinic, ascorbic, maleic, methanesulfonic, and the like. The salts are prepared by contacting the free base form with a sufficient amount of the desired acid to produce either a mono or di, etc. salt in the conventional manner. The free base forms can be regenerated by treating the salt form with a base. For example, dilute solutions of aqueous base can be utilized. Dilute aqueous sodium hydroxide, potassium carbonate, ammonia, and sodium bicarbonate solutions are suitable for this purpose. The free base forms differ from their respective salt forms somewhat in certain physical properties such as solubility in polar solvents, but the salts are otherwise equivalent to their respective free base forms for the purposes of the invention.

Treatment and Prophylactic Treatment of Diseases

The compounds and methods of the invention can be used to treat GVHD. More specifically, the compounds and methods of the invention can be used to treat GVHD without compromising the GVL function of the BMT graft.

The compounds and methods of the invention can also be used as a prophylactic treatment for preventing GVHD. More specifically, the compounds and methods of the invention can be used as a prophylactic treatment of GVHD without compromising the GVL function of the BMT graft.

The compounds of the invention can also be used for the manufacture of a medicament for use to treat or prophylcatically treat GVHD. More specifically, the compounds of the invention can be used for the manufacture of a medicament for use to treat or prophylactically treat GVHD without compromising the GVL function of the BMT graft.

Administration Methods

Methods of the invention include administration of one compound of the invention, more than one compound of the invention, or a combination of one compound of the invention and another compound, formulation, drug, or mixture not of Formula I above. In embodiments of the invention where a compound, formulation, drug or mixture not of Formula I is administered in combination with a compound of Formula I, the other compound can be administered simultaneously with or at a different time. If the other compound, formulation, drug or mixture not of Formula I is administered simultaneously with the compound of Formula I, the compound can be in the same composition of the compound of Formula I, or administered separately.

In one embodiment of the invention, a compound of the invention is administered with methotrexate. Methotrexate, a folic acid analogue, is an anticancer drug with a broad range of uses. Administration and dosage ranges of methotrexate are well known to those of skill in the art. The formula of methotrexate is given below:

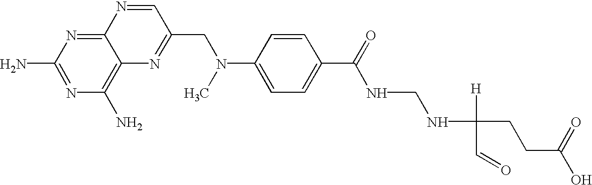

The compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes. Such pharmaceutical compositions include a compound of formula I, methotrexate and a pharmaceutically accpetable carrier. It should also be understood that methods of the invention that administer another compound, formulation, drug or mixture, such as methotrexate, can also be included in any of the formulations described herein, or known to those of skill in the art.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound may be combined with one or more excipients and used in the form of ingestible tablets, buccal tablets, troches, capsules, elixirs, suspensions, syrups, wafers, and the like. Such compositions and preparations should contain at least 0.1% of the compound. The percentage of the compositions and preparations may, of course, be varied and may conveniently be between about 2 to about 60% of the weight of a given unit dosage form. The amount of compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compound may be incorporated into sustained-release preparations and devices.

The compounds may also be administered intravenously or intraperitoneally by infusion or injection. Solutions of the compound can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the compound which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art, for example, see Jacquet et al. (U.S. Pat. No. 4,608,392), Geria (U.S. Pat. No. 4,992,478), Smith et al. (U.S. Pat.

No. 4,559,157) and Wortzman (U.S. Pat. No. 4,820,508).

Useful Dosages

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the compound should be administered to achieve peak plasma concentrations of from about 0.5 to about 75 μM, preferably, about 1 to 50 μM, most preferably, about 2 to about 30 μM. This may be achieved, for example, by the intravenous injection of a 0.05 to 5% solution of the compound, optionally in saline, or orally administered as a bolus containing about 1-100 mg of the compound. Desirable blood levels may be maintained by continuous infusion to provide about 0.01-5.0 mg/kg/hr or by intermittent infusions containing about 0.4-15 mg/kg of the compound.

The compound may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### EXAMPLES

Melting points are uncorrected. ¹H NMR spectra were recorded using a Varian Mercury 300 spectrometer in DMSO-d₆ or CDCl₃. Chemical shifts are reported in parts per million (ppm) with tetramethylsilane (TMS) as an internal standard at zero ppm. Coupling constant (J) are given in hertz and the abbreviations s, d, t, q, and m refer to singlet, doublet, triplet, quartet and multiplet, respectively. Infrared spectra were recorded on a Nicolet PROTEGE 460-IR spectrometer. Mass spectroscopy data were recorded on a FINNIGAN MAT 95, VG 7070E-HF G.C. system with an HP 5973 Mass Selection Detector. UV spectra were recorded on BECKMAN DU 7400 and using MeOH as the solvent. TLC was performed on a precoated silica gel plate (Silica Gel KGF; Whitman Inc). Silica gel (200-400 mesh, Whitman Inc.) was used for all column chromatography separations. All chemicals were reagent grade and were purchased from Aldrich Chemical Company (Milwaukee, Wis) or Sigma Chemical Company (St. Louis, Mo.).

The synthesis of a representative compound of formula I is described in Example 1. Other compounds of formula I can be prepared using procedures similar to those described in Example 1.

### Example 1

### Chemical Synthesis and Characterization of 4-(4′-hydroxyl-phenyl)-amino-6,7-dimethoxyquinazoline (6)

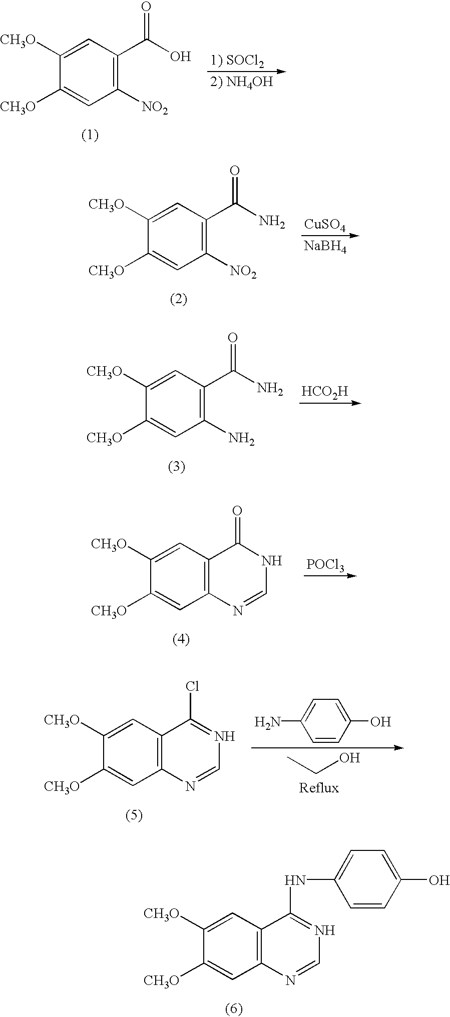

4,5-Dimethoxy-2-nitrobenzoic acid (1) was treated with thionyl chloride and then reacted with ammonia to give 4,5-dimethoxy-2-nitrobenzamide (2) as described by F. Nomoto et al. Chem. Pharm. Bull. 1990, 38, 1591-1595. The nitro group in compound (2) was reduced with sodium borohydride in the presence of copper sulfate (see C. L. Thomas Catalytic Processes and Proven Catalysts Academic Press, New York (1970)) to give 4,5-dimethoxy-2-aminobenzamide (3) which was cyclized by refluxing with formic acid to give 6,7-dimethoxyquinazoline-4(3H)-one (4). Compound (4) was refluxed with phosphorus oxytrichloride to provide compound (5), which is a useful intermediate for preparing compounds of formula I wherein X is NH. Reaction of compound (5) with the requisite aniline provided the following compounds of formula I:

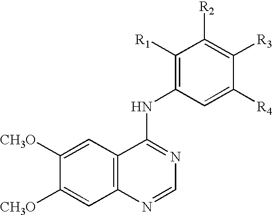

| Compound | R₁ | R₂ | R₃ | R₄ | m.p.° C. |
|---|---|---|---|---|---|
| WHI-P97 | H | Br | OH | Br | >300 |
| WHI-P111 | H | Br | Me | H | 225-228 |
| WHI-P131 | H | H | OH | H | 245-248 |
| WHI-P132 | OH | H | H | H | 255-258 |
| WHI-P154 | H | Br | OH | H | 233-233.5 |
| WHI-P180 | H | OH | H | H | 256-258 |
| WHI-P197 | H | Cl | OH | H | 245 (dec) |
| WHI-P292 | OH | H | * | * | 277-279 |

*R3 and R4 together are benzo

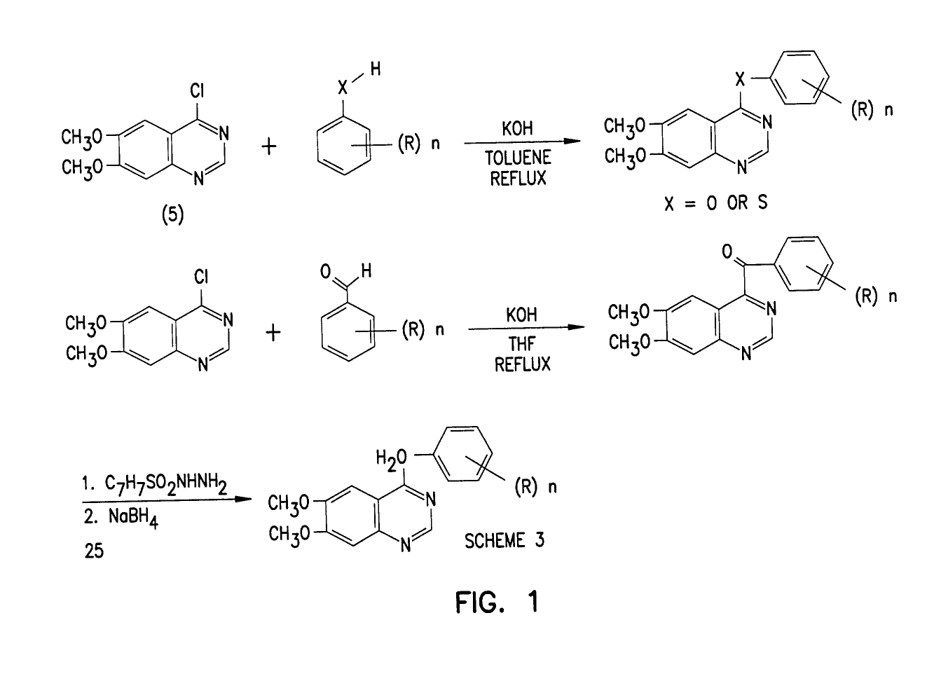

Using a procedure similar to that described above, or using procedures which are known in the art for preparing other quinazoline compounds, compounds of formula I can be prepared. For example, Compounds of formula I wherein X is S, O, or CH₂ can be prepared from a compound of formula (5) by reaction with a requsite compound of the formula PhXH as illustrated in FIG. 1.

### Example 2

### Identification of Selective JAK-3 Inhibitors

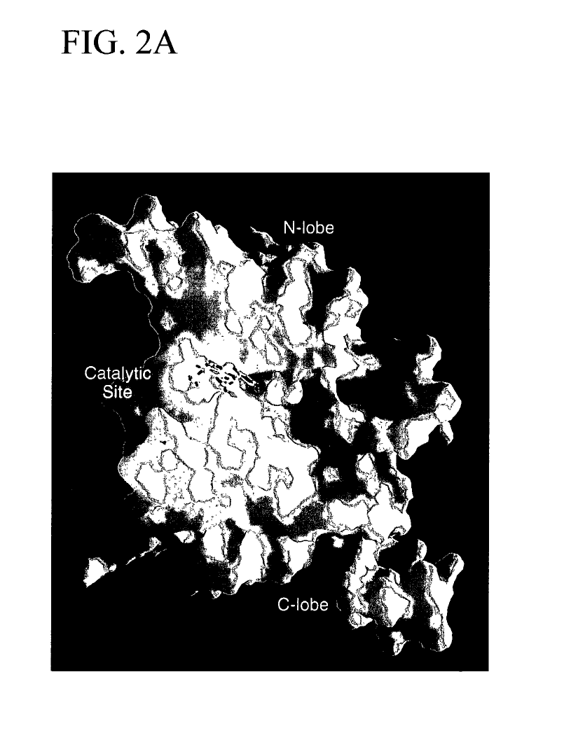

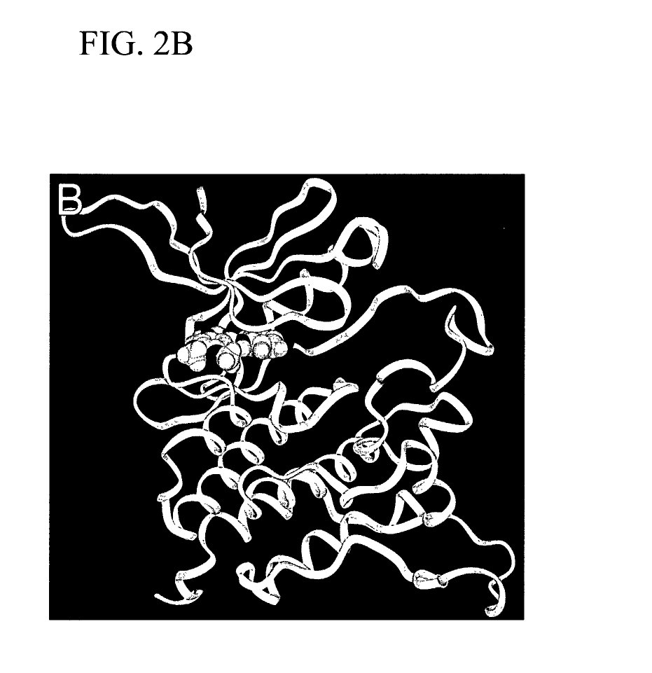

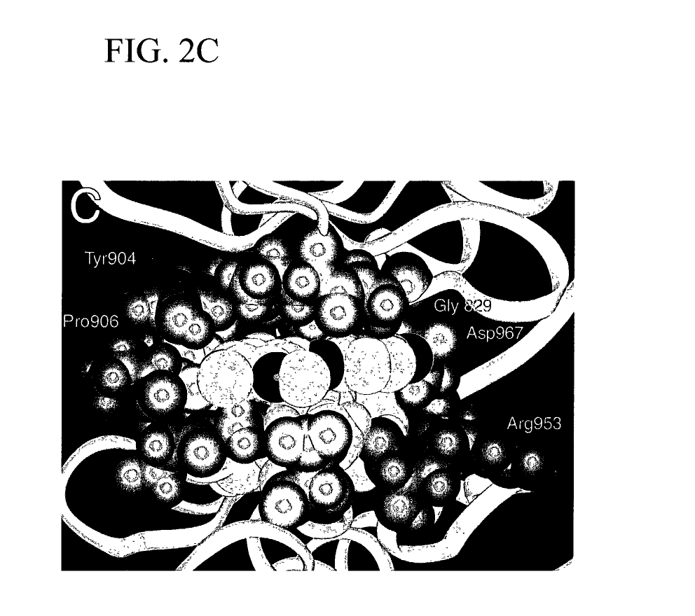

Constructing A Homology Model for the JAK3 Kinase Domain. A homology model for JAK-3 was constructed as described by E. A. Sudbeck, et al., Clinical Cancer Research, 1999, 5, 1569-1582. Because the three dimensional coordinates of the JAK3 kinase domain are currently unknown, a structural model of JAK3 was required for a docking analysis of JAK3 inhibitors. A homology model of JAK3 was constructed (FIG. 2) by using known coordinates of homologous kinase domains as a reference. The JAK3 homology model was built by first obtaining the protein sequence of JAK3 (Swiss-Prot #P52333, Univ. of Geneva, Geneva, Switzerland) from GenBank (National Center for Biotechnology Information, Bethesda, Md.) and determining the most reasonable sequence alignment for the JAK3 kinase domain relative to some template coordinates (known kinase structures such as HCK, FGFR, and IRK (Sicheri, F., et al., Nature. 385: 602-9, 1997; Mohammadi, M., et al., Cell. 86: 577-87, 1996; Mohammadi, M., et al., Science. 276: 955-60, 1997; and Hubbard, S. R., et al., Nature. 372: 746-54, 1994). This was accomplished by first superimposing the Cα coordinates of the kinase domains of HCK, FGFR, and IRK using the InsightII program to provide the best overall structural comparison (InsightII, Molecular Simulations Inc. San Diego, Calif., 1996). The sequences were then aligned based on the superimposition of their structures (amino acid sequences were aligned together if their Cα positions were spatially related to each other). The alignment accommodated such features as loops in a protein which differed from the other protein sequences. The structural superimposition was performed using the Homology module of the InsightII program and a Silicon Graphics INDIGO2 computer (Silicon Graphics, Mountain View, Calif.). The sequence alignment was done manually and produced a sequence variation profile for each superimposed Cα position. The sequence variation profile served as a basis for the subsequent sequence alignment of the JAK3 kinase with the other three proteins. In this procedure, the sequence of JAK3 was incorporated into the program and aligned with the three known kinase proteins based on the sequence variation profiles described previously. Next, a set of 3D coordinates was assigned to the JAK3 kinase sequence using the 3D coordinates of HCK as a template and the Homology module within the InsightII program. The coordinates for a loop region where a sequence insertion occurs (relative to HCK without the loop) were chosen from a limited number of possibilities automatically generated by the computer program and manually adjusted to a more ideal geometry using the program CHAIN (Sack, J. S., J. Mol. Graphics. 6: 244-245, 1988). Finally, the constructed model of the JAK3 kinase domain was subjected to energy minimization using the X-PLOR program so that any steric strain introduced during the model-building process could be relieved (Brünger, A. T. X-PLOR, A System for X-ray Crystallography and NMR). The model was screened for unfavorable steric contacts and if necessary such side chains were remodeled either by using a rotamer library database or by manually rotating the respective side chains. The procedure for homology model construction was repeated for JAK1 (SWISS-PROT #P23458) and JAK2 (Genbank #AF005216) using the JAK3 model as a structural template. The energy minimized homology models of JAK1, JAK2, and JAK3 were then used, in conjunction with energy-minimized structural models of dimethoxyquinazoline compounds, for modeling studies of JAK/dimethoxyquinazoline complexes.

Docking Procedure Using Homology Model of JAK3 Kinase Domain.

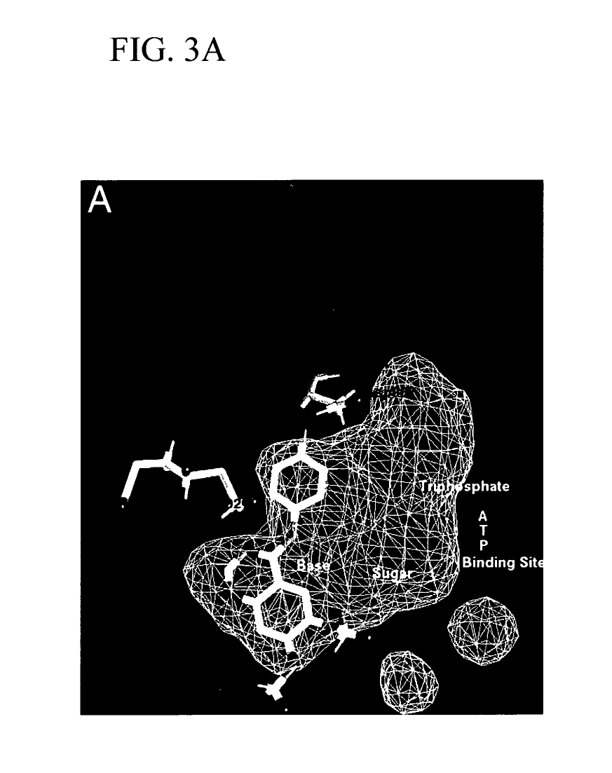

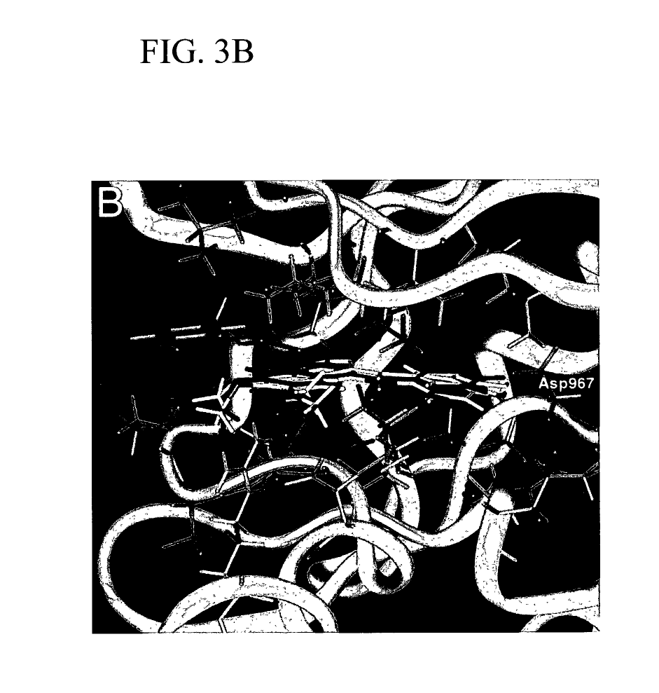

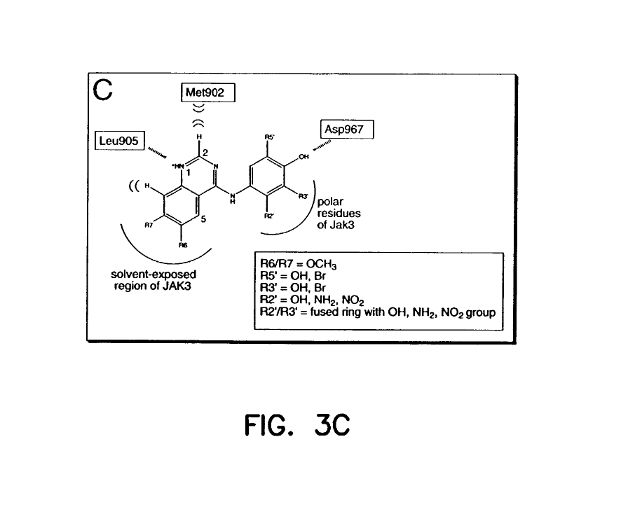

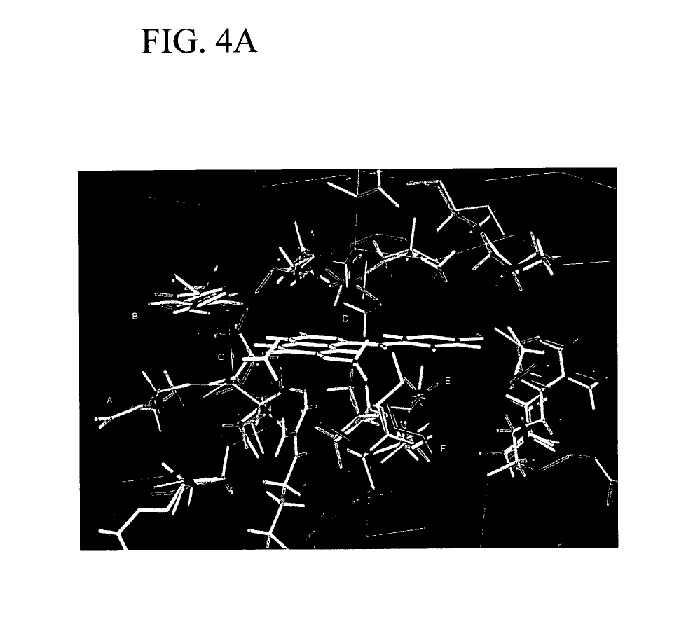

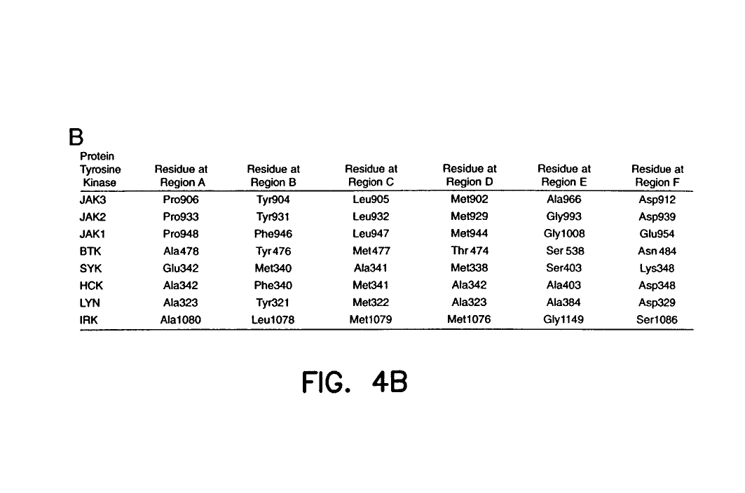

Modeling of the JAK3/dimethoxyquinazoline complexes was accomplished using the Docking module within the program INSIGHTII and using the Affinity suite of programs for automatically docking an inhibitor into a protein binding site (a similar procedure for EGFR and BTK was previously described, see Ghosh, S., et al., Clin. Can. Res. 4: 2657-2668, 1998; Mahajan, S., et al., J. Biol. Chem. 274: 9587-9599, 1999. The various docked positions of each compound were evaluated using a Ludi (Bohm, H. J. et al., J. Comput. Aided Mol. Des. 8: 243-56, 1994) scoring procedure in INSIGHTII which estimated a binding constant, Kᵢ, taking into account the predicted lipophilic, hydrogen bonding, and van der Waals interactions between the inhibitor and the protein. A comparison of the catalytic site residues of several different PTK was made by manually superimposing crystal structure coordinates of the kinase domains of IRK and HCK, and models of JAK1, JAK2, JAK3, BTK, and SYK and then identifying features in the active site which were unique to JAK3 (FIG. 3 and FIG. 4).

Chemical Synthesis of Quinazoline Derivatives.

The compounds listed in Table 1 were synthesized and characterized using literature procedures (Rewcastle, G. W., et al., J. Med. Chem. 38: 3482-7, 1995).

Table 1. Predicted interaction of protonated quinazolines with JAK3 kinase active site and measured inhibition values (IC₅₀ values) from JAK3 kinase assays.

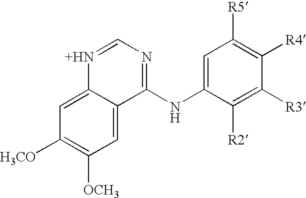

| | | | | | | | | | | | Mol. | | |
|---|---|---|---|---|---|---|---|---|---|---|---|---|---|
| | | | | | | H | | | Total | | Surf. | Mol. | |
| Cmpd | | | | | # H | Bond | Lipo | Contact | Binding | Est. | Area | Vol. | IC₅₀ |
| Name | R5′ | R4′ | R3′ | R2′ | Bonds | Score | Score | Score | Score | Ki (μM) | (Å²) | (Å³) | (μM) |
| WHI- | H | OH | H | H | 3 | 188 | 476 | 64 | 568 | 2.3 | 276 | 261 | 9.1 |
| P131 | | | | | | | | | | | | | |
| WHI-P97 | Br | OH | Br | H | 3 | 156 | 559 | 65 | 622 | 0.6 | 314 | 307 | 11.0 |
| WHI- | H | OH | Br | H | 3 | 171 | 512 | 64 | 587 | 1.4 | 296 | 284 | 27.9 |
| P154 | | | | | | | | | | | | | |
| WHI-P79 | H | H | Br | H | 1 | 9 | 531 | 63 | 444 | 36 | 278 | 272 | >300 |
| WHI- | H | H | H | OH | 1 | 82 | 476 | 66 | 462 | 25 | 269 | 264 | >300 |
| P132 | | | | | | | | | | | | | |
| WHI- | H | CH₃ | Br | H | 1 | 52 | 502 | 58 | 458 | 46 | 309 | 291 | >300 |
| P111 | | | | | | | | | | | | | |
| WHI- | Br | H | H | Br | 0 | 0 | 541 | 62 | 445 | 52 | 306 | 297 | >300 |
| P112 | | | | | | | | | | | | | |
| WHI- | H | H | H | H | 0 | 0 | 510 | 64 | 414 | 72 | 266 | 252 | >300 |
| P258 | | | | | | | | | | | | | |

Immune Complex Kinase Assays.

Sf21 (IPLB-SF21-AE) cells (Vassilev, A., et al., J. Biol. Chem. 274: 1646-1656, 1999) derived from the ovarian tissue of the fall armyworm Spodotera frugiperda, were obtained from Invitrogen (Carlsbad, Calif.) and maintained at 26-28° C. in Grace's insect cell medium supplemented with 10% FBS and 1.0% antibiotic/antimycotic (GIBCO-BRL). Stock cells were maintained in suspension at 0.2-1.6×10⁶/ml in 600 ml total culture volume in 1 L Bellco spinner flasks at 60-90 rpm. Cell viability was maintained at 95-100% as determined by trypan blue dye exclusion. Sf21 cells were infected with a baculovirus expression vector for BTK, SYK, JAK1, JAK2, or JAK3. Cells were harvested, lysed (10 mM Tris pH 7.6, 100 mM NaCl, 1% Nonidet P-40, 10% glycerol, 50 mM NaF, 100 μM Na₃VO₄, 50 μg/ml phenylmethylsulfonyl fluoride, 10 μg/ml aprotonin, 10 μg/ml leupeptin), the kinases were immunoprecipitated from the lysates, and their enzymatic activity assayed, as reported (Vassilev, A., et al., J. Biol. Chem. 274: 1646-1656, 1999; Uckun, F. M., et al., Science. 22: 1096-1100, 1996; Goodman, P. A., et al., J. Biol. Chem. 273: 17742-48, 1998; Mahajan, S., et al., Mol. Cell. Biol. 15: 5304-11, 1995; and Uckun, F. M., et al., Science. 267: 886-91, 1995). The immunoprecipitates were subjected to Western blot analysis as previously described (Vassilev, A., et al., J. Biol. Chem. 274: 1646-1656, 1999; and Uckun, F. M., et al., Science. 22: 1096-1100, 1996).

For insulin receptor kinase (IRK) assays, HepG2 human hepatoma cells grown to approximately 80% confluency were washed once with serum-free DMEM and starved for 3 hours at 37° in a CO₂ incubator. Subsequently, cells were stimulated with insulin (Eli Lilly and Co., Indianapolis, Ind., cat# CP-410;10 units/ml/10×10⁶ cells) for 10 minutes at room temperature. Following this IRK activation step, cells were washed once with serum free medium, lysed in NP-40 buffer and IRK was immunoprecipitated from the lysates with an anti-IRβ antibody (Santa Cruz Biotechnology, Santa Cruz, Calif., Cat.# sc-711, polyclonal IgG). Prior to performing the immune complex kinase assays, the beads were equilibrated with the kinase buffer (30 mM Hepes pH 7.4, 30 mM NaCl, 8 mM MgCl₂, 4 mM MnCl₂). LYN was immunoprecipitated from whole cell lysates of NALM-6 human leukemia cells as previously reported (Uckun, F. M., et al., Science. 267: 886-91, 1995; and Uckun, F. M., et al., Journal of Biological Chemistry, 271: 6396-6397, 1996).

In JAK3 immune complex kinase assays (17, 22), KL-2 EBV-transformed human lymphoblastoid B cells (native JAK3 kinase assays) or insect ovary cells (recombinant JAK3 kinase assays) were lysed with NP-40 lysis buffer (50 mM Tris, pH 8, 150 mM NaCl, 5 mM EDTA, 1% NP-40, 100 μM sodium orthovanadate, 100 μM sodium molybdate, 8 μg/ml aprotinin, 5 μg/ml leupeptin, and 500 μM PMSF) and centrifuged 10 min at 13000×g to remove insoluble material. Samples were immunoprecipitated with antisera prepared against JAK3. The antisera were diluted and immune complexes collected by incubation with 15 μl protein A Sepharose. After 4 washes with NP-40 lysis buffer, the protein A Sepharose beads were washed once in kinase buffer (20 mM MOPS, pH 7, 10 mM MgCl₂) and resuspended in the same buffer. Reactions were initiated by the addition of 25 μCi [γ-32P] ATP (5000 Ci/mMole) and unlabeled ATP to a final concentration of 5 μM. Reactions were terminated by boiling for 4 min in SDS sample buffer. Samples were run on 9.5% SDS polyacrylamide gels and labeled proteins were detected by autoradiography. Following electrophoresis, kinase gels were dried onto Whatman 3M filter paper and subjected to phosphorimaging on a Molecular Imager (Bio-Rad, Hercules, Calif.) as well as autoradiography on film. For each drug concentration, a kinase activity index (KA) was determined by comparing the kinase activity in phosphorimager units (PIU) to that of the baseline sample. In some experiments, cold kinase assays were performed, as described by (Uckun, F. M., et al., Clin. Can. Res. 4. 901-912, 1998).

Electrophoretic Mobility Shift Assays (EMSAs).

EMSAs were performed to examine the effects of dimethoxyquinazoline compounds on cytokine-induced STAT activation in 32Dc11/IL2Rβ cells (gift from Dr. James Ihle, St. Jude Children's Research Hospital), as previously described (Goodman, P. A., et al., J. Biol. Chem. 273: 17742-48, 1998).

Mitochondrial Membrane Potential Assessment.

To measure the changes in mitochondria, cells were incubated with WHI-P131 at concentrations ranging from 7.4 μg/ml (25 μM) to 30 μg/ml (200 μM) for 24 h or 48 h, stained with specific fluorescent dyes and analyzed with flow cytometer. Mitochondrial membrane potential (Δψ) was measured using two dyes including a lipophillic cation 5,5′,6,6′-tetrachloro-1,1′,3,3′-tetraethyl-benzimidazolylcarbo cyanine iodide (JC-1) and a cyanine dye, 1,1′,3,3,3′,3′-hexamethylindodicarbo cyanine iodide (DiIC1 (27-29)) obtained from Molecular Probes (Eugene, Oreg.). JC-1 is a monomer at 527 nm after being excited at 490 nm; with polarization of Δψm, J-aggregates are formed that shift emission to 590 nm (30). This can be detected on a flow cytometer by assessing the green signal (at 527 nm) and green-orange signal (at 590 nm) simultancously, creating an index of the number of cells polarized and depolarized mitochondria. DiIC1, a cyanine dye which is amphioatheic and cationic, concentrates in energized mitochondria and has been used in a variety of studies to measure the mitochondrial membrane potential (Fujii, H., et al., Histochem J. 29: 571-581, 1997; Mancini, M., et al., J. Cell Biol. 138: 449-469, 1997; and Petit, P. X., et al., J. Cell Biol. 130. 157-167, 1995). Cells were also stained with DiIC1 at 40 nM concentration for 30 min in the dark as described for JC-1. The cells were analyzed using a Vantage Becton Dickinson (San Jose, Calif.) cell sorter equipped with HeNe laser with excitation at 635 nm and the fluorescence was collected at 666 nm. Mitochondrial Mass Determination.

Relative mitochondrial mass was measured by using Becton Dickinson Calibur flow cytometry and the fluorescent stain 10-n-nonyl-acridine orange (NAO), which binds the mitochondrial phospholipid cardiolipin, that has been extensively used to provide an index of mitochondrial mass (Maftah, A., et al., Biochem. Biophys. Res. Commun. 164. 185-190, 1989).

Homology Model of JAK3 Kinase Domain.

The three-dimensional coordinates of JAK3 used in the protein/inhibitor modeling studies were constructed based on a structural aligment with the sequences of known crystal structures of the kinase domains of three protein tyrosine kinases (PTKs) (kinase domains of HCK (9), FGFR (11), and IRK (37)), as detailed in Materials and Methods. FIGS. 2A and 1B show the homology model of the JAK3 kinase domain, which is composed of an N-terminal lobe and a C-terminal lobe which are linked by a hinge region near the catalytic (ATP-binding) site. The catalytic site is a pocket located in the central region of the kinase domain, which is defined by two β-sheets at the interface between the N and C lobes. The opening to the catalytic site is solvent accessible and facilitates binding of ATP. Small molecule inhibitors can also bind to the catalytic site which results in an attenuation of PTK activity by inhibiting ATP binding. An analysis of the JAK3 model revealed specific features of the catalytic site which can be described as a quadrilateral-shaped pocket (FIG. 2C). The opening of the pocket is defined by residues Pro906, Ser907, Gly908, Asp912, Arg953, Gly829, Leu828, and Tyr904 (blue residues, FIG. 2C). The far wall deep inside the pocket is lined with Leu905 (Cα backbone), Glu903, Met902, Lys905, and Asp967 (pink residues, FIG. 2C), and the floor of the pocket is lined by Leu905 (side chain), Val884, Leu956, and Ala966 (yellow residues, FIG. 2C). Residues defining the roof of the pocket include Leu828, Gly829, Lys830, and Gly831 (uppermost blue residues, FIG. 2C). FIGS. 2C and 3A illustrate that the catalytic site of the JAK3 model has approximate dimensions of 8×11×20 Å and an available volume for binding of approximately 530 Å³. According to the model, the solvent exposed opening to the binding region would allow inhibitors to enter and bind if the molecule contains some planarity.

While most of the catalytic site residues of the JAK3 kinase domain were conserved relative to other PTKs, a few specific variations were observed (FIG. 4). These differences include an alanine residue in BTK, IRK, and HCK/LYN (region A, FIG. 4A) which changes to Glu in SYK and Pro906 in JAK3. At region B, a tyrosine residue is conserved in JAK3 (Tyr904), BTK, and LYN, but changes to Phe in HCK (which is the only apparent residue difference between HCK and LYN relevant to inhibitor binding), Met in SYK, and Leu in IRK. Region C shows a methionine residue which is conserved in BTK, IRK, and HCK/LYN, but changes to Leu905 in JAK3 and to Ala in SYK. Region D shows Met902 in JAK3, which is conserved in SYK and IRK but changes to Thr in BTK and to a much smaller residue, Ala, in LYN and HCK. This Met902 residue in JAK3, which is located on the back wall of the pocket and protrudes in toward the center of the pocket volume, can significantly affect the shape of the binding pocket. At this location, the extended conformation of the Met902 side chain can hinder the close contact of inhibitors with residues lining the back wall of the pocket and with the hinge region, relative to other kinases with smaller residues here such as BTK (Thr) and HCK/LYN (Ala). Ala966 in region E is conserved in HCK/LYN but changes to Gly in IRK and to the more hydrophilic residue Ser in BTK and SYK. Region F, which is farther away from the inhibitor location, is the least conserved region of the catalytic site and contains Asp912 in JAK3, Asn in BTK, Lys in SYK, Ser in IRK, and Asp in HCK/LYN (FIG. 4). These residue identity differences between tyrosine kinases provide the basis for designing selective inhibitors of the JAK3 kinase domain.

Structure-Based Design and Synthesis of JAK3 Inhibitors.

A computer docking procedure was used to predict how well potential inhibitors could fit into and bind to the catalytic site of JAK3 and result in kinase inhibition (FIG. 3B). The dimethoxyquinazoline compound WHI-P258 (4-(phenyl)-amino-6,7-dimethoxyquinazoline) contains two methoxy groups on the quinazoline moiety but no other ring substituents. Molecular modeling studies using the homology model of JAK3 kinase domain suggested that WHI-P258 would fit into the catalytic site of JAK3, but probably would not bind very tightly due to limited hydrogen bonding interactions. Asp967, a key residue in the catalytic site of JAK3, can form a hydrogen bond with molecules binding to the catalytic site, if such molecules contain a hydrogen bond donor group such as an OH group. WHI-P258, however, does not contain an OH group and therefore would not interact as favorably with Asp967. We postulated that the presence of an OH group at the 4′ position of the phenyl ring of WHI-P258 would result in stronger binding to JAK3 because of added interactions with Asp967. A series of dimethoxyquinazoline compounds were designed and synthesized to test this hypothesis.

An estimation of the molecular volume for the compounds is provided in Table 1.

A summary of structural features of the designed dimethoxyquinazoline compounds which were observed to be relevant for binding to the catalytic site of JAK3 is shown in FIG. 3C. The approximate molecular volumes of the compounds in Table 1 range from 252A to 307A, which are small enough to fit into the 530 Å³ binding site of JAK3 kinase. Table 1 also lists the results of molecular modeling studies including estimated binding constants (i.e., Kᵢ values) for the compounds which were docked into the JAK3 catalytic site. The compounds which were evaluated in docking studies contain substitutions of similar functional groups at different positions on the phenyl ring.

The conformations of the energy-minimized docked models of the compounds listed in Table 1 were relatively planar, with dihedral angles of approximately 4-18° between the phenyl ring and quinazoline ring system. This conformation allows the molecule to fit more easily into the catalytic site of JAK3. All of the listed compounds contain a ring nitrogen (N1), which can form a hydrogen bond with NH of Leu905 in the hinge region of JAK3. When N1 is protonated, the NH can instead interact with the carbonyl group in Leu905 of JAK3. The presence of an OH group at the 4′ position on the phenyl ring was anticipated to be particularly important for binding to the catalytic site of JAK3. WHI-P131 (estimated Kᵢ=2.3 μM), WHI-P154 (estimated Kᵢ=1.4 μM), and WHI-P97 (estimated Kᵢ=0.6 μM) shown in Table 1 were predicted to have favorable binding to JAK3 and potent JAK3 inhibitory activity because they contain a 4′ OH group on the phenyl ring which can form a hydrogen bond with Asp967 of JAK3, contributing to enhanced binding. However, the 2′ OH group of WHI-P132 is not in the right orientation to interact with Asp967 and it probably would form an intramolecular hydrogen bond with the quinazoline ring nitrogen, which may contribute to a significantly lower affinity of WHI-P132 for the catalytic site of JAK3. The relatively large bromine substituents (WHI-P97, WHI-P154) can increase the molecular surface area in contact with binding site residues if the molecule can fit into the binding site. Modeling of WHI-P154 and WHI-P97 showed that there is enough room to accommodate the bromine groups if the phenyl ring is tilted slightly relative to the fused ring group of the molecule. The results from the modeling studies prompted the hypothesis that WHI-P131, WHI-P154, and WHI-P97 would exhibit potent JAK3-inhibitory activity. In order to test this hypothesis and validate the predictive value of the described JAK3 homology model, we synthesized WHI-P131, WHI-P154, WHI-P97, and 5 other dimethoxyquinazoline compounds listed in Table 1.

Inhibition of JAK3 by Rationally Designed Dimethoxy-Quinazoline Compounds.

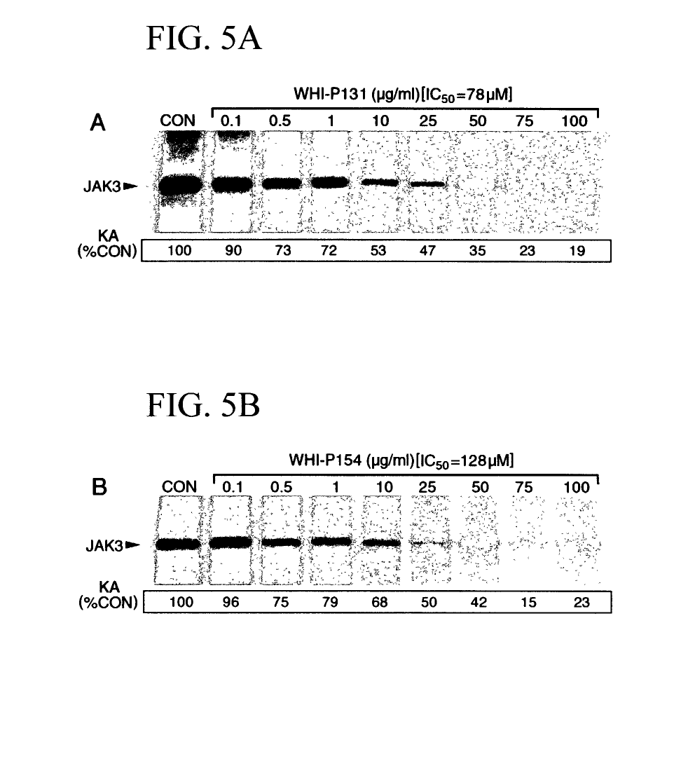

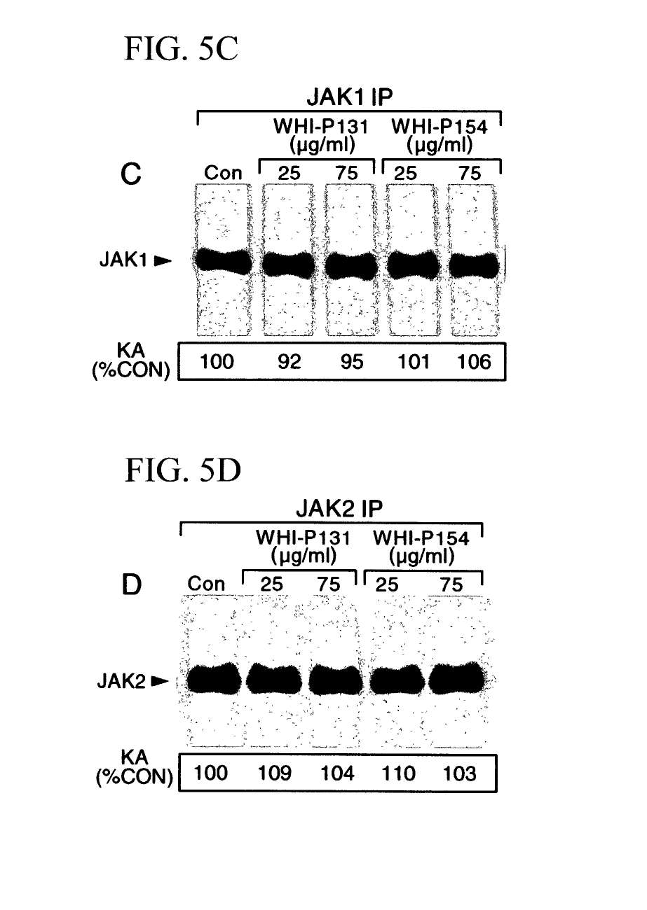

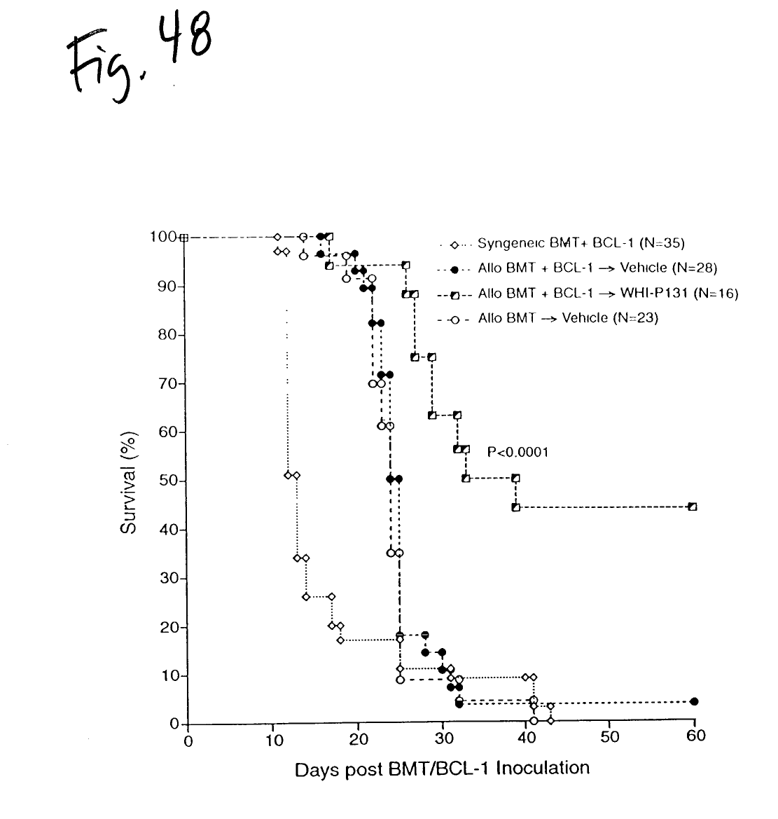

We first used immune complex kinase assays to compare the effects of the synthesized dimethoxyquinazoline compounds on the enzymatic activity of human JAK3 immunoprecipitated from the KL2 EBV-transformed human lymphoblastoid B cell line. WHI-P131, WHI-P154, and WHI-P97, which had very similar estimated Kᵢ values ranging from 0.6 μM to 2.3 μM and were predicted to show significant JAK3 inhibitory activity at micromolar concentrations (which was not the case for the other compounds which had estimated Kᵢ values ranging from 25 μM to 72 EM), inhibited JAK3 in concentration-dependent fashion. The measured IC₅₀ values were 9.1 μM for WHI-P131, 11.0 μM for WHI-P97, and 27.9 μM for WHI-P154, but >300 μM for all the other dimethoxyquinazoline compounds (Table 1). WHI-P131 and WHI-P154 were also tested against recombinant murine JAK3 expressed in a baculovirus vector expression system and inhibited JAK3 in a concentration-dependent fashion with an IC₅₀ value of 23.2 μg/ml (˜78 μM, FIG. 5A) and 48.1 μg/ml (˜128 μM, FIG. 5B), respectively. The ability of WHI-P131 and WHI-P154 to inhibit recombinant JAK3 was confirmed in 4 independent experiments. These kinase assay results are consistent with our modeling studies described above.

Importantly, WHI-P131 and WHI-P154 did not exhibit any detectable inhibitory activity against recombinant JAK1 or JAK2 in immune complex kinase assays (FIGS. 5C and 5D). Electrophoretic Mobility Shift Assays (EMSAs) were also performed to confirm the JAK3 specificity of these dimethoxyquinazoline compounds by examining their effects on cytokine-induced STAT activation in 32Dc11/IL2RP cells. As shown in FIG. 5E, both WHI-P131 (10 μg/ml=33.6 μM) and WHI-P154 (10 μg/ml=26.6 μM) (but not the control compound WHI-P132, 10 μg/ml=33.6 μM) inhibited JAK3-dependent STAT activation after stimulation with IL-2, but they did not affect JAK1/JAK2-dependent STAT activation after stimulation with IL-3. Modeling studies suggest that this exquisite JAK3 specificity could in part be due to an alanine residue (Ala966) which is present in the catalytic site of JAK3 but changes to glycine in JAK1 and JAK2. This alanine group which is positioned near the phenyl ring of the bound dimethoxyquinazoline compounds can provide greater hydrophobic contact with the phenyl group and thus can contribute to higher affinity relative to the smaller glycine residue in this region of the binding site in JAK1 and JAK2. However, an accurate interpretation of these remarkable differences in sensitivity of JAK3 versus JAK1 and JAK2 to WHI-131 and WHI-P154 will need to await the determination of the X-ray crystal structures of these kinases since simple amino acid discrepancies in their catalytic sites could result in pronounced structural differences.

Specificity of WHI-P131 as a Tyrosine Kinase Inhibitor.

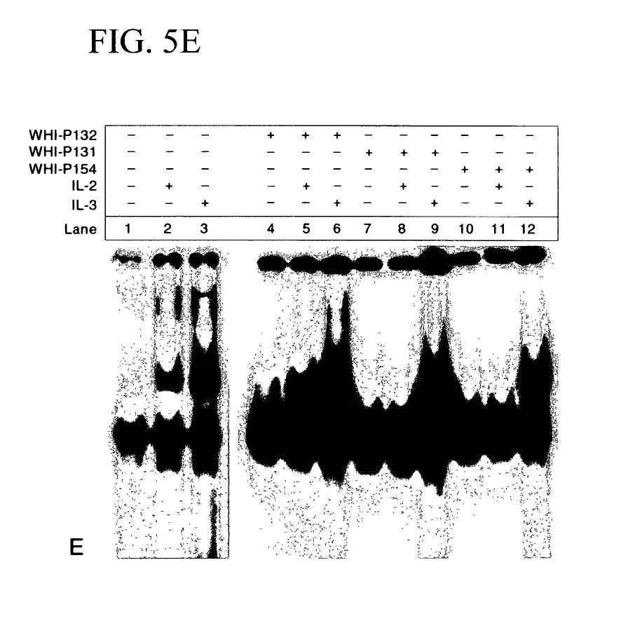

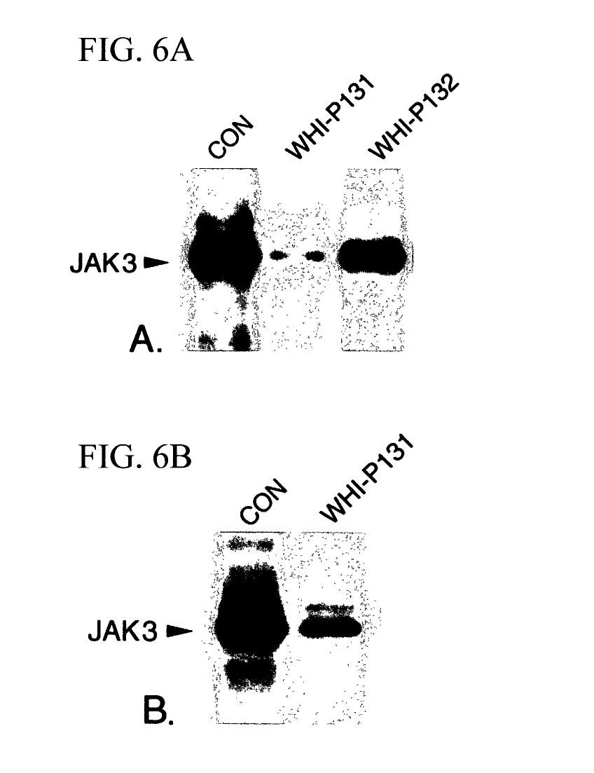

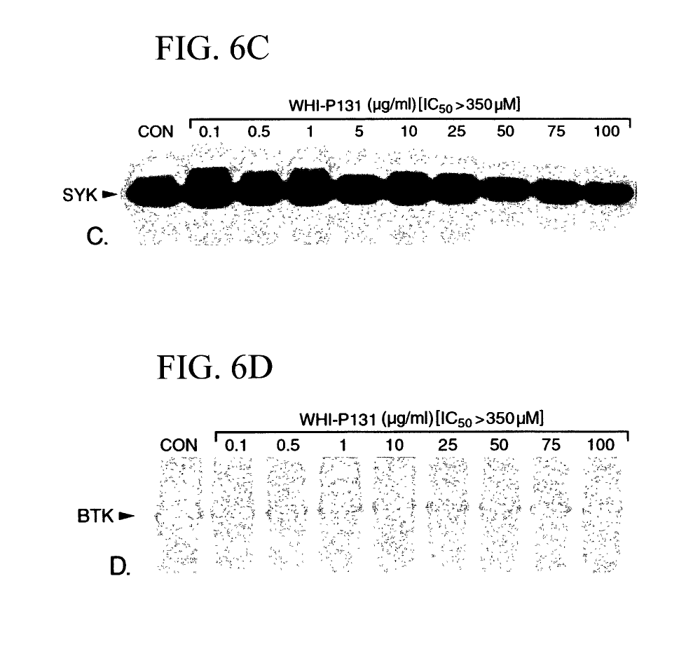

Compound WHI-P131 was selected for further experiments designed to examine the sensitivity of non-Janus family protein tyrosine kinases to this novel dimethoxyquinazoline class of JAK3 inhibitors. The inhibitory activity of WHI-P131 against JAK3 was specific since it did not affect the enzymatic activity of other protein tyrosine kinases (Table 1, FIG. 6), including the ZAP/SYK family tyrosine kinase SYK (FIG. 6C), TEC family tyrosine kinase BTK (FIG. 6D), SRC family tyrosine kinase LYN (FIG. 6E), and receptor family tyrosine kinase IRK (FIG. 6F) even at concentrations as high as 350 μM.

A structural analysis of these PTKs was performed using the crystal structures of HCK (which served as a homology model for LYN) and IRK, and constructed homology models of JAK3, BTK, and SYK. This analysis revealed some nonconserved residues located in the catalytic binding site of the different tyrosine kinases which may contribute to the specificity of WHI-P131 (FIG. 4). One such residue which is located closest to the docked inhibitors is Ala966 in JAK3 (shown in region E in FIG. 4) which may provide the most favorable molecular surface contact with the hydrophobic phenyl ring of WHI-P131. The fact that WHI-P131 did not inhibit LYN, even though LYN contains the Ala residue conserved in JAK3 (Ala966), suggests that other factors (residue differences) contribute to this selectivity. Other nonconserved residues in the catalytic site of tyrosine kinases are shown in regions A to F (FIG. 4). All of these differences in residues, especially residues which directly contact the bound inhibitor, may play an important role in the observed specificity of WHI-P131 for JAK3.

Example 2 demonstrates that a novel homology model of the JAK3 kinase domain can be used for structure-based design and synthesis of potent and specific inhibitors of JAK3.

Finally, the homology model uniquely indicates that the active site of JAK3 measures approximately 8 Å×11 Å×20 Å with an approximate 530 Å³ volume available for inhibitor binding. Our modeling studies using the constructed homology model of JAK3 kinase also showed that there is significant opportunity for improvement of the quinazoline inhibitors. The JAK3 model shows that there is additional volume in the ATP-binding site which can be better utilized by quinazoline derivatives. The average molecular volume of our dimethoxyquinazoline compounds is 277 Å³, which is well below the estimated total volume of the binding site, 530 Å³. This leaves opportunities for the design of new inhibitors which have slightly larger functional groups at the 2′ and 3′ positions of the phenyl ring. Structural and chemical features of dimethoxyquinazoline compounds which are proposed to facilitate their binding to the Jak3 catalytic site include the following features which are illustrated in FIG. 3C: 1) The presence of a 4′-OH group on the phenyl ring, 2) the presence of a hydrogen-bond acceptor (N, carbonyl, OH) near Leu905 NH, or a hydrogen-bond donor (NH, OH) near the Leu905 carbonyl, 3) a relatively planar molecular shape to allow access to the binding site, 4) the ability to fit into a 530A³ space defined by the residues lining the Jak3 catalytic site. These predicted binding preferences to JAK3 residues in the catalytic site can be used for the design of new and more potent inhibitors of JAK3.

### Example 3

### Leukemia Assays

The ability of a compound to act as an anti-leukemic agent can be determined using assays that are known in the art, or can be determined using assays similar to those described in this example.

The following cell lines were used in various biological assays: NALM-6 (pre-B-ALL), LC1;19 (Pre-B-ALL), DAUDI (B-ALL), RAMOS (B-ALL), MOLT-3 (T-ALL), HL60 (AML), BT-20 (breast cancer), M24-MET (melanoma), SQ20B (squamous cell carcinoma), and PC3 (prostate cancer). These cell lines were maintained in culture as previously reported (16, 17, 20, 24, 32, 33). Cells were seeded in 6-well tissue culture plates at a density of 50×10⁴cells/well in a treatment medium containing various concentrations of compound 6 and incubated for 24-48 hours at 37° C. in a humidified 5% CO₂ atmosphere.

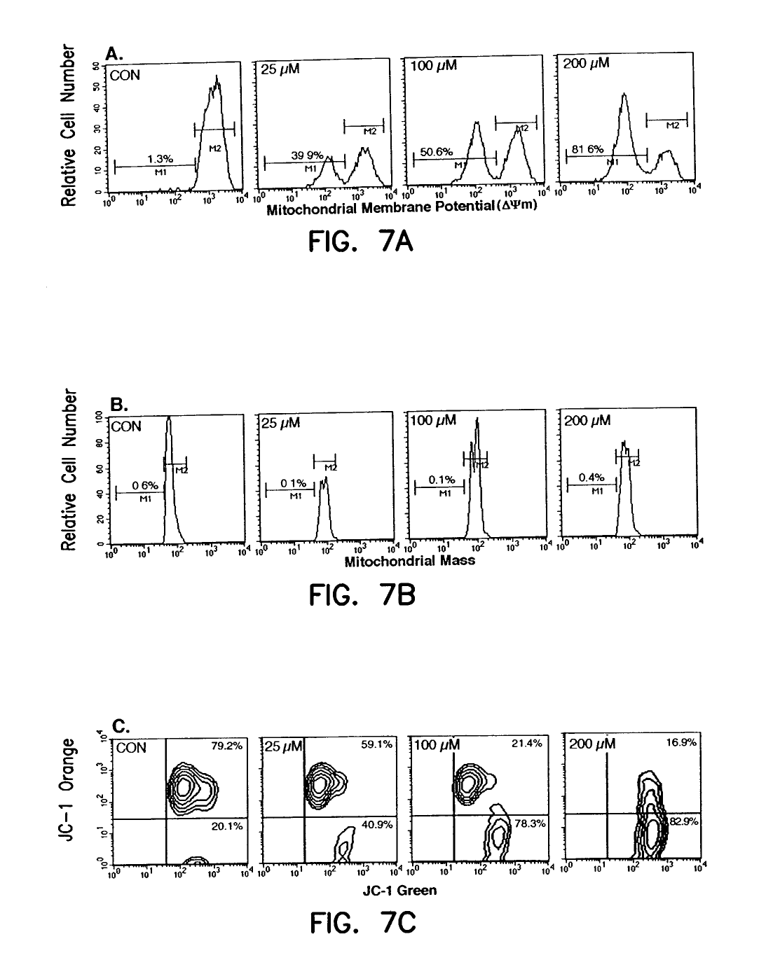

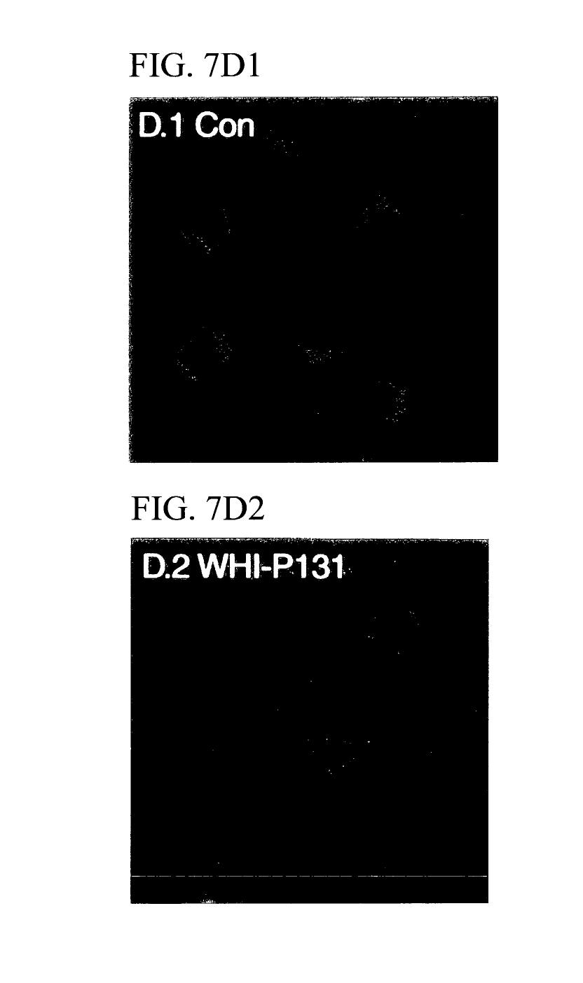

To test the cytotoxicity of compound 6 against JAK-3 expressing human leukemia cells, leukemic cells were exposed to this JAK3 inhibitor and monitored for apoptosis-associated changes in mitochondrial membrane potential (A™) and mitochondrial mass using specific fluorescent mitochondrial probes and multiparameter flow cytometry. To measure changes in Δωm, DiIC1 (which accumulates in energized mitochondria) was used, whereas the mitochondrial mass was determined by staining the cells with NAO, a fluorescent dye that binds to the mitochondrial inner membrane independent of energetic state. Treatment of NALM-6 leukemia cells with compound 6 at 7.4 μg/ml (25 μM) to 60 μg/ml (200 μM) for 24 h or 48 h increased the number of depolarized mitochondria in a concentration- and time-dependent manner as determined by flow cytometry using DiIC1 (27-29) (FIG. 7A). As shown in FIG. 7A, the fraction of DiIC1-negative cells with depolarized mitochondria increased from 1.3% in vehicle treated control cells to 81.6% in cells treated with 200 μM compound 6 for 48 hours. The average EC₅₀ values for compound 6 induced depolarization of mitochondria, as measured by decreased DiIC1 staining, were 79.3 μM for a 24 hour treatment and 58.4 μM for a 48 hour treatment. The observed changes in A™ were not due to loss in mitochondrial mass, as confirmed by a virtually identical staining intensity of NAO in the treated and untreated NALM-6 cells (FIG. 7B). To further confirm this relative change in Δψm, we used JC-1, a mitochondrial dye, which normally exists in solution as a monomer emitting green fluorescence and assumes a dimeric configuration emitting red fluorescence in a reaction driven by mitochondrial transmembrane potential (Smiley, S. T., et al., Proc. Natl. Acad. Sci. U.S.A. 88: 3671-3675, 1991). Thus, the use of JC-1 allows simultaneous analysis of mitochondrial mass (green fluorescence) and mitochondrial transmembrane potential (red/orange fluorescence). After treatment of NALM-6 cells with compound 6 at increasing concentrations ranging from 25 μM to 200 μM and with increasing duration of exposure of 24 h or 48 h, we observed a progressive dissociation between Δψm and mitochondrial mass, with decrement in JC-1 red/orange fluorescence without a significant corresponding drop in JC-1 green fluorescence (FIGS. 7C&D).

As shown in FIG. 7C, the fraction of JC-1 red/orange fluorescence-negative cells decreased from 79.2% in vehicle-treated control cells to 16.9% in cells treated with 200 μM compound 6 for 48 hours. The corresponding values for JC-1 green fluorescence were 99.3% for vehicle-treated cells and 99.8% for compound 6-treated (200 μM×48 hours) cells. The average EC₅₀ values for compound 6 induced depolarization of mitochondria, as measured by decreased JC-1 red/orange fluorescence were 94.2 μM for a 24 hour treatment and 50.4 μM for a 48 hour treatment. FIG. 7D compares the single color (red/orange) fluorescent confocal images of vehicle-treated and compound 6-treated (100 μM×48 hours) NALM-6 cells stained with JC-1. These results collectively demonstrate that compound 6 causes a significant decrease in mitochondrial transmembrane potential in NALM-6 human leukemia cells.

Apoptosis Assays

Cells were examined for apoptotic changes after treatment with compound 6 by the in situ terminal dideoxynucleotidyl transferase-mediated dUTP end-labeling (TUNEL) assay using the ApopTag apoptosis detection kit (Oncor, Gaithersburg, Md.) according to the manufacturer's recommendations, as detailed in our earlier reports (Zhu, D. -M., et al., Clin. Can. Res. 4: 2967-2976, 1998; D'Cruz, O., P., G., and Uckun, F. M. Biology of Reproduction. 58. 1515-1526, 1998).

To detect apoptotic fragmentation of DNA, cells were harvested after a 24 hour exposure at 37° C. at 1, 3, and/or 10 μM concentrations. DNA was prepared from Triton-X-100 lysates for analysis of fragmentation (21). In brief, cells were lysed in hypotonic 10 mmol/L Tris-HCl (pH 7.4), 1 mmol/L EDTA, 0.2% Triton-X-100 detergent; and subsequently centrifuged at 11,000 g. To detect apoptosis-associated DNA fragmentation, supernatants were electrophoresed on a 1.2% agarose gel, and the DNA fragments were visualized by ultraviolet light after staining with ethidium bromide.

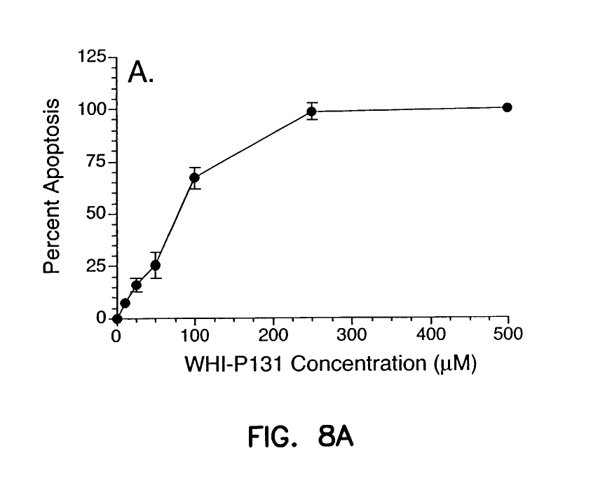

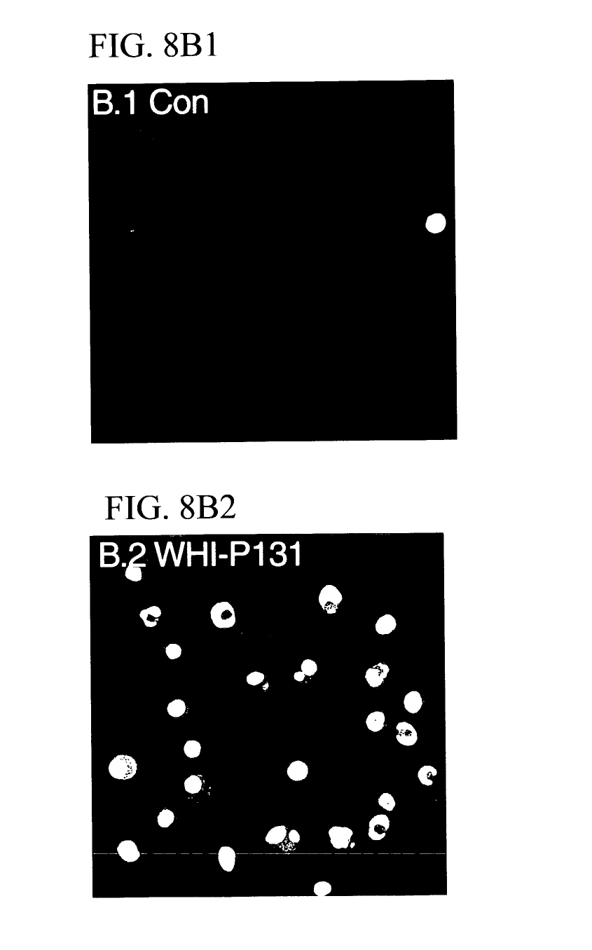

To confirm that compound 6 can induce apoptosis in leukemia cells, the TdT-mediated labeling of 3′-OH termini with digoxigenin-conjugated UTP using the in situ TUNEL assay method combined with confocal laser scanning microscopy was employed. At 48 hours after treatment with compound 6 at concentrations ranging from 10 μM to 500 μM, NALM-6 cells were examined for digoxigenin-dUTP incorporation using FITC-conjugated anti-digoxigenin (green fluorescence) and propidium iodide counterstaining (red fluorescence). The percentage of apoptotic cells increased in a concentration-dependent fashion with an average EC₅₀ value of 84.6 μM (FIG. 8A). FIG. 8B.1 and 8B.2 depict the two-color confocal microscopy images of vehicle-treated control cells and cells treated with 100 μM compound 6. Compound 6-treated cells showed apoptotic yellow nuclei (=superimposed green and red fluorescence) (FIG. 8B.2). Further evidence for apoptosis was observed in DNA fragmentation assays. Because of their exquisite sensitivity in detecting DNA fragments released from a small percentage of apoptotic cells, the DNA gel assays of apoptosis are uniquely suited to examine the nonspecific toxicity of new antileukemic agents.

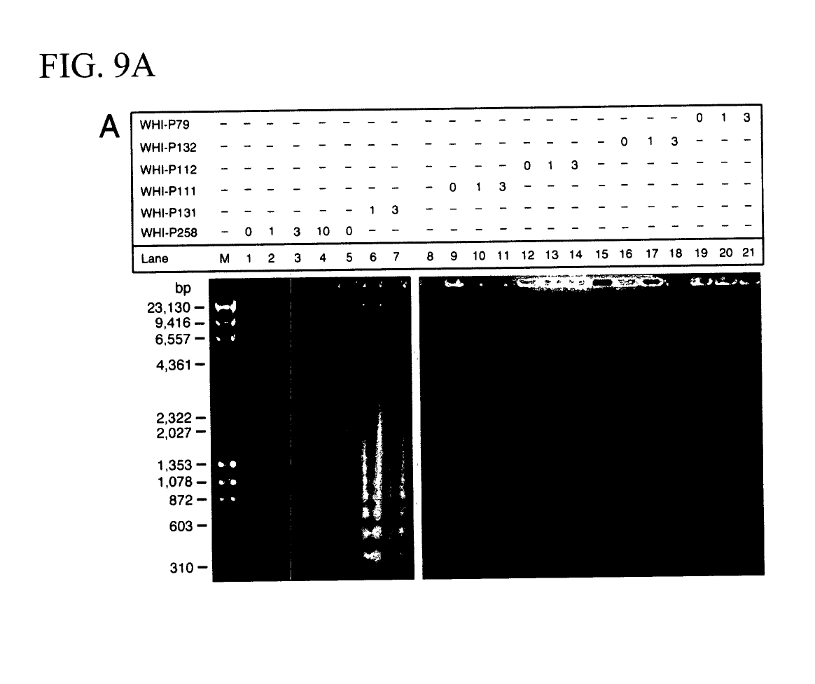

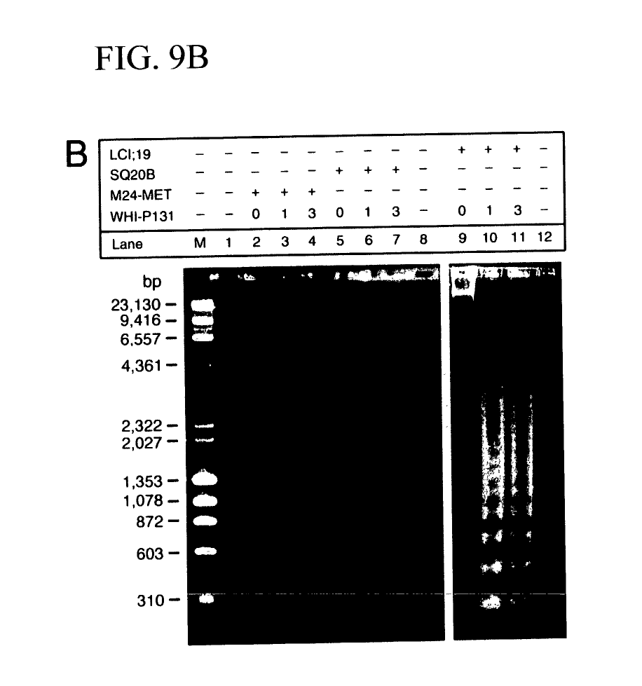

FIG. 9A demonstrates that supernatants from NALM-6 leukemia cells, treated with 1 μM or 3 μM COMPOUND 6, contained oligonucleosome-length DNA fragments with a “ladder-like” fragmentation pattern consistent with apoptosis, whereas no DNA fragments were detected in supernatants of NALM-6 cells treated with structurally similar dimethoxyquinazoline compounds which lacked JAK3 inhibitory activity. Unlike JAK3-positive leukemia cells (NALM-6 cells in FIG. 9A and LC 1; 19 cells in FIG. 9B), JAK3 negative SQ20B squamous carcinoma cells and M24-MET melanoma cells did not show any evidence of apoptotic DNA fragmentation after treatment with compound 6 (FIG. 9B).

Taken together, these results provided experimental evidence that the JAK3 specific tyrosine kinase inhibitor compound 6 results in depolarization of the mitochondrial membrane and triggers apoptotic death in human B-lineage ALL cells, as evidenced by the ladder-like fragmentation pattern of nuclear DNA and digoxigenin-11-UTP labeling of the exposed 3′-hydroxyl end of the fragmented nuclear DNA in the presence of TdT.

Clonogenic Assays

The antileukemic activity of compound 6 against clonogenic tumor cells was examined using a methylcellulose colony assay system (Uckun, F. M., et al., J. Exp. Med. 163: 347-368, 1986; Messinger, Y., et al., Clin Cancer Res. 4: 165-70, 1998). In brief, cells (10⁵/ml in RPMI-10% FBS) were treated overnight at 37° C. with compound 6 at varying concentrations. After treatment, cells were washed twice, plated at 10⁴ or 10⁵ cells/ml in RPMI-10% FMS-0.9% methylcellulose in Petri dishes, and cultured for 7 days at 37° C. in a humidified 5% CO₂ incubator. Subsequently, leukemic cell (or tumor cell) colonies were enumerated using an inverted phase-contrast microscope. The percent inhibition of colony formation was calculated using the following formula:

$$\% Inhibition = 1 - \frac{mena number of colonies in test culture}{mean number of colonies in control culture} \times 100$$

The antileukemic activity of compound 6 was measured by determining its ability to inhibit the in vitro clonogenic growth of the ALL cell lines NALM-6, DAUDI, LC1; 19, RAMOS, MOLT-3, and the AML cell line HL-60. As detailed in Table 2, compound 6 inhibited clonogenic growth in a concentration-dependent fashion with EC₅₀ values of 24.4 μM for NALM-6 cells and 18.8 M for DAUDI cells. At 100 μM, compound 6 inhibited the in vitro colony formation by these leukemia cell lines by >99%. In contrast, compound 6 did not inhibit the clonogenic growth of JAK3-negative M24-MET melanoma or SQ20B squamous carcinoma cell lines

TABLE 2
Effects of Compound 6 Against Clonogenic Leukemic Cells.

| Experiment | (6) | | | | |
|---|---|---|---|---|---|
| Number | Concn. (μM) | Cell Lines* | | | |
| | | NALM-6 (pre-B ALL) | | BT20 (Breast Cancer) | |
| | | Mean No. of | | Mean No. of | |
| | | Colonies/10⁵ Cells | % Inhibition | Colonies/10⁵ Cells | % Inhibition |
| 1 | 0 | 2890 (2660, 3120) | N.A. | 2676 (2712, 2640) | N.A. |
| | 0.1 | 2970 (2756, 3184) | 0 | N.D. | N.D. |
| | 1 | 3180 (3080, 3136) | 0 | N.D. | N.D. |
| | 10 | 1932 (1864, 2000) | 33.2 | 3298 (2940, 3656) | 0 |
| | 100 | 2 (2, 2) | >99.9 | 2190 (1632, 2748) | 18.2 |
| | | DAUDI (B-ALL) | | M24-MET (Melanoma) | |
| | | Mean No. of | | Mean No. of | |
| | | Colonies/10⁵ Cells | % Inhibition | Colonies/10⁵ Cells | % Inhibition |
| 2 | 0 | 3950 (3100, 4800) | N.A. | 177 (120, 235) | N.A. |
| | 0.3 | 2030 (1700, 2360) | 48.6 | 312 (238, 386) | 0 |
| | 1 | 2136 (1568, 2704) | 45.9 | 287 (157, 418) | 0 |
| | 3 | 1406 (988, 1824) | 64.4 | 390 (280, 500) | 0 |
| | 10 | 1149 (1054, 1244) | 70.9 | 301 (249, 353) | 0 |
| | 30 | 29 (12, 46) | 98.0 | 599 (534, 664) | 0 |
| | | RAMOS (B-ALL) | | SQ20B (Squamous Carcinoma) | |
| | | Mean No. of | | Mean No. of | |
| | | Colonies/10⁵ Cells | % Inhibition | Colonies/10⁵ Cells | % Inhibition |
| 3 | 0 | 1286 (1164, 1408) | N.A. | 754 (452, 1056) | N.A. |
| | 30 | 2 (0, 4) | 99.8 | 838 (600, 1076) | 0 |
| | | MOLT-3 (T-ALL) | | HL-60 (AML) | |
| | | Mean No. of | | Mean No. of | |
| | | Colonies/10⁵ Cells | % Inhibition | Colonies/10⁵ Cells | % Inhibition |
| | 0 | 1322 (1252, 1392) | N.A. | 1854 (1648, 2060) | N.A. |
| | 100 | 1 (1, 1) | >99.9 | 1 (1, 1) | >99.9 |

*Cells were treated with COMPOUND 6 and then assayed for colony formation as described in Methods.
N. A. = not applicable,
N. D. = not determined

In other studies, compound 6 was shown to inhibit JAK3, but not other protein tyrosine kinases, including JAK2, SYK, BTK, LYN, and IRK. ALL cells express JAK2. Similarly, the Src family PTK LYN, Zap/Syk family PTK SYK, and Tec family PTK BTK are expressed in ALL cells and affect their adhesion, proliferation, and survival (Vassilev, A., et al., J. Biol. Chem. 274. 1646-1656, 1999; Uckun, F. M., et al., Science. 267. 886-91, 1995; Kristupaitis, D., et al., J. Biol. Chem. 273 (15): 9119-9123, 1998; and Xiao, J., et al., J. Biol. Chem. 271: 7659-64, 1996). IRK is the only member of the receptor PTK family that has been detected in leukemic cells, especially pre-B ALL cells with a t(1;19) translocation (41-43). Since compound 6 does not inhibit these tyrosine kinases, its ability to kill ALL cells cannot be attributed to a nonspecific inhibition of JAK2, LYN, SYK, BTK, or IRK in these cells (Kaplan, G. C., et al., Biochem. Biophys. Res. Commun. 159(3): 1275-82, 1989; Newman, J. D., et al., Int. J. Cancer. 50(3): 500-4, 1992; Bushkin, I. and Zick, Y., Biochem. Biophys. Res. Commun. 172(2): 676-82, 1990).

The above shows that a representative JAK-3 inhibitor of formula I (Compound 6) is a useful therapeutic agent for treating acute lymphoblastic leukemia, and demonstrates that compound 6 triggers apoptosis in leukemia cells. Thus, potent and specific inhibitors of JAK3, such as dimethoxyquinazolines of formula I, are useful for treating acute lymphoblastic leukemia, which is the most common form of childhood cancer.

### Example 4

### Skin Cancer Assays

The ability of a compound to prevent or treat skin cancer can be determined using assays that are known in the art, or can be determined using assays similar to those described in this example.

Female, 6-7 weeks old, hairless albino mice (skh-1) were purchased from Charles River Laboratories (Wilmington, Mass.) and were housed in a controlled environment (12-h light/12-h dark photo period, 22±1° C., 60±10% relative humidity), which is fully accredited by the USDA (United States Department of Agriculture). Animals were caged in groups of five in a pathogen free environment in accordance with the rules and regulations of U.S. Animal Welfare Act, and National Institutes of Health (NIH). All mice were housed in microisolator cages (Lab Products, Inc., NJ) containing autoclaved bedding. The mice were allowed free access to autoclaved pellet food and tap water throughout the experiments. Animal care and the experimental procedures were carried out in agreement with institutional guidelines.

Buffered formalin phosphate (10%) was obtained from Fisher scientific (Springfield, N.J.). Dimethyl sulfoxide (DMSO) and Phosphate buffered saline (PBS) were purchased from Sigma (St. Louis, Mo.).

Ultraviolet lamps (8-FSX24T 12/HO/UVB) that emit light predominantly in the UVB range (280-320 nm) were obtained from National Biological Corporation, Twinsburg, Ohio. The irradiance of the UVB lamps was determined before each irradiation, using a UVB meter (model—500C obtained from National Biological Corporation, Twinsburg, Ohio). For exposure to UV light, three mice were placed in an open 28 cm×10 cm plastic box that was divided in three compartments (one mouse per compartment). The plastic box was placed in the center, under the bank of UVB light, and the mice were irradiated with 35 mj/cm² dose of UVB. The exposure time for a 35 mj/cm² dose of UVB varied from 30-34 s. The distance between UVB lamps and the surface receiving irradiation was 20 cm.

Tumorigenesis Protocol:

Mice were divided into three groups containing 5-14 mice per group as shown in Table 3. The mice were treated topically with a single application of Compound 6 (1.0 mg/cm², dissolved in DMSO) over a 2 cm² area on the dorsal surface before each UVB exposure. After 15 min. following Compound 6 application the mice were irradiated with UVB (35 mj/cm²). UVB exposure of mice was performed three times a week for a total of 20 weeks. Control mice were treated with vehicle prior to UVB light exposure.

TABLE 3
Experimental Design and Treatment
Regimen

| | | | UVB |
|---|---|---|---|
| Group | No. of Mice | Treatment | (35 mj/cm²) |
| A | 5 | Vehicle | − |
| B | 10 | Vehicle | + |
| C | 14 | Compound 6 (1 mg/cm²) | + |

The skin thickness of mice, and papillary skin lesions greater than 1 mm in diameter were measured and recorded twice every week and the average of the two measurements was used in the calculations. Skin thickness, lesion number and lesion diameter measurements were limited only to the 2 cm2 area on the dorsal surface of mice that was being treated either with compound 6 or vehicle. Lesion volume were calculated using the formula:

Volume=4/3πr³

At the end of the study, 5-19 lesions from each group were randomly biopsied, and fixed in 10% buffered formalin. Formalin-fixed specimens were embedded in paraffin blocks, sectioned at 4 μm thickness, and stained in haematoxylin-eosin. The pathological evaluation of skin sections was performed by a Certified Veterinary Pathologist who was unaware of the identity of specimens.

Pathological Classification of Tumors:

The pathological classification of tumors was as follows: 1) Cutaneous papilloma: a tumor papillomatous growth of acanthotic epidermis without invasive growth of tumor cells into the dermis. Tumor cells do not appear a typical. 2) Actinic keratosis: a hyperplastic, orthokeratotic, mildly to moderately acanthotic epithelium. 3) Florid actinic keratosis: actinic keratosis with mild to moderately acanthotic ridges extending into the superficial dermis, resembling superficially invasive squamous cell carcinoma. 4) Keratoacanthoma: a papillary growth with a central keratin filled crater surrounded by hyperplastic, acanthotic stratified squamous epithelium. The leading edge of the tumor pushes into the underlying dermis. 5) Squamous cell carcinoma (SCC): a tumor with a typical cell nests invading into superficial and mid dermis.

Results for Example 4.

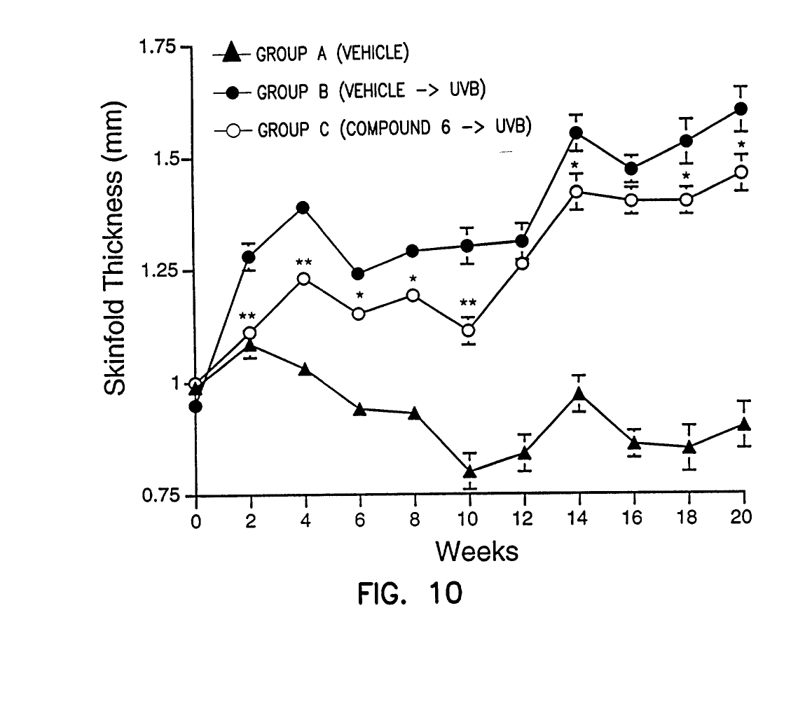

Sunburn is a UV induced inflammatory reaction that is characterized by cutaneous vasodilatation (erythema), and an increase in vascular permeability with exudation of fluid (edema) in the affected skin. The UVB-induced increase in plasma exudation can be detected as an increase in skinfold thickness at 24 h following irradiation (Berg, R. J., et al., 1998, J Invest Dermatol 110:405-9). Exposure of mice to UVB light (group B) induced an increase in skinfold thickness as compared to the skin thickness of control group mice (FIG. 10). In the compound 6 treated group (group C) there was an increase in skin thickness as compared to the control (group A); however, the increase in skin thickness was significantly less as compared to UVB-irradiated and vehicle-treated group of mice indicating the anti-inflammatory effect of compound 6. Since the mice were irradiated chronically three times a week, a sustained increase in skin edema was observed during the course of the study in UVB-irradiated mice (groups B & C). However, at any given time point the increase in skin edema was partially inhibited by compound 6 in the drug treated mice.

Chronic exposure of skin to UVB radiations primarily leads to the development of fine skin lesions that can grow both in number as well as size with further exposures to such radiations. As shown in FIG. 11, the chronic UVB exposure of mice induced the appearance of fine lesions in the affected skin that first appeared around 10 weeks of irradiation in the vehicle-treated and UVB-irradiated group of mice (group B). The total number of skin lesions increased exponentially with increasing numbers of UVB-exposure in the UVB-irradiated group of mice (group B). However, in COMPOUND 6-treated mice (group C), the onset of lesions was delayed; the first lesion was observed after 14 weeks of UVB-irradiation. Although the average number of lesions per mouse in this group also increased with increasing numbers of UVB exposures as in group B, at any given time point, the average number of lesions per mouse was always less as compared to its untreated control (group B).

This data indicate that application of compound 6 prior to UV exposure (a) delays the onset of tumor from 10 weeks of UVB treatment (group B) to 14 weeks, and (b) it inhibits the total number of lesions resulting from repeated UVB exposure.

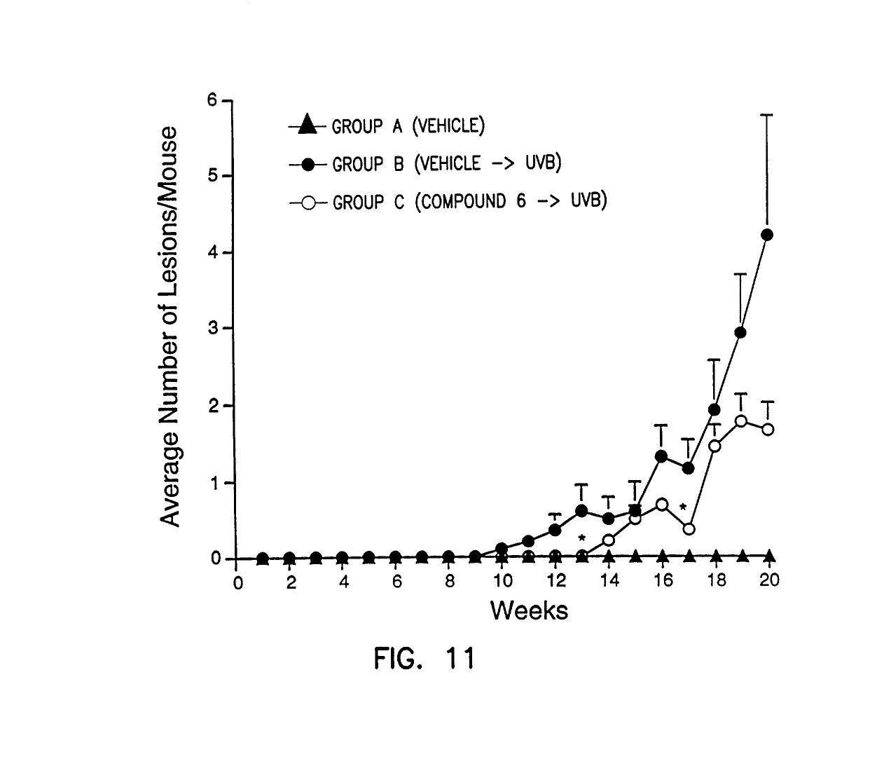

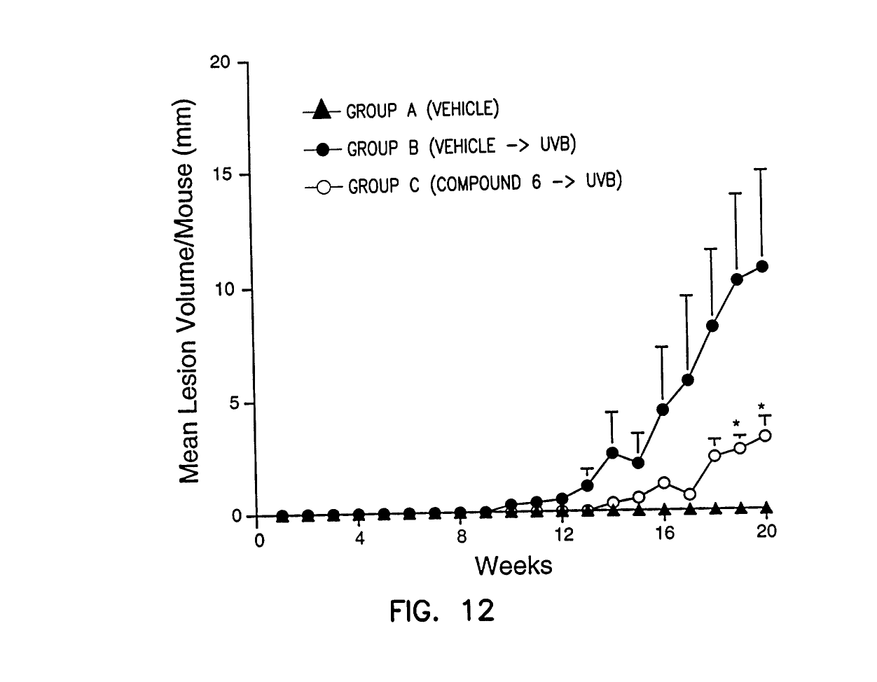

Following the first appearance around 10-14 weeks of UVB irradiation, the skin lesions increased both in total number as well as in size with increasing number of UVB exposures. In order to compare the size of skin lesions, lesion diameter was converted to lesion volume using the formula described in Materials and Methods, and average lesion volume per mouse was compared. FIG. 12 shows the effect of compound 6 treatment on average lesion volume per mouse. It is observed from the figure that the average lesion volume increased with time in both of the UVB-irradiated groups of mice (groups B & C) but compound 6 treatment of mice inhibited the increase in lesion volume (group C, FIG. 12 and Table 4). In addition, we also compared the average volume per lesion in compound 6-treated and -untreated groups of mice after 20 weeks of irradiation. As observed before, with average skin lesion volume per mouse, the average volume per lesion was also inhibited by compound 6 treatment (Table 4).

TABLE 4
Inhibitory effect of topical administration of Compound 6
on UVB-induced tumorigenesis in skh-1 mice

| | | No. of lesions/ | Skin lesion volume/ | Avg. Vol/ |
|---|---|---|---|---|
| Group | No of mice | mouse | mouse | lesion |
| A | 5 | 0 | 0 | 0 |
| B | 10 | 4.2 ± 1.6 | 10.6 ± 4.3 | 2.5 ± 0.5 |
| C | 14 | 1.6 ± 0.4 | 3.2 ± 0.9* | 1.9 ± 0.5 |

Female skh-1 mice (7-8 weeks old) were treated topically either with compound 6 (1 mg/cm²) or vehicle before each UVB irradiation (Irradiation Dose: 35 mj/cm²). The mice were exposed to UVB three times a week for 20 weeks. The data represent the mean ± SEM from 5-14 mice at 20 weeks.
*P < 0.05 as compared to vehicle treated control.

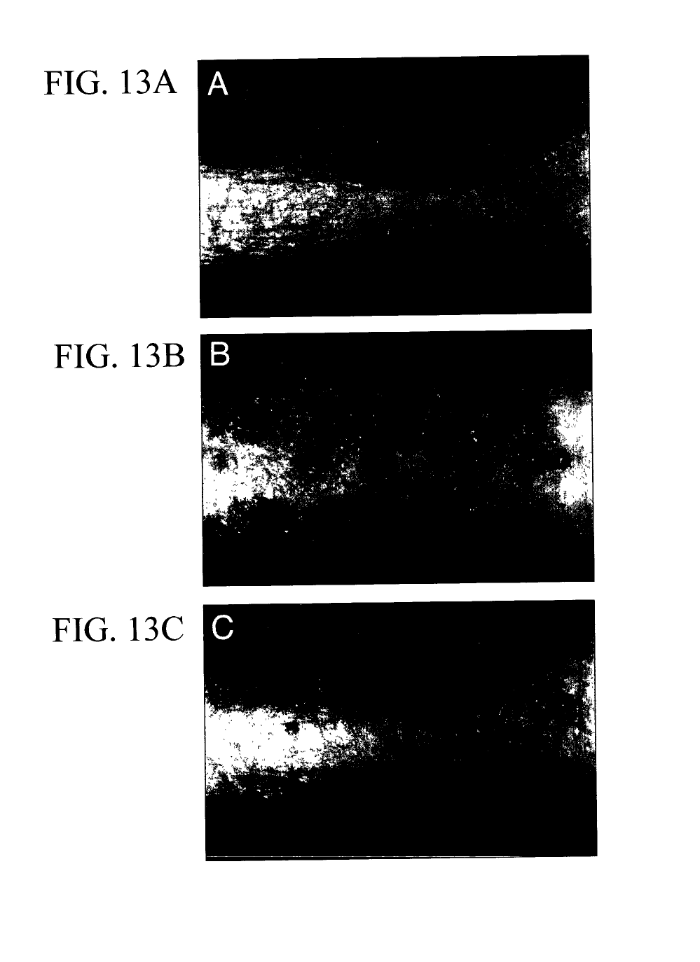

Morphological and Histopathological Data. The morphological appearance of mouse skin after 20 weeks of irradiation from all three groups of mice has been compared. The FIG. 13 shows that repeated exposure with UVB light induced the growth of a number of lesions on the affected skin (FIG. 13, Panel B) whereas treatment with compound 6 inhibited the growth of such lesions.

Histopathological findings of skin biopsies in mice after 20 weeks of UVB irradiation are presented in Table 5.

TABLE 5
Inhibitory effect of topical administration of
Compound 6 on UVB-induced keratoacanthoma
and squamous cell carcinomas in skh-1 mice

| | | Percentage | Percentage | | |
|---|---|---|---|---|---|
| | | of mice | of mice | Percentage | Percentage of |
| | | with | with | of mice | mice with SCC / |
| | No of | Actinic | Keratoaca- | with | Florid actinic |
| Group | mice | keratosis | nthomas | Papilloma | keratosis |
| A | 5 | 0 | 0 | 0 | 0 |
| B | 10 | 60 | 10 | 10 | 80 |
| C | 14 | 53 | 0 | 7 | 57 |

Female skh-1 mice (7-8 weeks old) were treated topically either with compound 6 (1 mg/cm²) or vehicle before each UVB irradiation (Irradiation Dose: 35 mj/cm²). The mice were exposed to UVB three times a week for 20 weeks. The data represent the percentage of total no. of mice per group at 20 weeks.

A mild to moderate dermatitis was observed in all three groups including the unirradiated group of mice (group A) which could be induced by topical application of the vehicle (DMSO) three times a week during the course of the study. Actinic keratosis, which represents a hyperplastic and mild to moderately acanthotic epidermis, was present in most of the biopsies from both of the UVB irradiated groups (groups B &C). A single lesion of keratoacanthoma was observed in the UVB-irradiated and vehicle treated group of mice (group B). No such lesion was observed in any of the 14 mice in compound 6 treated group (group C). The occurrence of superficially invasive squamous cell carcinoma (Florid actinic keratosis and a precursor to SCC) and SCC was noted in both of the UVB irradiated groups (Group B &C), but in COMPOUND 6 treated mice (group C) it was inhibited by about 23% (Table 5). Similarly, the incidence of cutaneous papilloma was also partially inhibited by compound 6.

The results of Example 4 indicate that the topical application of compound 6 prior to UV irradiation protects skin from the harmful consequences of skin cancer in chronic exposure. Specifically, a topical application of compound 6 on skin before UVB light exposure markedly inhibited the formation of skin lesions, decreased tumor size and inhibited the development of tumors in skh-1 mice. Extensive documentation has validated the role of UVB radiations in skin tumor formation (Devary, Y., et al., 1992, Cell 71.1081-91; Ley, R. D., et al., 1989, Photochem Photobiol 50:1-5; Hall, E. J., et al., 1988, Am J Clin Oncol 11:220-52; and Marks, R. 1995, Cancer 75:607-12). UVB-irradiation of skin cells triggers the release of increased amounts of arachidonic acid and its metabolites (Konger, R. L., et al., 1998, Biochim BiophysActa 1401:221-34. (12-15). Prostaglandins, which are the oxygenation product of arachidonic acid, are produced abundantly following UVB irradiation of skin cells (Hawk, J. L. M., and J. A. Parrish. 1993. Responses of Normal Skin to Ultraviolet Radiations. Plenum Medical Book Publishers, New York; Hruza, L. L., and A. P. Pentland. 1993, J Invest Dermatol 100:35S-41S; Kang-Rotondo, et al., 1993, Am J Physiol 264:C396-401; and Grewe, M., U. et al., 1993, J Invest Dermatol 101:528-31) and have been implicated in various models for tumorigenesis (Vanderveen, E. E., et al., 1986, Arch Dermatol 122:407-12; Cerutti, P. A., and B. F. Trump. 1991, Cancer Cells 3:1-7). Evidences also indicate that in addition to stimulating tumor growth, prostaglandins have a tendency to suppress hosts' immune surveillance (Plescia, O. J., et al., 1975, Proc Natl Acad Sci USA 72:1848-51; Goodwin, J. S. 1984. Am J Med 77:7-15 and thus, assist in tumor promotion. Elevated levels of PGE₂ have been observed in squamous and basal cell carcinomas of skin and may be correlated with the increased metastatic activity and invasive behavior (Vanderveen, E. E., et al., 1986, Arch Dermatol 122:407-12; Klapan, I., V. et al., 1992, J Cancer Res Clin Oncol 118:308-13 of these tumors. Various chemical compounds which inhibit the production of prostaglandins have been observed to inhibit the growth of tumors (Snyderman, C. H., et al., 1995, Arch Otolaryngol Head Neck Surg 121:1017-20; Hial, V., et al., 1976, Eur J Pharmacol 37:367-76; Lynch, N. R., et al., 1978, Br J Cancer 38:503-12.

The ability of compound 6 to inhibit the UVB induced skin tumorigenesis combined with our previous observations that it inhibits acute skin inflammation indicate that compound 6 is a useful chemopreventive agent against some forms of human cancers induced by environmental agents, such as ultraviolet light.

### Example 5

### UVB-Induced Skin Carcinogenesis Assays

The ability of a compound to prevent or treat skin cancer can be determined using assays that are known in the art, or can be determined using assays similar to those described in this example.

Compound 6 is able to significantly inhibit the UVB light induced inflammatory mediator release in epidermal cells and thus it prevents the inflammatory responses of UVB exposed skin.

Female, 6-7 weeks old, hairless albino mice (skh-1) were purchased from Charles River Laboratories (Wilmington, Mass.). The transgenic BigBlue mice carrying multiple copies of the BigBlue (LIZ shuttle vector which contains substrate for detection of mutations in vivo were obtained from Stratagene (La Jolla, Calif.). Mice were caged in groups of five in a pathogen free environment in accordance with the rules and regulations of U.S. Animal Welfare Act, and National Institutes of Health (N1H). Animal care and the experimental procedures were carried out in agreement with institutional guidelines.

HaCaT, which is a spontaneously transformed human epidermal cell line (16) was maintained in Dulbecco's modified Eagle's medium (DMEM) supplemented with 10% fetal bovine serum (Summit Biotech, Ft. Collins, Colo.).

A bank of 8-FSX24T12/HO/UVB lamps (National Biological Corporation, Twinsburg, Ohio) that emits light predominantly in the UVB range, 280-320 nm was used to irradiate mice and cell cultures. The irradiance of UVB lamps was always measured before irradiation using a UVB meter (model—500C obtained from National Biological Corporation, Twinsburg, Ohio). The dose of UVB light used to irradiate cultures was 25 mj/cm². Anesthetized mice received UVB radiations to a 2.0 cm² area on their dorsal surface. This area was painted either with the test compound (1.5 mg/cm²) in drug receiving mice, or with vehicle in control mice fifteen minutes before irradiation. The distance between UVB lamps and surface receiving irradiation was 20 cm. The final radiation dose received by skh-1 hairless mice was 250 mj/cm² which is approximately seven times higher than the minimal erythema dose (MED) (17) in these mice. BigBlue mice were shaved on their back before irradiation and then exposed to 400 mj/cm² UVB light either in the presence or absence of compound 6.

Prostaglandin E₂ Assay.

Confluent HaCaT cells cultured in 24-well culture dishes were washed three times with serum-free DMEM containing 1% bovine serum albumin (BSA) (Cayman chemicals, Ann Arbor, Mich.) and incubated with 3-30 μM compound compound 6 for 1 hour at 37° C. After incubation the cells were washed twice with PBS, and either exposed to 25 mj/cm² UVB light or stimulated with 50 ng/ml rhEGF, and fed with serum-free DMEM containing 1% BSA. compound 6 was readministered and the cells were incubated for 6 h at 37° C. At 6 h following stimulation the cell supernatant was collected and PGE₂ released in cell supernatants was measured by a competitive EIA using an acetylcholine esterase-PGE₂ tracer and anti-PGE₂ antibody supplied with the ELISA kit (Cayman chemicals, Ann Arbor, Mich.). Cellular protein was determined using the Pierce's BCA protein assay method (Smith, P. K., et al., 1985, Anal Biochem. 150 (1):76-85).

Compound 6 was initially dissolved in dimethyl sulfoxide (DMSO) (Sigma, St. Louis, Mo.) at a concentration of 10 mg/ml and diluted to 1 mg/ml concentration with Phosphate buffer saline (PBS) before injection. The mice were treated daily with 16 mg/kg i.p. bolus injection of compound 6 from day −2 (i.e. 2 days prior to UVB exposure) unlil the termination of the experiment. Drug receiving mice were also painted with 1.5 mg/cm² of Compound 6, 15 min before irradiation. The control mice received vehicle alone.

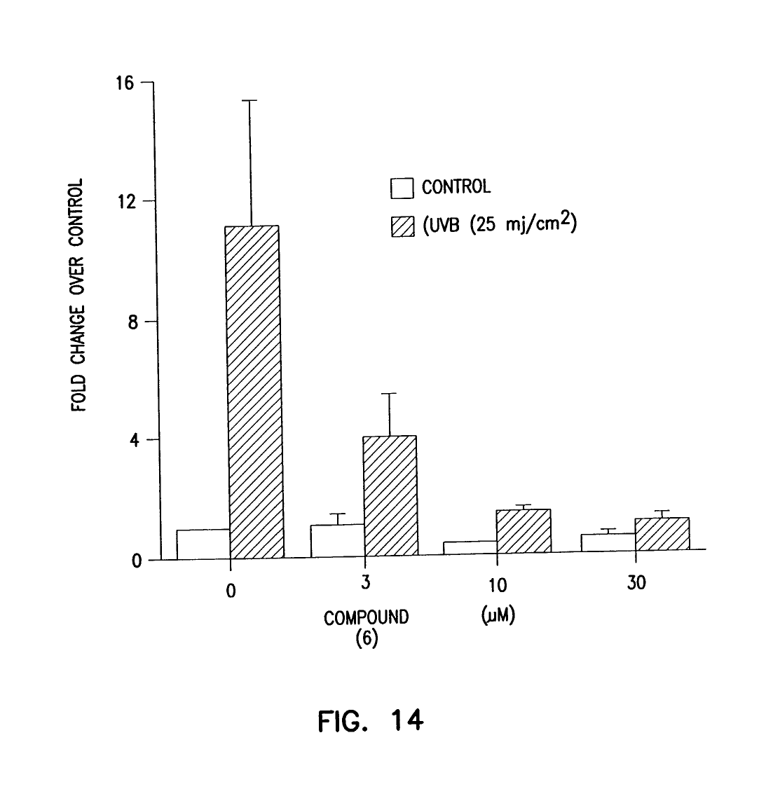

One of the major arachidonic acid metabolite in Ultraviolet light B-irradiated keratinocytes is prostaglandin E₂ which can be detected as early as 6 h, peaks between 24-48 h following UVB exposure, and induces edema and erythema in skin (Gilchrest, B. A., et al., 1981, J Am Acad Dermatol. 5 (4):411-22; Konger R. L., et al., 1998, Biochim. et Biophys. Acta (1401):221-34; Woodward, D. F., et al., 1981, Agents Actions. 11 (6-7):711-7; Snyder, D. S., and W. H. Eaglstein. 1974. Br J. Dermatol. 90 (1):91-93; Snyder, D. S., and W. Eaglstein. 1974. J Invest Dermatol. 62:47-50; and Gupta, N., and L. Levy. 1973, Br J. Pharmacol. 47 (2):240-8). We determined the effect of compound Compound 6 on PGE₂ release in UVB irradiated epidermal cells. The human epidermal cells, HaCaT, were exposed to UVB light and incubated both in the presence or absence of Compound 6 and cumulative PGE₂ released in cell supernatants during 6 h incubation was determined. Analysis of PGE₂ release (FIG. 14) showed that an exposure of HaCaTs to 25 mj/cm² UVB induced about eleven (11.11 (4.23) fold increase in PGE₂ level at 6 h post irradiation as compared to non-UVB irradiated control. The UVB light induced prostaglandin release was inhibited by Compound 6 in a concentration dependent manner and about 90% inhibition in prostaglandin release was observed at 30 μM dose of Compound 6. This data indicated that Compound 6 is able to inhibit prostaglandin E₂ release in UVB light-stimulated epidermal cells.

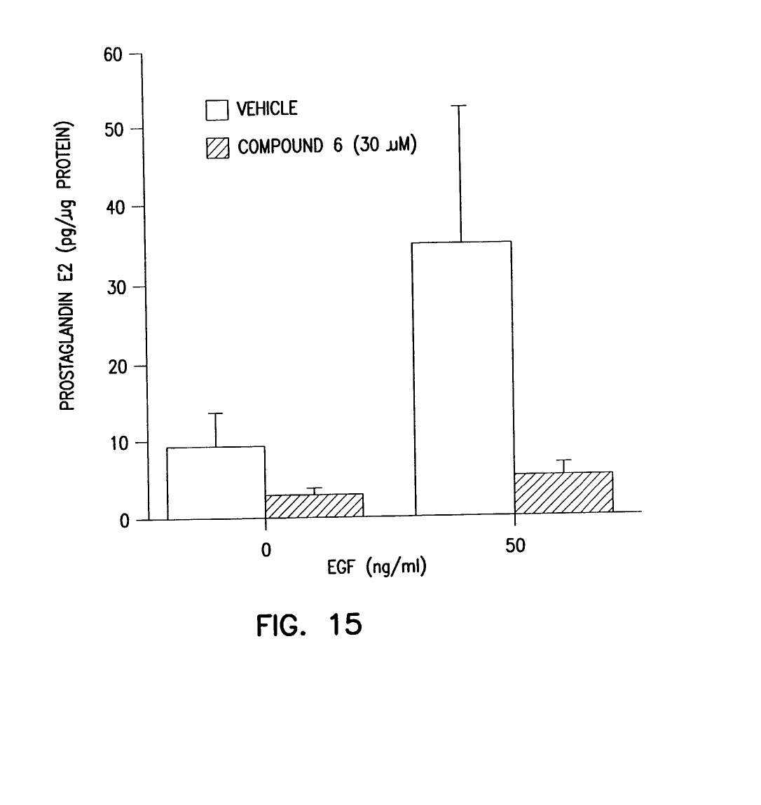

Primary human keratinocytes as well as HaCaTs express significantly high number of functional EGF-receptors on their cell surface ( ). Upon stimulation the EGF-receptors present on the epidermal cells participate in transmembrane signaling and induce the formation of prostaglandins. To determine that the previously observed inhibition in prostaglandin release by Compound 6 was due to the inhibition of EGF-R activation, we studied the effect of Compound 6 on EGF-stimulated prostaglandin formation in epidermal cells. Stimulation of the HaCaT cells with 50 ng/ml rhEGF for 6 h induced about 4 fold increase in prostaglandin release over unstimulated control, (FIG. 15) and the observed increase in prostaglandin release was inhibited in the presence of 30 μM Compound 6, indicating that (i) epidermal growth factor receptors mediate prostaglandin formation in epidermal cells, and (ii) Compound 6 inhibits the prostaglandin release in UVB light stimulated cells through the inhibition of EGF-R activation. Morphology and Skin edema measurement.

Morphological appearance of the UVB irradiated skin was monitored visually and compared with the control mice skin. Skinfold thickness of the dorsal surface of the mice was recorded before, and everyday after UVB exposure for five days with a digital thickness gauge (Mitutoyo, So. Plainfield, N.J.) which measures thickness in 0-10 mm range with an accuracy of 0.015 mm.

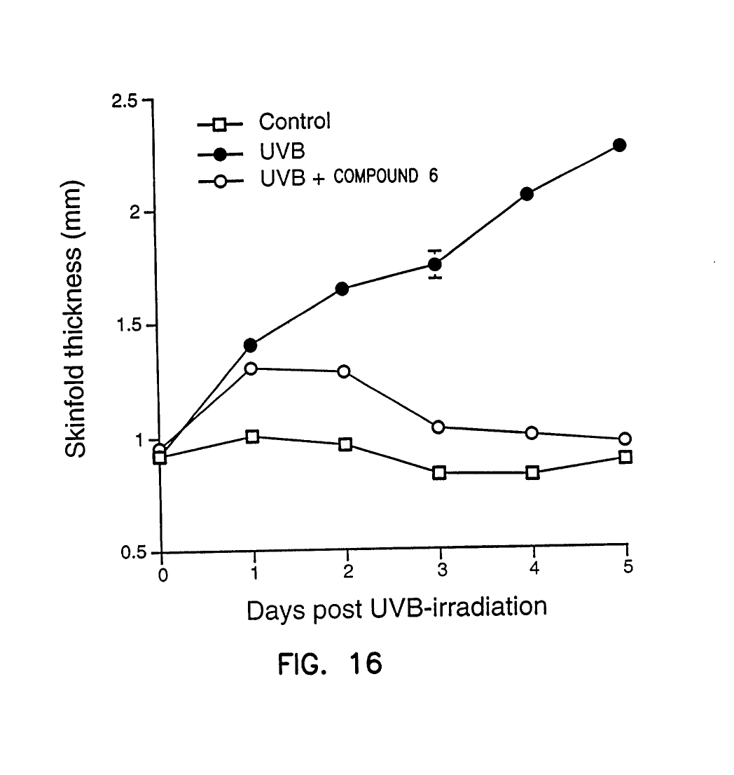

As mentioned earlier, Prostaglandin E₂ is a potent inflammatory mediator and is well known to induce vasodilation and potentate edema in skin following injury. Since our in vitro study using epidermal cells showed that compound Compound 6 inhibits the release of PGE₂ in UVB stimulated cells, we were interested to determine if Compound 6 is able to inhibit the harmful inflammatory responses of skin following UVB light exposure in vivo. For these studies we used female, hairless, albino skh-1 mice and exposed their dorsal surface with 250 mj/cm² UVB light. The skinfold thickness of the dorsal surface of the mice was determined as a measurement of skin edema from day 1 through day 5 following irradiation. FIG. 16 shows that a single exposure of mice to 250 mj/cm² UVB induced a time dependent increase in skinfold thickness. Compound 6 was not able to effectively inhibit the skin edema at 24 h post irradiation. However, it blocked further increase in skin thickness by 48 h post-UVB exposure in irradiated group of mice. In contrast, the skin thickness increased to about 70% of the control mice skin thickness at the same time point. At 72 h post irradiation, a significant decrease in skin edema was observed in Compound 6 treated mice and by day five post irradiation, the skinfold thickness in drug treated mice was almost back to control level. In contrast, in vehicle treated group the skin thickness increased to a total of 2.5 fold of control mice skin thickness over five day period. Similar results were obtained with P131 dissolved in polyethylene glycol-200 (PEG-200) (data not shown). These observations indicated that Compound 6 is able to inhibit UVB light-induced skin edema in mice.

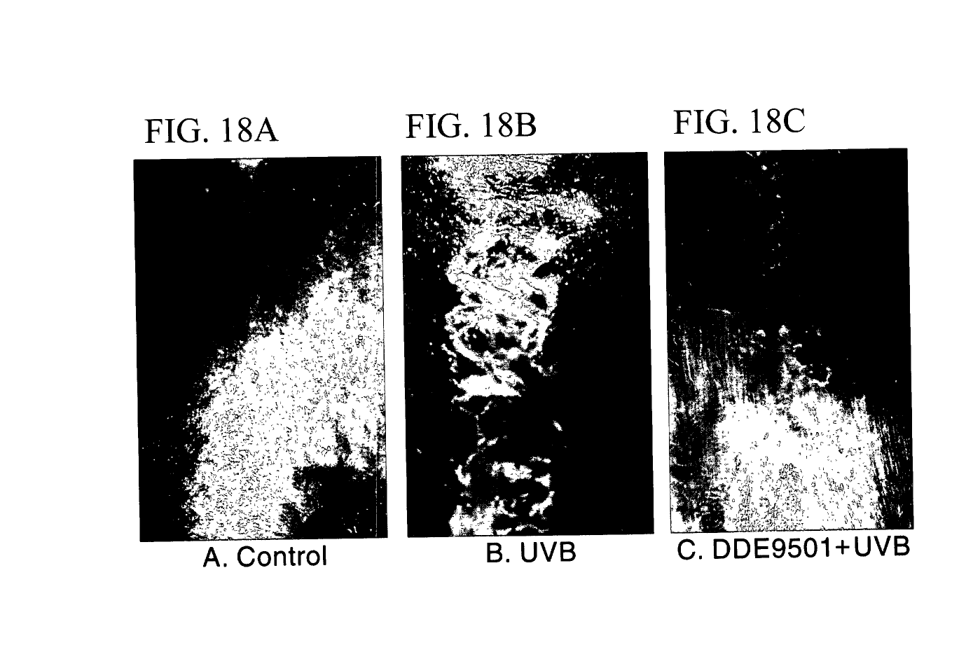

We also monitored the morphological changes in skin appearance following UVB light exposure in Compound 6 treated and vehicle treated groups of mice. Sunburn damage to the skin in first 24-48 h following UVB exposure was visible as an “elephant skin” appearance of the skin surface. The UVB light irradiated skin of mice appeared pink in color, leathery and thick. On day 1 and day 2 following the induction of inflammation, no significant difference was noted in skin appearance of drug treated and vehicle treated groups of mice. However, in UVB irradiated, vehicle treated mice, starting at day 3 many flakes of desquamating skin could be seen peeling off the skin surface and by day 5 the skin of this group of mice had become tough, leathery, and had developed scars on the surface. In contrast, in Compound 6 treated group, the skin appearance of mice improved following day 3 and by day 5 post-UVB, the signs of skin inflammation had diminished and the skin appearance resembled to that of control group mice (FIG. 18).

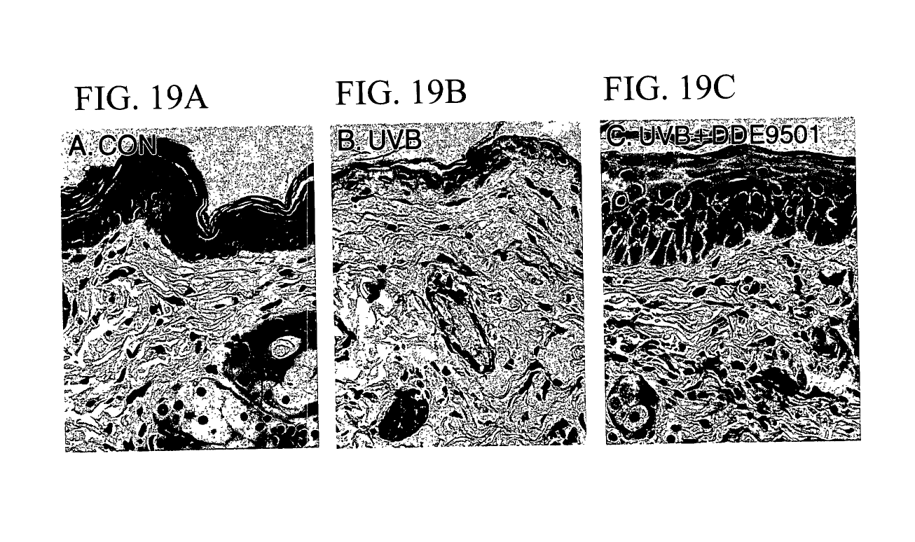

UVB light-induced histological changes of the skin were also studied. The normal epidermis typically has a 2-3 cell layer and contains scattered inflammatory cells especially around hair follicles (FIG. 19A). The UVB-irradiated skin showed thickened epidermis with 3-5 cell layers. Large number of neutrophils were also accumulated in the dermis (FIG. 19B). In contrast, the skin of mice treated with Compound 6 looked very much like the skin of unirradiated controls (FIG. 19C), with 1-2 cell layers of epidermis and normal dermis. Thus, Compound 6 prevented development of edema and neutrophil influx in UVB irradiated skin of mice.

Vascular Permeability.

Vascular permeability was quantitatively assayed by leakage from vessels of an albumin bound anionic dye, Evans blue (Sigma, St. Louis, Mo.). Evans blue (1%, 200 (1/mouse) was injected via the tail vein, 4 h later the mice were killed and the dorsal irradiated skin was biopsied. From the biopsies the dye was extracted in formamide (Sigma, St. Louis, Mo.) by warming the samples at 80° C. for 2 h and the optical absorbance of the formamide was measured at 620 nm.

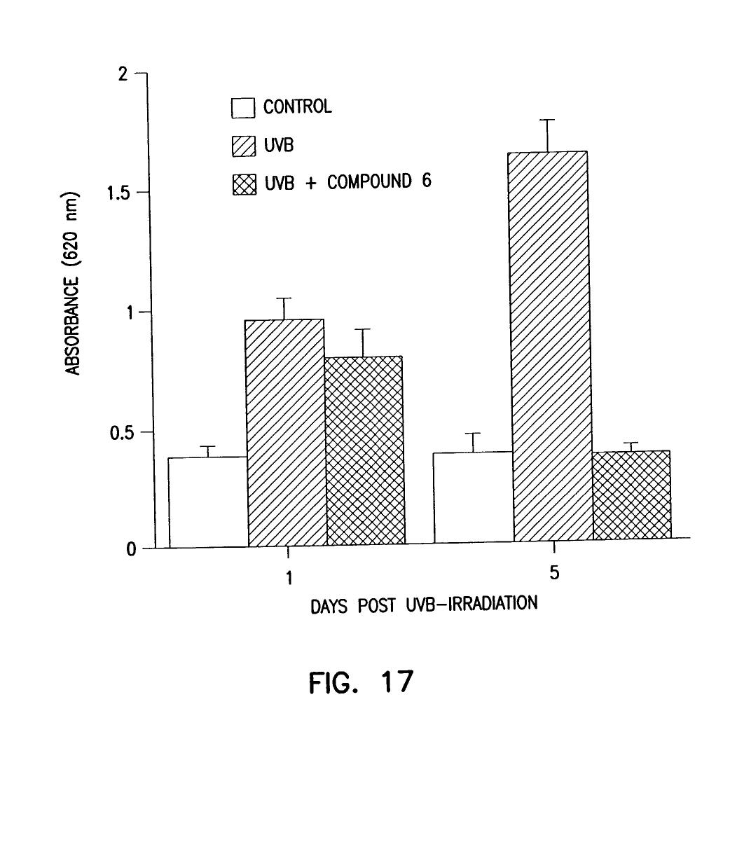

Since edema is associated with increased plasma exudation, we determined the effect of Compound 6 on UVB induced skin vascular permeability. The data on vascular permeability changes following the UVB irradiation has been presented in FIG. 17. Consistent with the skin edema findings there was an increase in vascular permeability of the skin in UVB irradiated mice. The effect of Compound 6 was minimal at 24 and 48 h post irradiation. However, at day 5 post-UVB, in Compound 6 treated group the vascular permeability was back to control level whereas a five fold increase in vascular permeability was observed in UVB exposed, vehicle treated group of mice.

Sun Burn Cell Staining and Histological Studies.

After the mice were killed by cervical dislocation, the skin was removed and spread on a sheet of dental wax. One punch (8 mm) was taken and fixed in buffered formalin. 4-5 μm thick sections were cut from paraffin blocks. Sunburn cell staining was done using ApopTag Plus In situ Detection kit (Oncor, Gaithersburg, Md.) which detects sunburn cells by direct fluorescence of digoxigenin-labelled genomic DNA. Briefly, the residues of digoxigenin-nucleotides were catalytically added to 3′-OH ends of double or single stranded DNA in presence of terminal deoxynucleotidyl transferase enzyme and the bound nucleotides were detected using anti-digoxigenin antibody conjugated with fluorescein.

To study the histological changes following irradiation the tissue sections were stained with hematoxylin and eosin and the stained slides were examined microscopically.

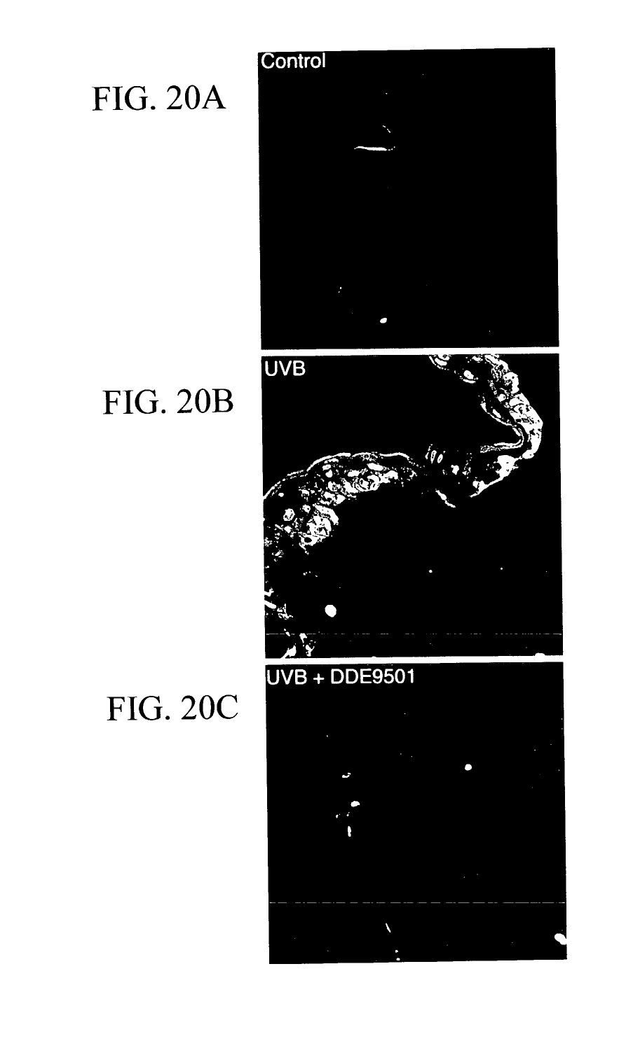

An acute exposure to UVB light induces sunburn in skin cells. The presence of sunburn cells in UVB-irradiated mice skin was detected using In Situ apoptosis detection kit as stated earlier. A significant number of sunburnt cells (FIG. 20) were observed in the skin of UVB-irradiated mice at 48 h after light exposure. In contrast, in Compound 6 treated mice significantly less or no sunburnt cells were observed at the same time point. Thus, the data suggest that Compound 6 inhibits the UVB induced cell death in mouse skin.

### Example 6

### Transplant Complications

The ability of a compound to prevent or treat transplant complications can be determined using assays that are known in the art, or can be determined using assays similar to those described in this example. Proliferation assays.

Splenocytes (4×10⁵/100 μl) from 9-wk-old C57BL6 males were used as responders in phytohemagglutinin (PHA)- and concanavalin A (Con A)-induced proliferation assays. The cells were applied in triplicates per group to a 96-well microplate in a final volume of 200 μl of RPMI 1640 medium, supplemented by 10% fetal calf serum. PHA or Con A (Sigma, St. Louis, Mo.) were added in the concentration of 5 or 2 μg/ml, respectively. Compound 6 was added in the concentration of 0.1, 1, 10 and 50 μg/ml. Cells were cultured in 5% CO₂ with humidified air in an incubator at 37° C. for 3 days. Then, a calorimetric assay for the quantification of cell proliferation, based on the cleavage of the tetrazolium salt WST-1 by mitochondrial dehydrogenases in viable cells (Boehringer Mannheim, Indianapolis, Ind.), was performed following manufacturer's instructions. The absorbance was measured at 450/690 μm on a Multiskan MS microplate scanner. The value of cell proliferation was obtained by diminishing O.D. value of PHA- or Con A-stimulated cell proliferation by O.D. value of non-stimulated cells (negative control).

Mixed Lymphocyte Reaction (MLR).

For MLR assay, responder cells (splenocytes obtained from 9-wk-old C57BL6 males) were plated in triplicates in 96-well-plates in the concentration of 4×10⁵/100 μl and mitomycin-treated stimulators (splenocytes obtained from 10-12-wk-old BALB/c males) were added in the concentration of 8×10⁴ in 50 μl. Compound 6 was added in the concentrations descried above to a final volume of 200 μl. Cells were cultured for 5 days and a colorimetric WST-1 assay was performed, as described above.

Apoptosis Detection.

C57BL6 splenocytes (3×10⁶/ml) were cultured in 24-well plate for 24 h in 500 μl of RPMI 1640 under the conditions described above. Compound 6 was added in the concentrations of 0.1, 1, 10 and 100 μg/ml. Apoptotic cell death was detected by TUNEL, using InSitu Cell Detection Kit, Fluorescein (Boehringer Mannheim, Indianapolis, Ind.). After the culture period, cells were washed, fixed, permeabilised and stained following manufacturer's instructions and apoptosis was analyzed by flow cytometry, using FACS Calibur (Becton Dickinson, San Jose, Calif.).

Mice.

Bone marrow transplant recipients were 8-10 week old C57BL/6 (H-2^{b}) male mice and donors were 6-8 week old BALB/c (H-2^{d}) males (both strains purchased at Taconic, Germantown, N.Y.). Mice were kept in Animal care facility at The Hughes Institute, under the specific-pathogen-free condition (SPF). Free access to standard mouse diet (Harlan Teklad LM-485) and water was allowed. Recipients were given antibiotic-supplemented water (sulfamethoxazole/trimethoprim, Hi-Tech Pharmacal, Amityville, N.Y.) starting the day before transplantation.

Irradiation.

Recipient mice, positioned in a pie shaped Lucite holder, were treated one day prior to bone marrow transplantation with a lethal dose (7.5 Gy) of Cesium (JL Sheppard Labs, 47.08 rad/min).

Bone Marrow Transplantation (BMT).

Donor BALB/c bone marrow was collected into RPMI 1640 with L-glutamine (Cellgro) (Mediatech, Hendon, Va.) by flushing the shafts of the femur and tibia. At the same time, donor single cell suspension of splenocytes, eliminated from red blood cells by lysis buffer (ACK lysis buffer—0.15M NH₄Cl, 1.0M KHCO₃, 0.01MNa₂EDTA) was prepared, as well. BM cells were suspended by agitation with a pasteur pipette and separated from debris by passing through a fine pore nylon cell strainer. Red blood cells were eliminated by lysis buffer and clumps of debris were allowed to settle out. The cells were washed and were resuspended for i.v. injection via the caudal vein. The standard inoculum consisted of 25×10⁶ BM cells and 25×10⁶ splenocytes in 0.5 ml of RPMI 1640).

Graft-Versus-Host Disease (GVHD) Monitoring.

BMT recipeints were monitored daily for the onset of clinical evidence of GVHD (determined by weight loss, manifestations of skin erythema, allopecia, hunching, diarrhea) and survival during the 90-day observation period. Survival times were measured from the day of BMT (day 0). Deaths occurring within 11 days of transplantation were considered to be radiation-induced and were excluded.

Drug Treatments.

For GVHD prophylaxis—daily intraperitoneal (i.p.) injections of Compound 6 (WHI-P131), 4-(3′-hydroxyl-phenyl)-amino-6,7-dimethoxyquinazoline (WHI-P132, Compound 7), Cyclosporine (Sandimmune® Sandoz Pharmaceuticals Ltd, Basle, Switzerland), Methylprednisolone (Depo-Medrol® Pharmacia & Upjohn Company, Kalamazoo, Mich.), Methotrexate (Immunex Corporation, Seattle, Wash.) and vehicle control were administered to mice starting the day before BMT (−1) or on the day of BMT (day 0). Compound 6 was administered in a dose of 25 mg/kg/day and 60 mg/kg/day (divided in three doses), Compound 7-50 mg/kg/day day (divided in three doses), from the day 0 of BMT; cyclosporine, methylprednisolone and methotrexate were injected in a doses according the Children cancer group (CCG) protocol: 3 mg/kg/day (divided in two doses), 10 mg/kg/day (divided in two doses) and 10 mg/m²/day (once daily), respectively. The treatments with cyclosporine and methylprednisolone started the day before BMT (−1) and lasted till the end of experimental period, while methotrexate was administered on days 1, 3, 6 and 11 post BMT.

Statistical Analysis.

Statistical analysis of data obtained in proliferation assays, MLR and apoptosis data was done by Student's t-test, while survival data were analyzed by life-table methods using the Mantel-Cox test.

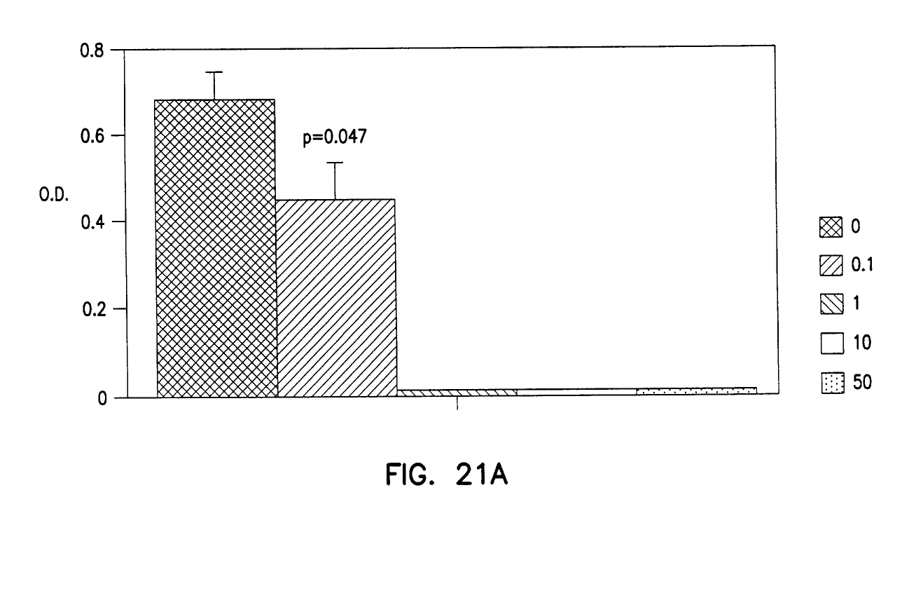

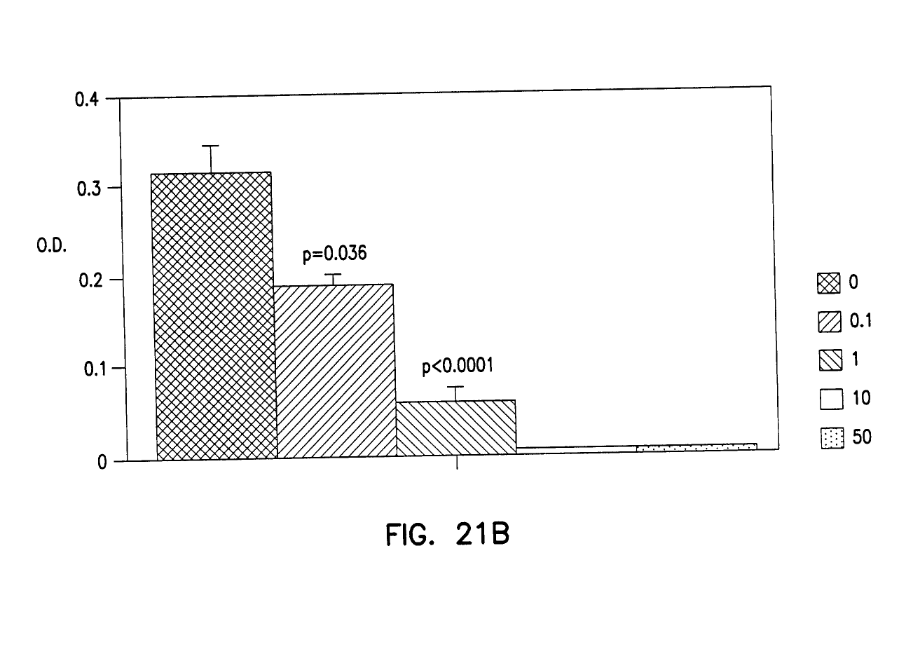

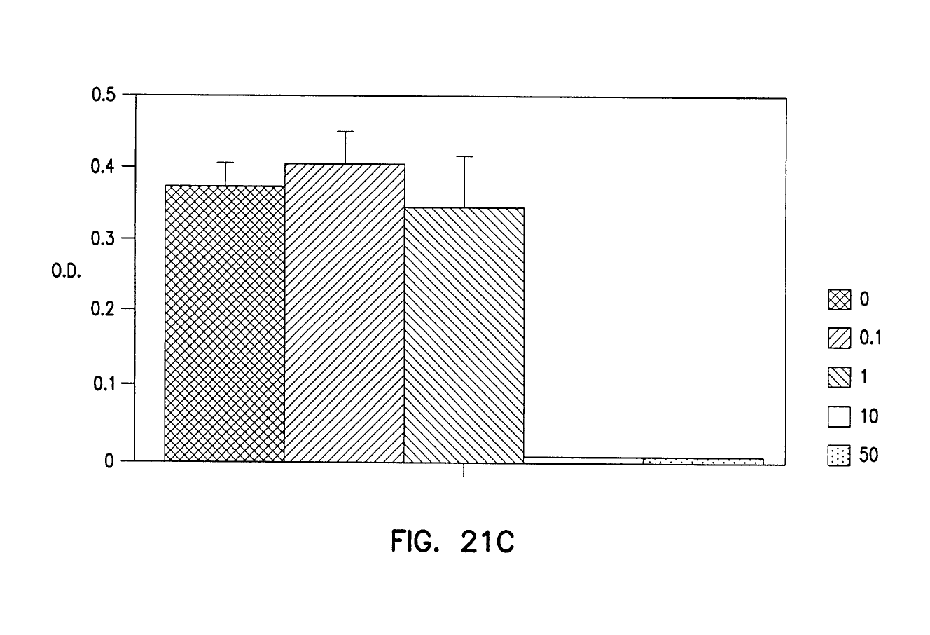

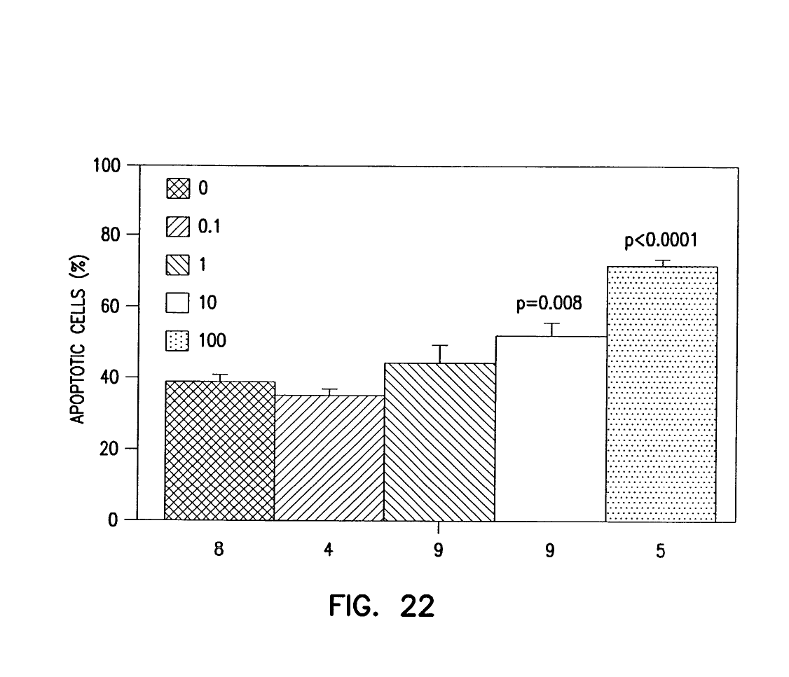

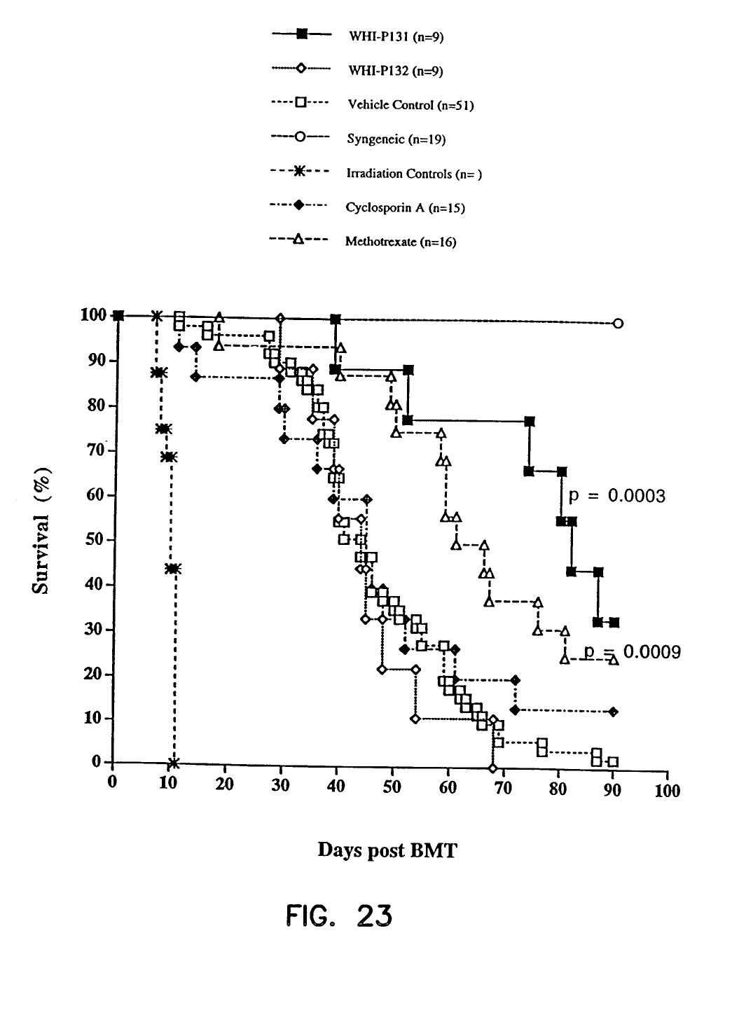

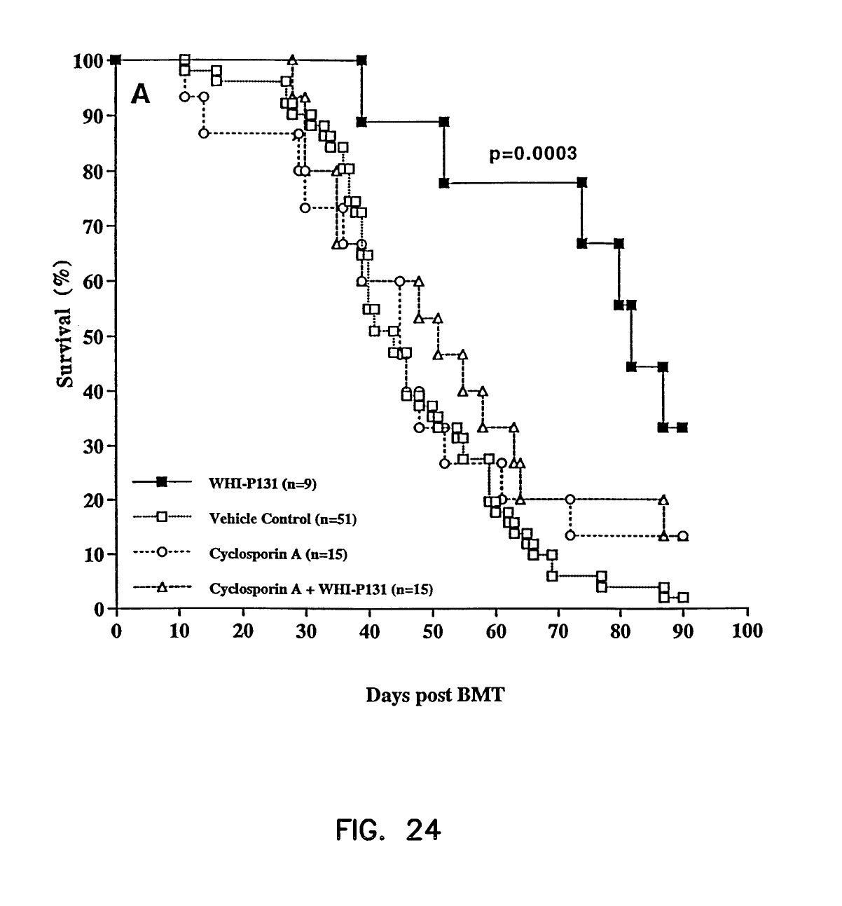

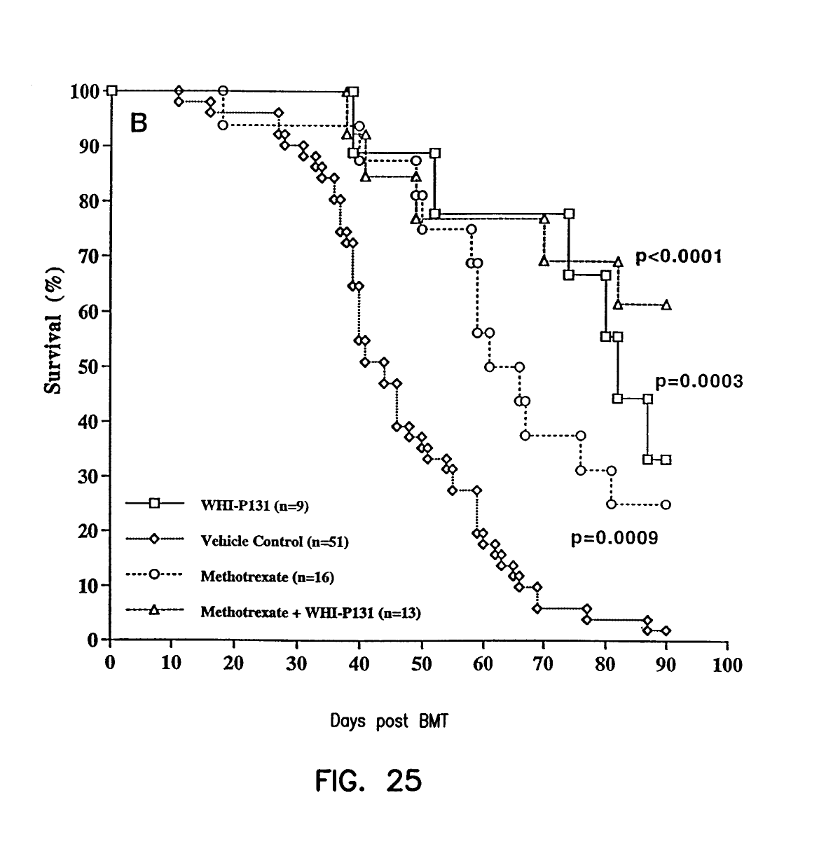

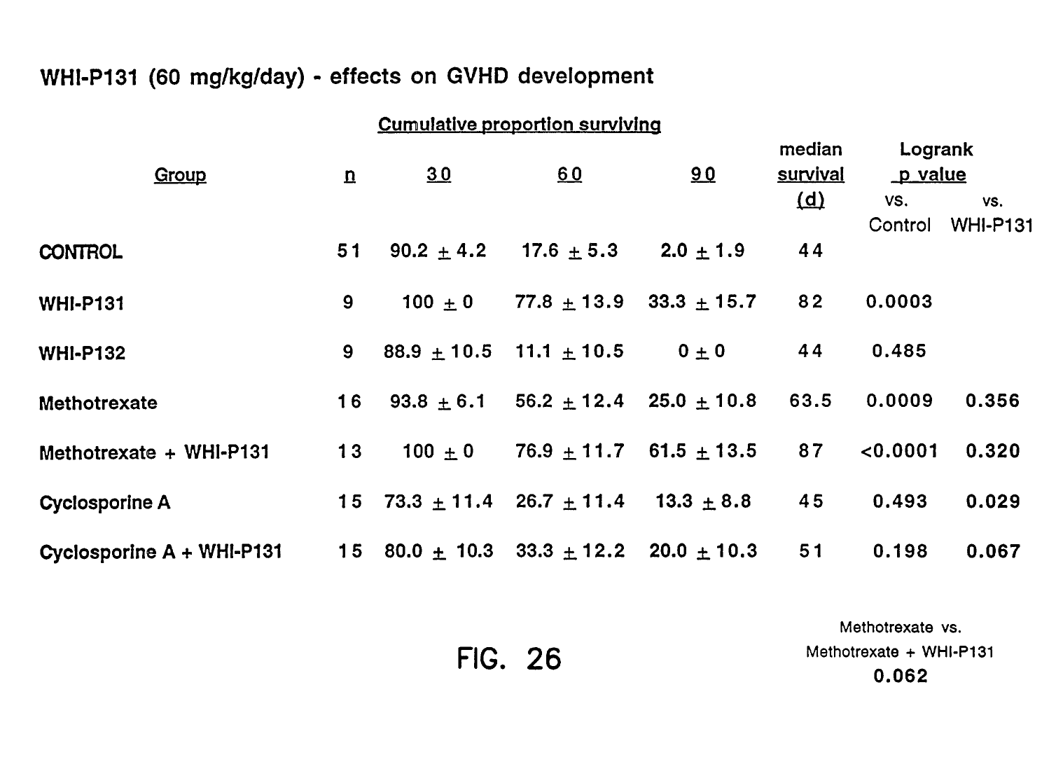

Data from the above assays is presented in FIGS. 21-26.

### Example 7

### Autoimmune Diseases

The ability of a compound to prevent or treat autoimmune diseases (e.g. autoimmune induced diabetes) be determined using assays that are known in the art, or can be determined using assays similar to those described in this example.

C57BL/6 and NOD mice were purchased from Taconic, Germantown, N.Y., and housed under pathogen-free conditions in Animal care facility of Hughes Institute. Jak3^{-/-} (C57BL/6×129/Sv) mice, homologous for disrupted Jak3 gene were generous gift of Dr. J. N. Ihle, St. Jude Children's Research Hospital, Memphis, Tenn. A homozygous Jak3^{-/-} mice were bred to C57BL/6 mice and the offspring of the F1 generation were backcrossed to C57BL/6 mice. After three generations of backcrossing to C57BL/6, the offspring were intercrossed to produce Jak3^{+/+} and wild-type (WT)Jak3^{+/+} mice, which were used in our experiments. Induction of LDSTZ model of diabetes.

C57BL/6 male mice or JAK3-deficient and WT mice (8-10-wk-old) were injected intraperitoneally with low-dose (40 mg/kg) of STZ (Sigma, St Louis, Mo.) daily for 5 consecutive days, for induction of autoimmune experimental diabetes (LDSTZ). STZ was dissolved in citrate buffer pH 4.0 on ice, and injected within 10 min of preparation. Mice were monitored for diabetes development by testing blood glucose from the second week (day 7) after the STZ administrations by One Touch Profile diabetes tracking system (Lifescan, Milpitas, Ca). Mice with glycemia over 220 mg/dl on three consecutive tests were considered diabetic, with the first detection of chronic glycemia taken as the date of diabetes onset. A group of C57BL/6 males was treated with WHI-P131-100 mg/kg/day, i.p., devided in two equal doses, from a beggining of the experiment till day 25. As WHI-P131 was solubilazed in 10% DMSO in PBS, control mice were treated with 10% DMSO in PBS, using the same conditions as described above.

Treatment of NOD Females with WHI-P131 and Assessment of Diabetes Development.

NOD females were treated from 5- or 10-wk of age with different dose of WHI-P131, daily, i.p. Control mice were treated with 10% DMSO in PBS. Mice were monitored for diabetes development by testing blood glucose from 10 wk of age by One Touch Profile diabetes tracking system, as described above.

Intraperitoneal Glucose Tolerance Test (IPGTT).

Intraperitoneal glucose tolerance test (IPGTT) was performed in the group of non-diabetic WHI-P131-treated and vehicle-treated control NOD females on the end of experimental period (25 wk of age of NOD mice). Mice were fasted for 10 hours, and the glucose solution (1.5 g/kg body weight) was injected i.p. Before and after injection of glucose, blood samples were taken and blood glucose levels were measured (as described above) at 0, 30, 60 and 120 min time points. Histology. The group of control and WHI-P131-treated non-diabetic NOD females was sacrificed at the end of experimental period, at 25 wksof age, and characteristic histopathologic lesion of islets (insulitis) was evaluated in each mouse scoring at least 25 islets per mouse. Briefly, pancreas was removed, fixed in 10% formalin, parafin embedded, cut and stained with hematoxylin and eosin for light microscopic examinations. All islets sampled from three nonoverlapping pancreatic levels were assigned an insulitis score as follows: 0—no visible lesions; 1—peri-insulitis with no islet penetration; 2—<25% of the islet infiltrated; 3—>25% of the islet infiltrated; 4—end stage.

Adoptive Transfer of Diabetic Splenocytes to NOD-Scid/Scid Females and Assesment of Diabetes Development.

Single-cell suspensions of splenic leucocytes pooled from 8-10 diabetic NOD females were prepared by passage through Nitex 110 mesh, and red blood cells were lysed in 10×Gey's solution. Aliquots of 1×10⁷ splenocytes were adoptively transferred intravenously into 4-week-old NOD/Lt-scid/scid females (The Jackson Laboratory, Bar Harbor, Me.). WHI-P131 treatment (50 mg/kg) or control treatment (10% DMSO in PBS) of NOD-scid mice started at the same time. Mice were monitored for diabetes development by testing urinary glucose every week from second week after the transfer by Chemstrip uGK strips (Boehringer Mannheim, Indianapolis, Ind.). Mice with glycosuria over 500 mg/dl (+++) on consecutive weekly tests were considered diabetic, with the first detection of chronic glycosuria taken as the date of IDDM onset.

Statistical Analysis.

Statistical analysis was done by using unpaired Student's t-test (IPGTT and insulitis data) and ANOVA test (differences in glycemic level between the experimental groups). Experimental differences in IDDM incidence studies in WHI-P131-treated and control NOD and LDSTZ-treated mice or in adoptively transferred scid mice were assessed by the Kaplan-Meier life table analysis using Mantel-Cox test. The p value <0.05 was considered as statistically significant.

Development of LDSTZ Diabetes is Inhibited in JAK3-Deficient Mice

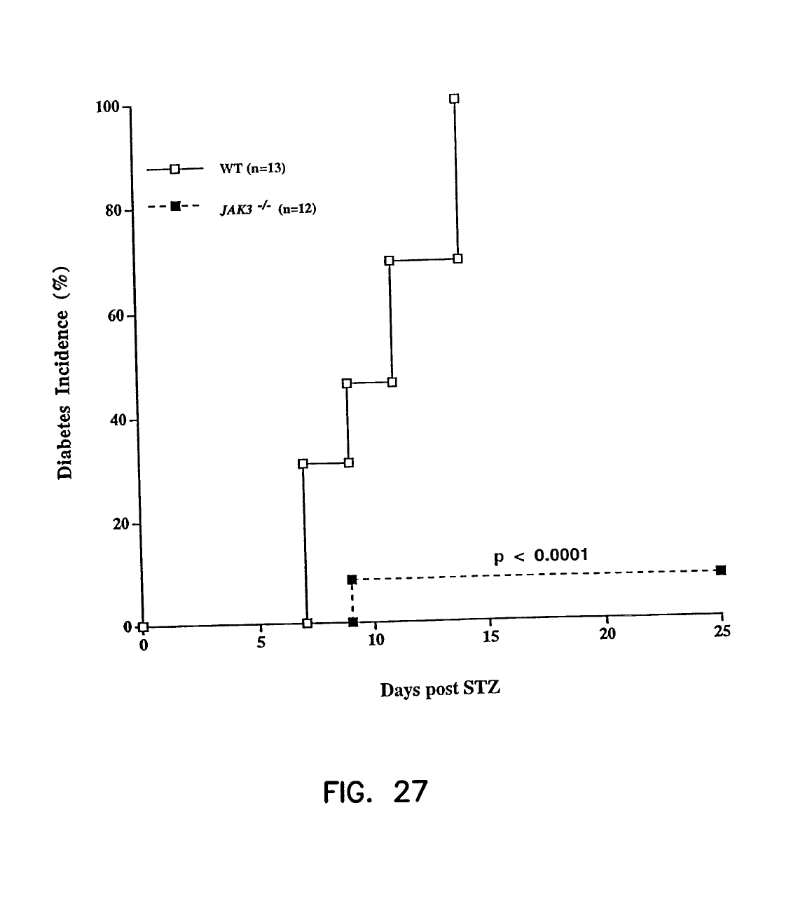

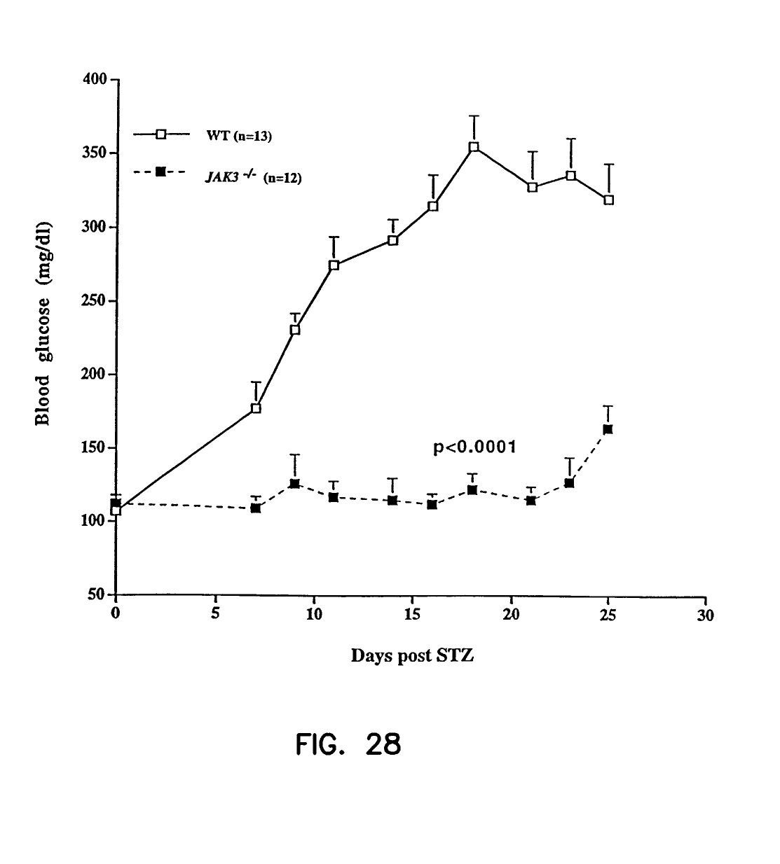

FIG. 27 shows cummulative diabetes incidence (A) and blood glucose level (B) in JAK3-deficient and WT mice treated by low-dose STZ. While 13/13 (100%) of WT mice developed hyperglycemia till day 14 post first STZ injection, only 1/12 (8.3%) of JAK3-deficient mice became diabetic in entire experimental period of 25 days (p<0.0001) (FIG. 27). WT mice exhibited increase of blood glucose from day 7, reaching the hyperglycemic level (>220 mg/dl) on day 9 post first STZ injection (FIG. 28). In contrary, JAK3-deficient mice stayed normoglycemic throught entire experimental period (p<0.0001 compared to WT glycemic level by ANOVA) (FIG. 28).

Inhibition of Diabetes Development in LDSTZ Model of Disease by JAK3 Kinase Inhibitor WHI-P131.

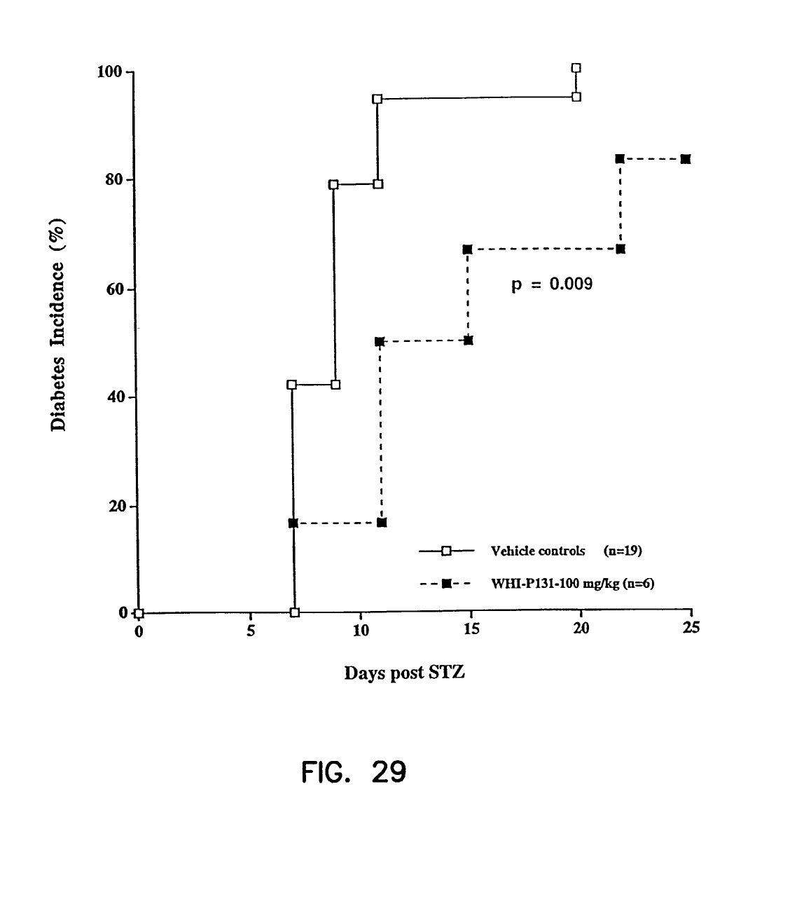

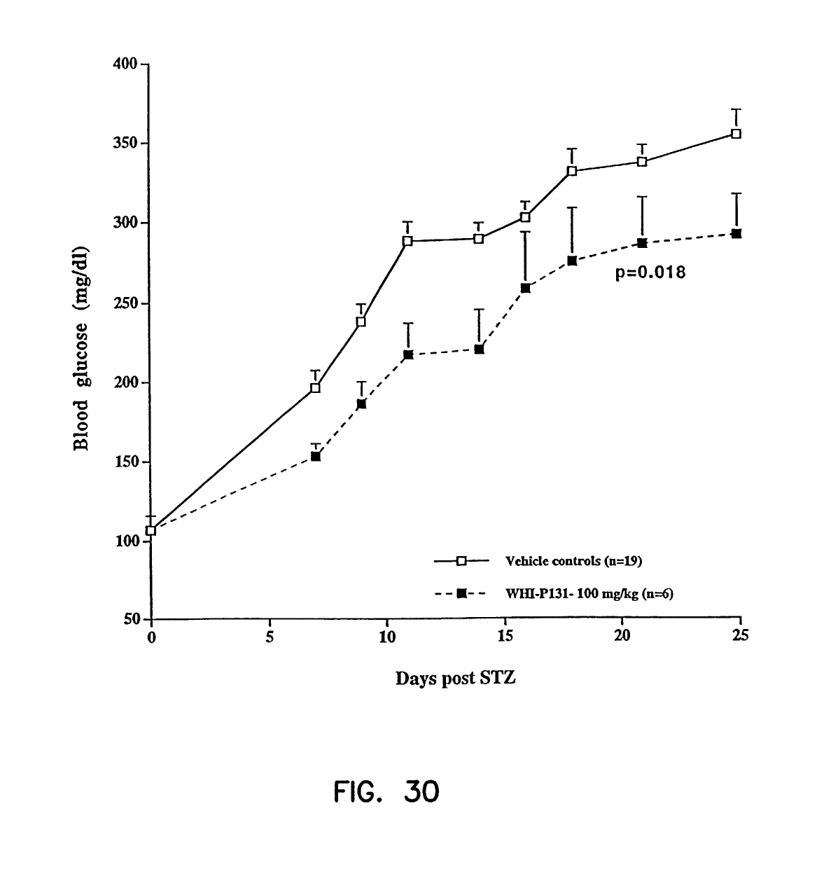

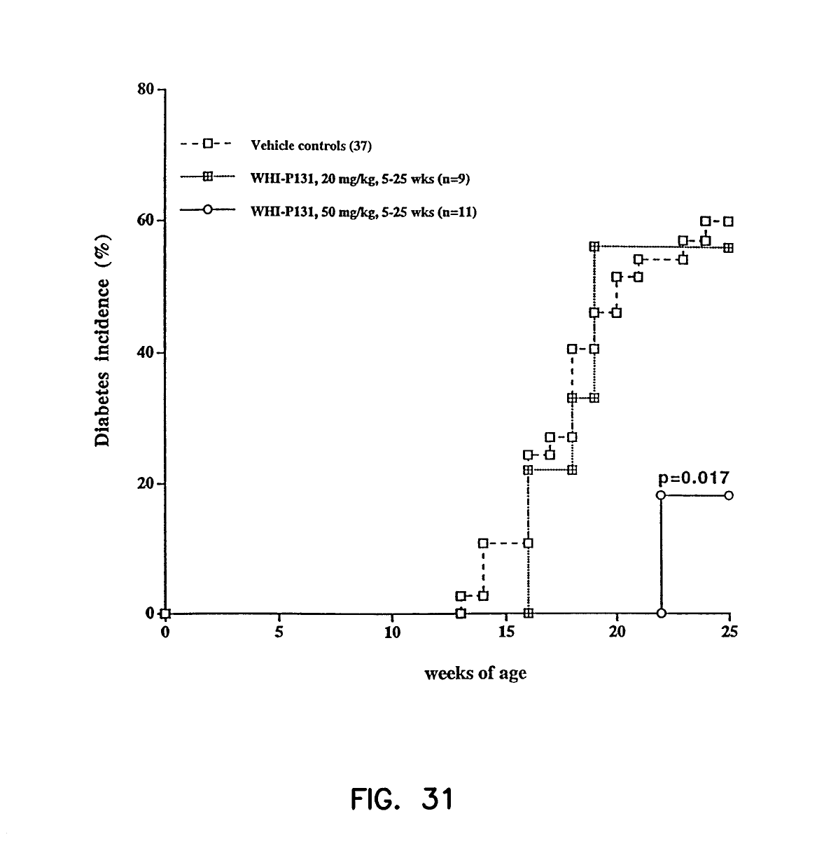

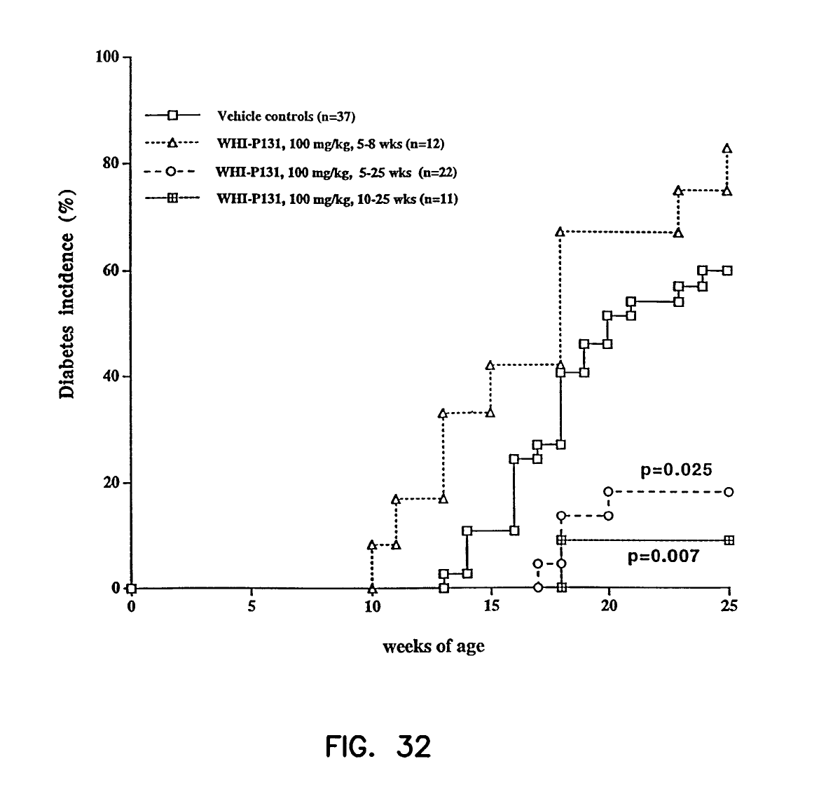

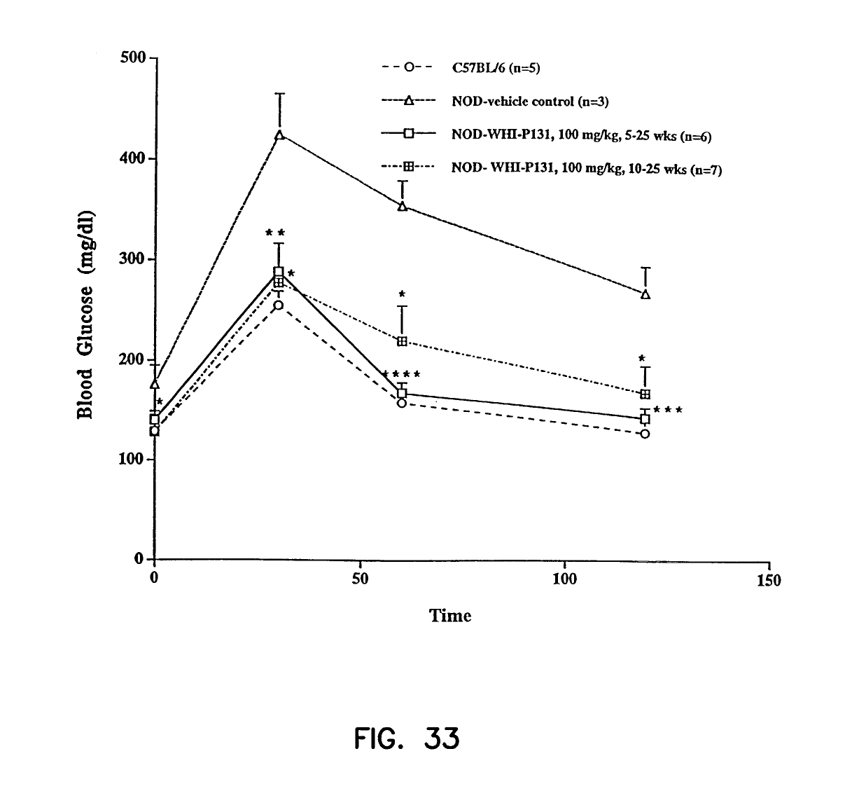

Daily treatment of C57BL/6 males by 100 mg/kg of WHI-P131 (2 doses) i.p., initiated from the first day of STZ injections, inhibited the development of LDSTZ diabetes (FIG. 29). While 20/38 (52.6%) of control mice developed diabetes on day 7, followed by 32/38 (84.2%) of diabetic mice on day 9 and 36/38 (97.4%) of diabetic mice on day 11 post first STZ injection, only 4/20 (20%) WHI-P131-treated mice became diabetic on day 7, followed by 10/20 (50%) on day 9 and 13/20 (65%) on day 11 (FIG. 29). FIG. 30 shows that WHI-P131-treated mice exhibited significantly lower (p=0.027) blood glucose level throught entire experimental period compared to the control mice.

Prevention of IDDM Development in NOD Females by WHI-P131 Treatment.

The diabetes incidence in NOD females treated daily with: a) 20 and 50 mg/kg of WHI-P131 from 5 to 25 wks of age (FIG. 27), and b) 100 mg/kg of WHI-P131 from 5 to 8, 5 to 25 and 10-25 wks of age (FIG. 28) was studied. While diabetes appeared at 13 wks of age in DMSO-treated mice (control), the WHI-P131—(50 mg/kg) treated mice started to develop diabetes at 22 wks of age (FIG. 27). At 25 wks, 22/37 (60%) of control mice became diabetic, while only 2/11 (18%) of NOD females treated with 50 mg/kg of WHI-P131 developed diabetes (p=0.017). Daily treatment with 20 mg/kg of WHI-P131 did not show protective effect on diabetes development—5/9 (56%) of treated mice developed diabetes till 18 wks of age (FIG. 27). We asked whether short course of treatment with 100 mg/kg of WHI-P131, from wks 5 to 9 of life, could have a lasting protective effect. FIG. 28 shows that such treatment did not result with the protection from diabetes development—diabetes started at 10 wks and by 25 wks of age 10/12 (83%) of NOD females became diabetic. Then, it was studied whether later beginning of treatment (at 1 Owks of age) with 100 mg/kg of WHI-P131 could be effective in the prevention of diabetes. FIG. 28 shows that treatment with 100 mg/kg of WHI-P131, initiated at 10 wks of age, is as effective as treatment initiated earlier, at 5 wks of age—diabetes incidence reached 9% (1/11) at 25 wks of age under the first treatment (p=0.007 compared to controls) and 18% (4/22) under the later one (p=0.025 compared to controls).

Group of normoglycemic NOD females treated with DMSO (n=3) or with 100 mg/kg of WHI-P131 for 20 (n=6) or 15 weeks (n=7) was fasted on the end of experimental period (at 25 wks of age) and IPGTT was performed. Non-diabetic, non-treated C57BL/6 mice (n=5) were used as controls in IPGTT test, as well. The results—blood glucose levels were similar between the C57BL/6 mice and both groups of WHI-P131-treated NOD mice during 120 min time period post glucose challenge. In contrast, normoglycemic DMSO-treated mice exhibited a significantly higher blood glucose level at each observed time point then either of WHI-P131-treated groups.

Further, histological examination of the insulitis level of the pancreata obtained from the mice analyzed in IPGTT was done. Insulitis score of controls (n=3) was 1.43±0.15, while insulitis score of NOD females treated by WHI-P131 from 5-25 wks of age was significantly (p=0.026) lower—0.86±0.12. However, insulitis score of NOD females treated by WHI-P131 from 10-25 wks of age was not different from controls (1.42±0.24).

Prevention of Diabetes Development in Adoptively Transferred NOD-Scid/Scid Females by WHI-P131 Treatment.

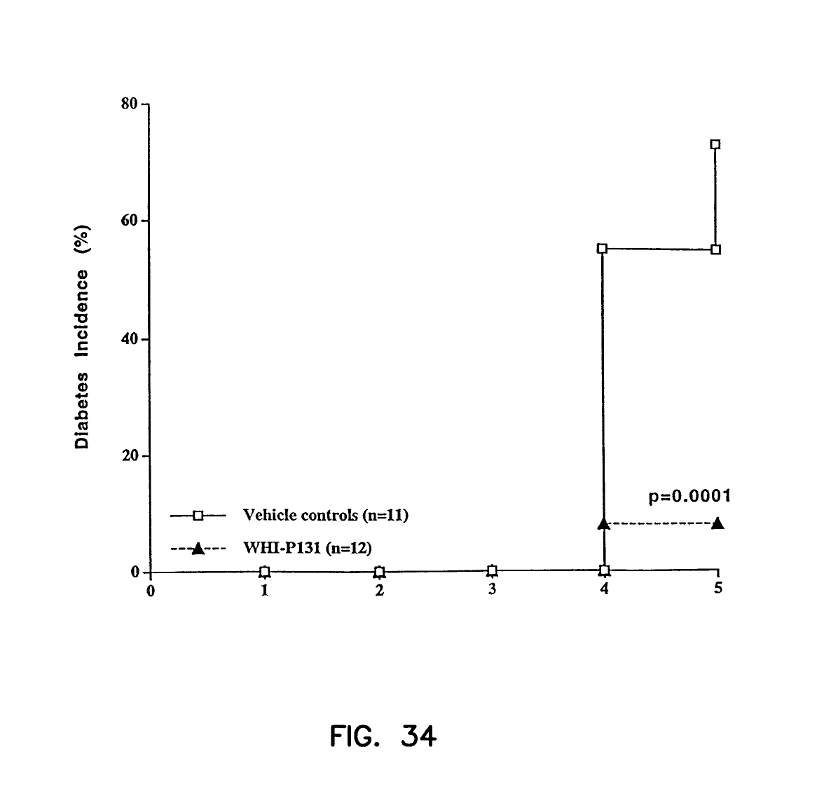

As WHI-P131 showed capability to prevent diabetes development in NOD females during the prediabetic phase of spontaneous development of type I diabetes, next we wanted to determine whether WHI-P131 was capable of suppressing effectors from already diabetic mice in transferring disease to NOD-scid mice. Two groups of NOD-scid females were adoptively transferred with 1×10⁷ splenocytes from diabetic NOD females and one group was treated daily from the day of transfer with 50 mg/kg of WHI-P131 (n=12), while another one was treated by 10% DMSO (n=11) (FIG. 34). While 6/11 (55%) of control NOD-scid became diabetic at 4 wk, followed by 8/11 (73%) of diabetic mice at week 5 post adoptive transfer, only 1/12 (8%) of WHI-P131-treated NOD-scid females became diabetic in the same experimental period (FIG. 4). Clearly, diabetes development post adoptive transfer was significantly (p<0.002) prevented by WHI-P131 treatment.

### Example 8

### Prolongation of Allograft Survival Without Impairment of Islet Cell Function

The ability of a compound to prolong allograft survival can be determined using assays that are known in the art, or can be determined using assays similar to those described in this example.

Male C57BL/6 mice (H-2^{b}) 8-12-wk-old were used as recipients and BALB/c (H-2^{d}) males of the same age were used as a donors. Both strain of mice were purchased at Taconic, Germantown, N.Y., and housed under pathogen-free conditions in Animal care facility of Hughes Institute. Jak3^{-/-} (C57BL/6×129/Sv, H-2^{b}) mice, homologous for disrupted Jak3 gene were generous gift of Dr. J. N. Ihle, St. Jude Children's Research Hospital, Memphis, Tenn. A homozygous Jak3^{-/-} mice were bred to C57BL/6 mice and the offspring of the F1 generation were backcrossed to C57BL/6 mice. After three generations of backcrossing to C57BL/6, the offspring were intercrossed to produce Jak3^{-/-} and wild-type (WT)Jak3^{+/+} mice. 10-12-wk-old Jak3^{-/-} and WT males were used as recipients of BALB/c islets.

Mixed Lymphocyte Reaction (MLR).

For MLR assay, responder cells (splenocytes obtained from 10-wk-old C57BL/6 males) were plated in triplicates in 96-well-plates in the concentration of 4×10⁵/100 μl of RPMI (Life Technologies, Grand Island, N.Y.) with addition of 10% fetal bovine serum (Laboratories Inc., Logan, Utah). Mitomycin-treated stimulators (splenocytes obtained from 10-12-wk-old BALB/c males) were added in the concentration of 8×10⁴ in 50 μl. WHI-P131 was added in different concentrations to a final volume of 200 μl and cells were cultured in 5% CO2 with humidified air in an incubator at 37° C. for 5 days. Then, a colorimetric assay for the quantification of cell proliferation, based on the cleavage of the tetrazolium salt WST-1 by mitochondrial dehydrogenases in viable cells (Boehringer Mannheim, Indianapolis, Ind.), was performed following manufacturer's instructions. The absorbance was measured at 450/690 nm on a Multiskan MS microplate scanner. The value of cell proliferation was obtained by diminishing O.D. value of proliferation of stimulated cells by O.D. value of non-stimulated cells (O.D. values of non-stimulating cells were between 0.370-0.450).

Apoptosis Detection.

C57BL6 splenocytes (3×10⁶/ml) were cultured in 24-well plate for 20 h in 500 μl of RPMI—1640 under the conditions described above. WHI-P131 was added in the concentrations of 0.1, 1, 10 and 100 μg/ml. Apoptotic cell death was detected by TUNEL, using InSitu Cell Detection Kit, Fluorescein (Boehringer Mannheim, Indianapolis, Ind.). After the culture period, cells were washed, fixed, permeabilised and stained following manufacturer's instructions and apoptosis was analyzed by flow cytometry, using FACS Calibur (Becton Dickinson, San Jose, Calif.).

Flow Cytometric (FACS) Analysis.

Single cell suspension of splenocytes was prepared from WHI-P131—(130 mg/kg/day) or vehicle-treated C57BL/6 mice, red blood cells were lysed by lysis buffer and splenocytes (1×10⁶) were stained with 1:100 dilution of following anti-mouse monoclonal antibodies (Ab): anti-CD3-FITC Ab (clone 145-2C11), anti-CD4-FITC Ab (clone GK1.5), anti-CD8-PE Ab (clone 53-6.7) and anti-CD19-PE Ab (clone 1D3). All Abs were purchased from Pharmingen, San Diego, Calif. Stained splenocytes were analyzed by FACS Calibur, as described above.

Islet Isolation.

Islets of Langerhans were isolated from BALB/c males by the bile duct perfusion with 3-4 ml of collagenase P (3 mg/ml) (Boehringer Mannheim, Indianapolis, Ind.) and deoxyribonuclease (0.1 mg/ml) (Sigma, St. Louis, Mo.), as described previously Cetkovic-Cvrlje M, et al., 1997, Diabetes, 46: 1975-1982). Islets were hand-picked 3-4 times under the dissecting scope before islets were free of exocrine tissue, vessels, lymph nodes and ducts and ready for transplantation.

Allogeneic Islet Transplantation and Drug Treatment.

Four hundred islets were placed in Hamilton syringe and transplanted under the left kidney capsule of each diabetic recipient mouse. Recipients were rendered diabetic with a single i.p. dose of streptozotocin (200 mg/kg; Sigma, St. Louis, Mo.) 1 wk before transplantation. Blood glucose level was measured by One Touch Profile glucose monitor system (Lifescan, Milpitas, Ca). Only diabetic mice with glycemia over 350 mg/dl were used as transplant recipients. Allograft function was monitored by serial blood glucose measurements. Primary graft function was defined as a blood glucose under 200 mg/dl on day 3 post transplantation and graft rejection was defined as a rise in blood glucose exceeding 250 mg/dl on two consecutive measurements, following a period of primary graft function. Recipients were treated daily with high-dose (20 mg/kg) of cyclosporine A (Sigma, St. Louis, Mo.), WHI-P131 (50 and 75 mg/kg, divided in three doses), WHI-P132 (50 mg/kg, divided in three doses) or with vehicle control from the day of transplantation until the day of rejection. All injections were given i.p.

Intraperitoneal Glucose Tolerance Test (IPGTT) in Syngeneic Islet Transplant Recipients.

IPGTT was performed in C57BL/6 recipients that were transplanted with syngeneic islet grafts (400 C57BL/6 islets) and treated with WHI-P131 or vehicle control for two months. Mice were fasted for 10 hours, and the glucose solution (1.5 g/kg body weight) was injected i.p. Before and after injection of glucose, blood samples were taken and blood glucose levels were measured (as described above) at 0, 30, 60 and 120 min time points.

Histopathological Studies.

The vehicle control—(n=3) and WHI-P131-treated C57BL/6 recipients (n=5) of islet allograft were sacrificed on day 14 post transplantation; Jak3^{-/-} recipients (n=6) were sacrificed on day 100 post transplantation; the C57BL/6 recipients of syngeneic islets—vehicle control—(n=4) and WHI-P131-treated (n=5) were sacrificed on day 180 post transplantation. Kidneys bearing grafts were removed, fixed in 10% formalin and embedded in paraffin. Serial sections of graft area were cut and stained with hematoxylin and eosin. For insulin staining, sections were stained by immunoperoxidase method using 1:100 dilution of polyclonal guinea pig anti-insulin antibody (Gpx Insulin, Dako, Carpinteria, Calif.) and 1:100 dilution of secondary antibody conjugated to horse radish peroxidase (Guinea Pig Immunoglobulins HRP, Dako, Carpinteria, Calif.). Sections were briefly counterstained with hematoxylin and mounted for light microscopic examination.

Statistical Analysis.

Statistical analysis was done by using unpaired Student's t-test (MLR and FACS data). Experimental differences in allograft rejections between the drug-treated and control groups were assessed by the Kaplan-Meier life table analysis using Mantel-Cox test. The p value <0.05 was considered as statistically significant.

Jak3^{-/-} Mice do not Reject Islet Alograft.

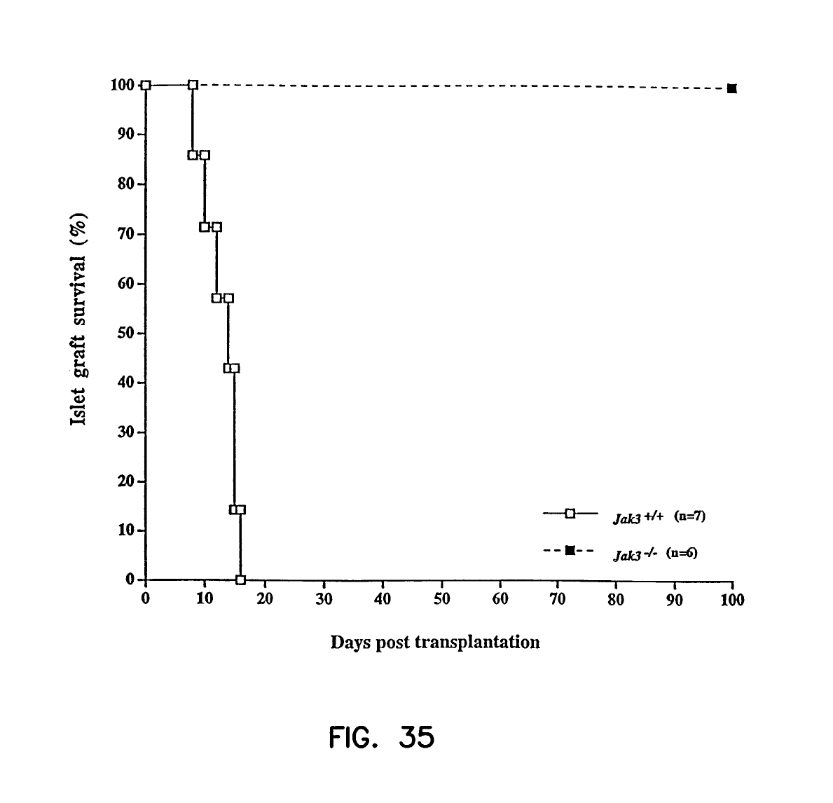

JAK3-deficient males (n=6) and their WT littermates (n=7), rendered diabetic by STZ, were transplanted with BALB/c islets under the kidney capsule and blood glucose level was followed for 100 days post transplantation. While islet allografts of WT controls were rejected with a MST of 12.9±1.1 days, all islet allografts of Jak3^{-/-} recipients survived 100 days post transplantation (FIG. 35). Histological analysis of grafts showed no infiltration (data not shown) to slight mononuclear infiltration of graft area with completely preserved islet morphology.

WHI-P131-Induced Inhibition of MLR Response.

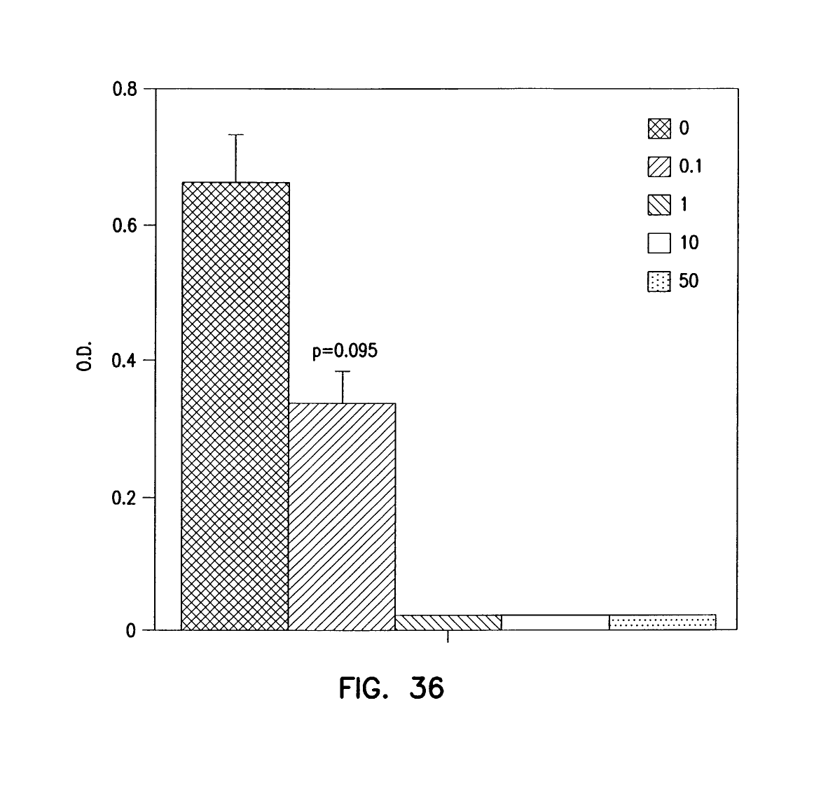

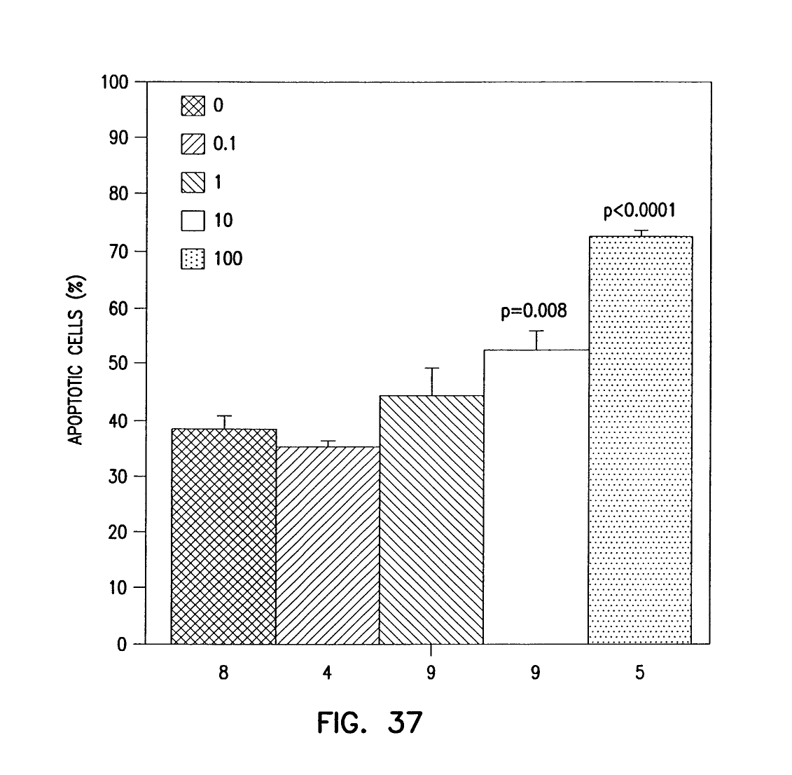

WHI-P131, added in the concentration of 0.1, 1, 10 and 50 μg/ml, inhibited proliferation of alloreactive splenocytes in MLR in a dose-dependent manner (FIG. 36). While the concentration of 0.1 □Fg/ml of WHI-P131 induced statistically significant (p=0.0095) reduction of MLR response, complete abrogation of the response (obtained O.D. values were below the O.D. level of non-stimulating controls) was obtained with addition of 1 μg/ml of WHI-P1131 (FIG. 36). Next we tested whether WHI-P131-induced lymphocyte death was the reason for inhibited MLR response. Therefore, apoptotic splenocytes (TUNEL-positive) were determined after the culture period of 20 h with addition of different concentrations of WHI-P131. FIG. 37 shows that apoptotic cell death of splenocytes cultured with addition of 0.1 and 1 μg/ml of WHI-P131 does not differ from control cells, while addition of 10 and 100 μg/ml significantly increased (p=0.008 and p<0.0001, respectively) apoptotic cell death in comparison to control cell death during the observed culture period. Therefore, WHI-P131 effects on MLR suppression, obtained with lower concentration of the drug (0.1 and 1 μg/ml), seems to not be caused by the induction of cell death.

WHI-P131 Treatment of Recipients Prolonged Islet Allograft Survival.

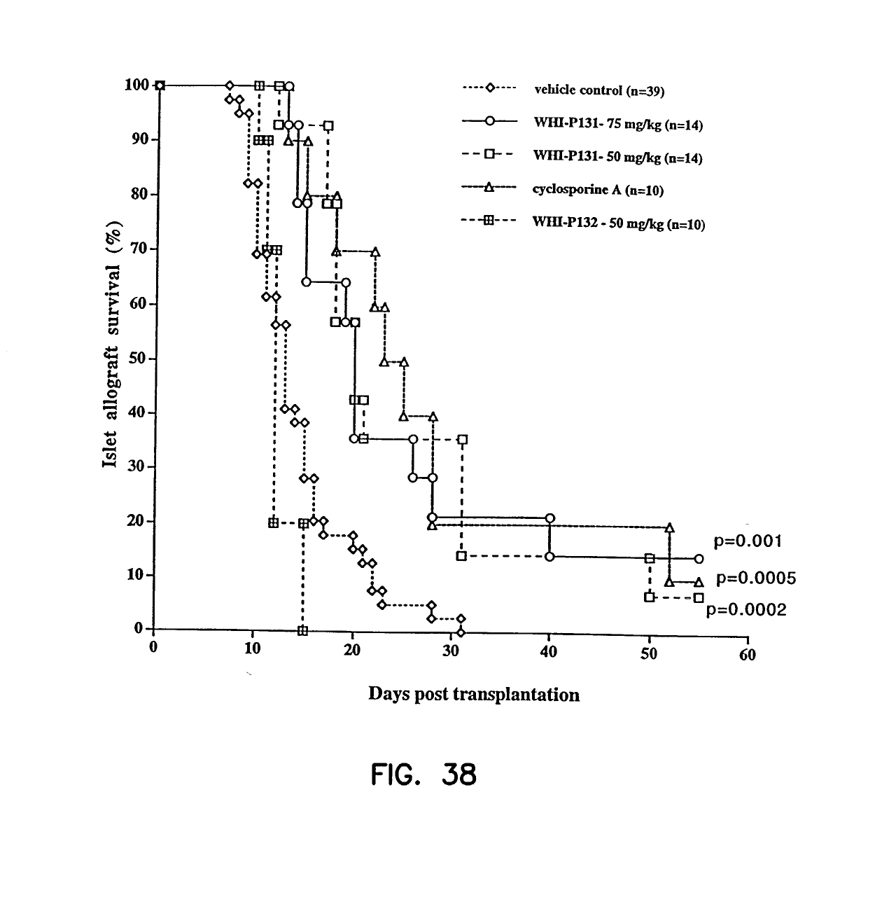

The control C57BL/6 mice (n=39) rejected BALB/c islet allograft with a mean survival time (MST) of 14.1±0.9 days. Daily treatment of recipients (n=10) with high-dose—20 mg/kg—of CsA (18) significantly increased (p=0.0005) allograft survival (MST=27.9±4.6 days) (FIG. 38). Treatments with 50 mg/kg (n=14) or 75 mg/kg (n=14) of WHI-P131 were as effective as CsA treatment in prolongation of allograft survival (MST=24.7±3.4 and 25.3±3.8, respectively) in comparison to controls (p=0.0002 and 0.001, respectively) (FIG. 38).

Histologic examination of the islet allografts that were harvested at 14 days post transplantation from normoglycemic recipients treated with vehicle control (n=3) showed lymphocytic invasion and massive islet destruction, clearly seen with immunostaining for insulin. This finding is in striking contrast with allografts from recipients treated with 50 mg/kg of WHI-P131 (n=5), in which lymphocytic infiltration was present but without extensive destruction of islets. The hyperglycemic vehicle-treated allografts (n=4), harvested at the same time point, were completely invaded by lymphocytes with no remaining insulin-producing cells.

The next experiment was performed with an aim to study the effects of WHI-P131 treatment on splenocyte populations in vivo. C57BL/6 males were treated with 130 mg/kg of WHI-P131 (n=7) and with vehicle control (n=5) for 10 days. Total number of splenocytes was 154±8.5×10⁶ in vehicle-treated controls, while reduced number of splenocytes—119±9.4×10⁶ was found in WHI-P131-treated mice (p=0.025). There were no differences in the percentages of CD3+, CD4+ and CD8+ T-cells and CD 19+ B-cells between the WHI-P131- and vehicle-treated mice (data not shown). However, FIG. 6 shows that slight differences in the number of studied cell populations between the WHI-P131-treated and control groups were found. Thus, the number of CD3+ splenocytes was reduced to 37.7±3.2×10⁶ (however, not statistically significant, p=0.0617) in WHI-P131-treated mice in comparison to 46.9±2.6×10⁶ in controls. The number of CD4⁺ T-cells −16.8±1.2×10⁶ (p=0.0416), as well as B-cells—57.1±5.3×10⁶ (p=0.0267) were reduced in WHI-P131-treated mice compared to the controls—20.5±0.6×10⁶ and 78.2±6.1×10⁶, respectively (FIG. 39).

Function of Transplanted Syngeneic Islets is Preserved after the Long-Term Treatment with WHI-P131.

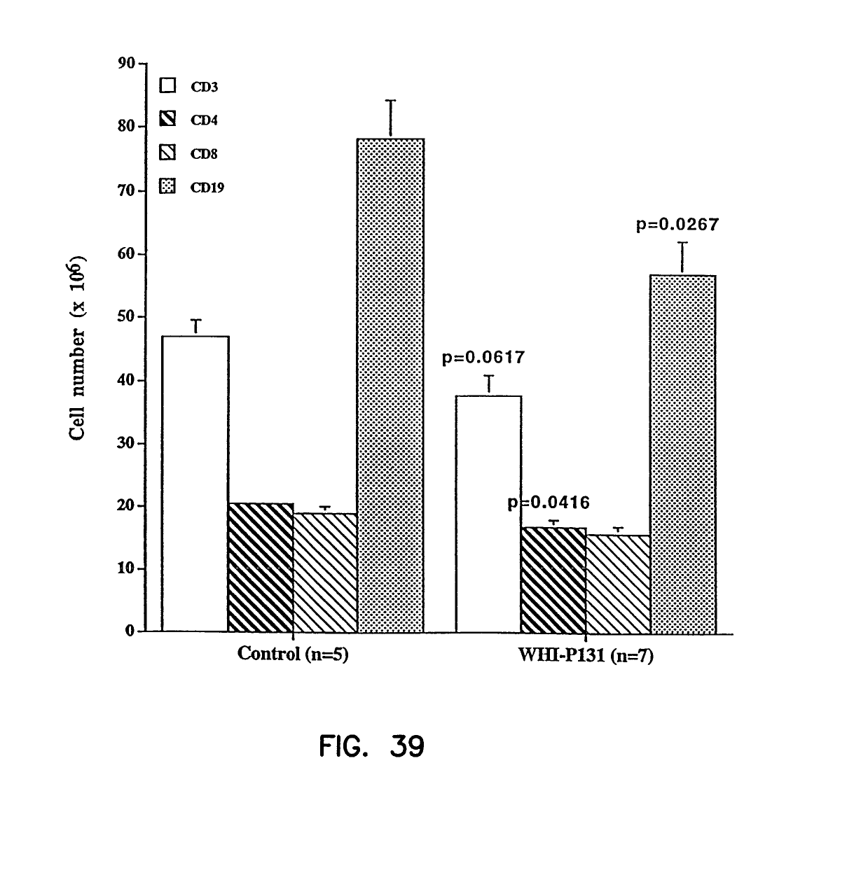

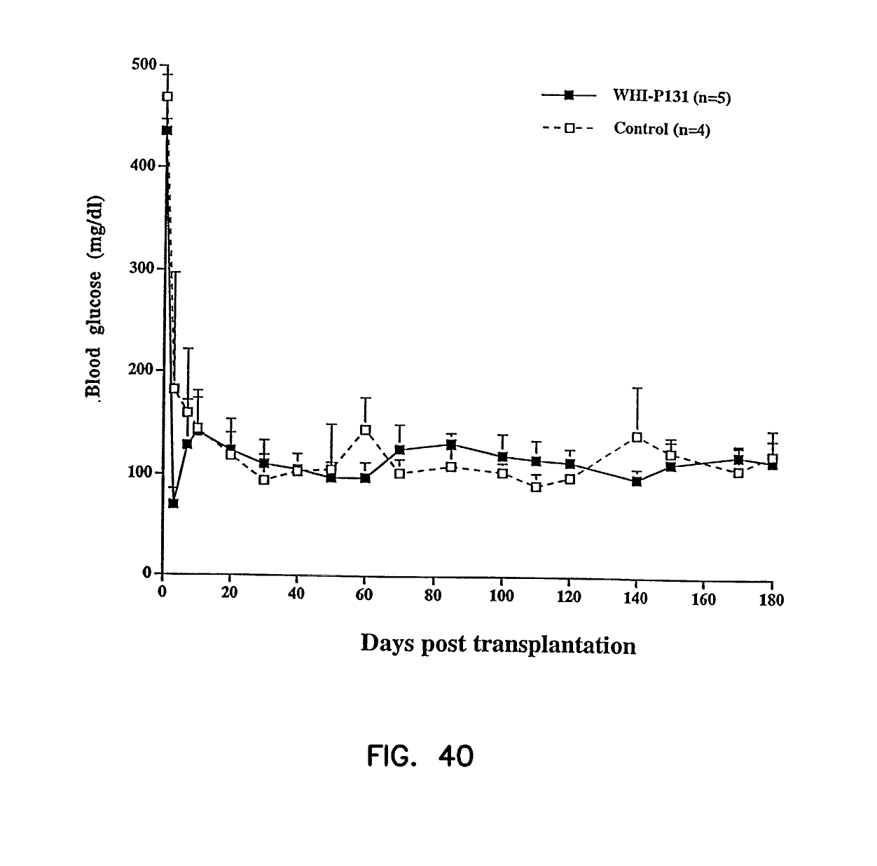

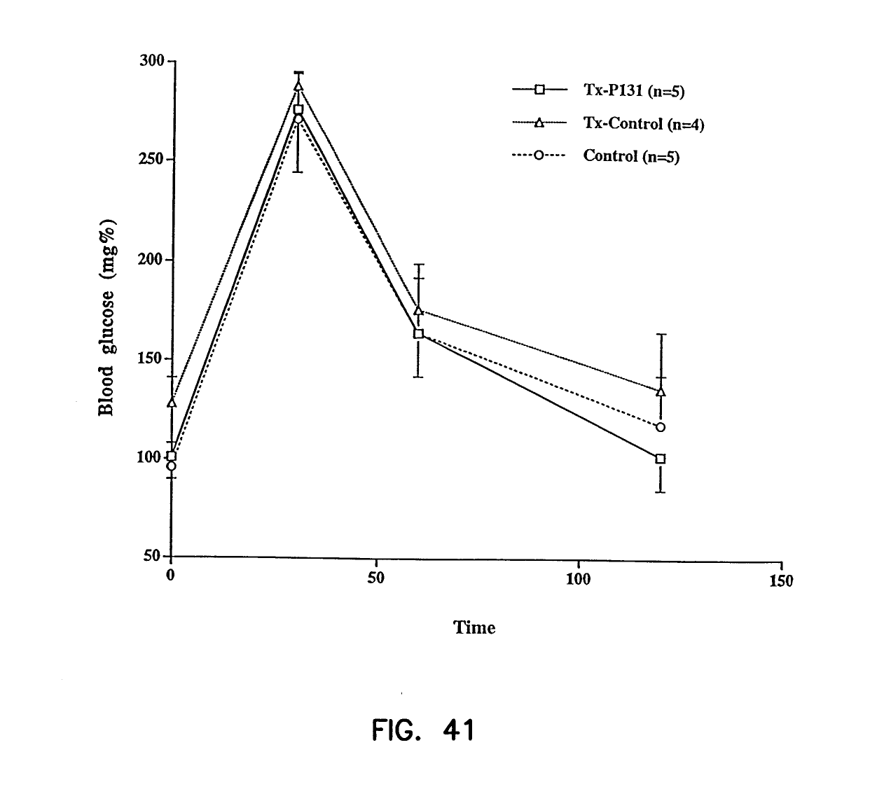

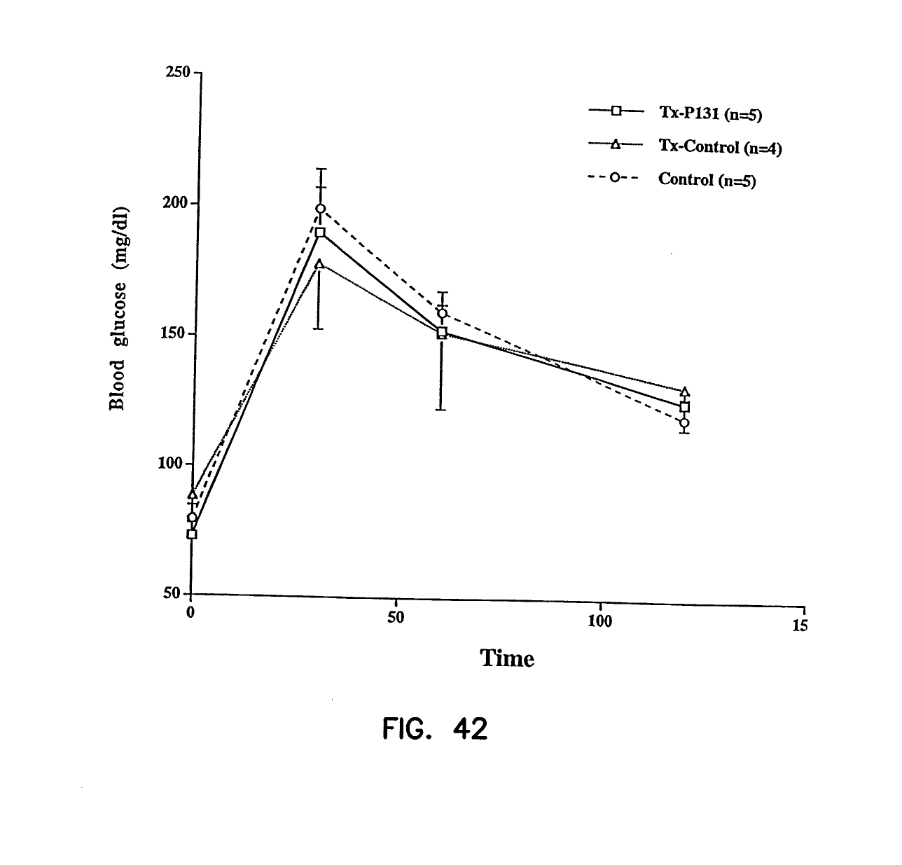

This study was performed to test the effect of long-term treatment (180 days) with 50 mg/kg of WHI-P1131 on syngeneic islet graft function. As it could be seen on FIG. 40, non-fasting blood glucose level did not differ during the entire experimental period of 180 days post transplantation between the vehicle control-(n=4) and WHI-P131-treated (n=5) syngeneic recipients. IPGTT was performed on day 70 of treatment. The nonfasting blood glucose of the controls and P 131-treated recipients at that time was 102.0±6.7 and 124.6±10.8 mg/dl, respectively. IPGTT test showed that there was no significant difference in islet function of non-transplanted, non-treated C57BL/6 males, transplanted vehicle-treated recipients (controls) and transplanted WHI-P131-treated recipients (FIG. 41). Another IPGTT test was performed on the end of experimental period (day 180 post transplantation). The non-fasting blood glucose level at that time was 120.9±12.5 in vehicle control- and 115.2±9.2 mg/dl in WHI-P131-treated recipients. IPGTT test showed again that islet function of WHI-P131-treated recipients was not different from islet function of either control recipients or non-transplanted C57BL/6 mice.

Representative examples from the light microscopical evaluation of the vehicle control- and WHI-P131-treated syngeneic islet grafts are illustrated in FIG. 8. Hematoxylin and eosin staining of vehicle control and WHI-P131-treated grafts showed that there were no significant morphological changes between them. Immunohistochemical analysis confirmed that insulin expression in WHI-P131-treated grafts was comparable to that of the grafts of the vehicle control-treated recipients.

### Example 9

### C57BL/6 and BALB/c Mice.

8-10-wk-old C57BL/6 (H-2^{b}) male mice and 6-8-wk-old BALB/c (H-2^{d}) male mice were purchased from Taconic, Germantown, N.Y. Mice were housed in a controlled environment (12-h light/12-h dark photoperiod, 22±1° C., 60±10% relative humidity), which is fully accredited by the USDA (United States Department of Agriculture). All husbandry and experimental contact made with the mice maintained specific pathogen free (SPF) conditions. All mice were kept in Micro-Isolator cages (Lab Products, Inc., Maywood, N.Y.) containing autoclaved food (Harlan Teklad LM-485), water and bedding. Animal studies were approved by the Parker Hughes Institute Animal Care and Use Committee, and all animal care procedures conformed to the Principles of Laboratory Animal Care (NIH publication #85-23, revised 1985).

Mitogen Stimulation Assays.

Splenocytes (4×10⁶/ml RPMI 1640 medium, supplemented by 10% fetal calf serum) from 9-wk-old C57BL/6 males were used as responders in phytohemagglutinin (PHA)—or concanavalin A (Con A)-induced T-cell stimulation/proliferation assays. (Cetkovic-Cvrlje M, Gerling I C, Muir A, Atkinson M A, Elliott J F, Leiter E H. Retardation or acceleration of diabetes in NOD/Lt mice mediated by intrathymic administration of candidate beta cell antigens. Diabetes. 1997; 46:1975-1982). Cells (2×10⁵/200 μL final volume/sample) in triplicate wells of 96-well microplates were stimulated with PHA in the presence or absence of WHI-P131. PHA (Sigma, St. Louis, Mo.) was used at a final concentration of 5 μg/ml. Con A (Sigma, St. Louis, Mo.) was used at a final concentration of 2 μg/mL. WHI-P131 was used at the final concentrations of 0.1, 1, 10 and 50 μg/ml. Controls were treated with vehicle alone without any added WHI-P131. Cells were cultured at 37° C. for 3 days in a humidified 5% CO₂ atmosphere and their mitogenic response was examined using a colorimetric assay for the quantification of cell proliferation, based on the cleavage of the tetrazolium salt WST-1 (Boehringer Mannheim, Indianapolis, Ind.) by mitochondrial dehydrogenases in viable cells. The absorbance at 450/690 nm was measured using a Multiskan MS microplate scanner. The O.D. values of the non-stimulated samples were used for comparison as baseline controls.

Mixed Lymphocyte Reaction (MLR).

We used a one-way MLR assay to measure the in vitro responses of mouse splenocytes to alloantigen. Single-cell suspensions of splenocytes obtained from 9-wk-old C57BL/6 males were used as responder cells and mitomycin-treated splenocytes obtained from 10-12-wk-old BALB/c males were used as stimulators. Responders were plated in triplicate in 96-well-plates (4×10⁵/100 μl/sample). Stimulators (8×10⁴ in 50 μl) were added to each well. WHI-P131 (50 μL) was added to the wells to yield final concentrations of 0.1, 1, 10 and 50 μg/ml. Controls were treated with vehicle alone without any added WHI-P131. Cells were cultured for 5 days and a colorimetric WST-1 assay was performed, as described above for the mitogen response studies.

Pre-Transplant Total Body Irradiation (TBI).

For pre-transplant conditioning, recipient C57BL/6 and BALB/c mice, positioned in a pie-shaped Lucite holder (Braintree Scientific Inc., Boston, Mass.) underwent TBI (7.5 Gy and 6.0 Gy, respectively) one day prior to bone marrow transplantation, which was delivered by a Cesium Instrument (JL Sheppard Labs, 47.08 rad/min). Recipients were given antibiotic-supplemented water (sulfamethoxazole/trimethoprim, Hi-Tech Pharmacal, Amityville, N.Y.) starting the day before transplantation.

Bone Marrow Transplantation (BMT).

Donor bone marrow (BM) was collected into RPMI 1640 medium supplemented with L-glutamine (Cellgro) (Mediatech, Hendon, Va.) by flushing the shafts of femurs and tibias and cell suspensions were prepared as previously described. (Hanson MS, Cetkovic-Cvrlje M, Ramiya VK, et al. Quantitative thresholds of MHC class II I-E expressed on hemopoietically derived antigen-presenting cells in trans genic NOD/Lt mice determine level of diabetes resistance and indicate mechanism of protection. J Immunol. 1996; 1576:1279-1287). In parallel, single cell suspensions of donor splenocytes (S) were prepared from minced spleens as a source of GVHD-causing T-cells. The cells were washed and resuspended for i.v. injection via the caudal vein. The standard BM/S inoculum consisted of 25×10⁶ BM cells and 25×10⁶ splenocytes in 0.5 ml of RPMI 1640.

Graft-Versus-Host Disease (GVHD) Monitoring.

BMT recipients were monitored daily for any clinical evidence of GVHD (weight loss, manifestations of skin erythema, allopecia, hunching, diarrhea) and survival during the 85-day observation period. Survival times were measured from the day of BMT (day 0).

Evaluation of Engraftment Status After BMT.

The allo-engraftment was documented by flow cytometric (FACScan, Becton Dickinson, Mountain View, Calif.) H-2Dd typing of peripheral blood nucleated cells using FITC-labeled anti-H-2Dd antibody (clone 34-2-12, Pharmingen, San Diego, Calif.) which marks BALB/c cells. Immunofluorescent staining of cells and flow cytometry were performed using standard procedures. (Uckun F M, Ledbetter J A. Immunobiologic differences between normal and leukemic human B-cell precursors. Proc. Natl. Acad Sci USA. 1988; 85:8603-8607; and Uckun F M, Myers D E, Jaszcz W, Haissig S, Gajl-Peczalska K, Ledbetter J A. Temporal association of CD40 antigen expression with discrete stages of human B-cell ontogeny and the efficacy of anti-CD40 immunotoxins against clonogenic B-lineage acute lymphoblastic leukemia as well as B-lineage non-Hodgkin's lymphoma cells. Blood. 1990; 76:2449-2456).

Drug Treatments.

For GVHD prophylaxis—injections of WHI-P131, WHI-P132, Methotrexate (MTX) (Immunex Corporation, Seattle, Wash.) or vehicle control were administered to recipient mice. The JAK3-inhibitory dimethoxyquinazoline compound WHI-P131 [4-(4′-hydroxyphenyl)-amino-6,7-dimethoxyquinazoline] (Sudbeck E A, Liu X-P, Narla R K, et al. Structure-based design of specific inhibitors of Janus kinase 3 as apoptosis-inducing anti-leukemic agents. Clin. Cancer Res. 1999; 5:1569-1582.) (60 mg/kg/day in 3 divided doses) and the structurally related control dimethoxyquinazoline compound WHI-P132 [4-(2′-hydroxyphenyl)-amino-6,7-dimethoxyquinazoline] (Sudbeck E A, Liu X-P, Narla R K, et al. Structure-based design of specific inhibitors of Janus kinase 3 as apoptosis-inducing anti-leukemic agents. Clin. Cancer Res. 1999; 5:1569-1582.) (50 mg/kg/day day in 3 divided doses), which does not inhibit JAK3, were administered daily starting on day 0. These dimethoxyquinazoline compounds were synthesized and characterized as previously described in detail. (Sudbeck E A, Liu X-P, Narla R K, et al. Structure-based design of specific inhibitors of Janus kinase 3 as apoptosis-inducing anti-leukemic agents. Clin. Cancer Res. 1999; 5:1569-1582.). WHI-P131 and WHI-P132 were administered intraperitoneally (i.p.) in 200 μL PBS supplemented with 10% DMSO as the vehicle, as previously reported. (Uckun F M, Ek O, Liu X-P, Chen C-L. In vivo toxicity and pharmacokinetic features of the Janus kinase 3 inhibitor WHI-P131 [4-(4′-hydroxyphenyl)-amino-6,7-dimethoxyquinazoline]. Clin. Cancer. Res. 1999; 5:2954-2962). In some experiments, treatment with WHI-P131 and WHI-P132 was delayed until day 18. The standard anti-GVHD drug MTX (10 mg/m²/day once daily) was used for comparison. MTX was administered i.p. on days 1, 3, 6 and 11 post BMT.

Statistical Analysis.

Group comparisons of continuous variables were done using Student's t-tests. The survival data were analyzed by life-table methods. P-values of less than 0.05 (log-rank test) were considered significant.

Histopathologic Examination of Tissues.

Recipient mice were electively sacrified at specified time points, necropsied, and tissues were removed and fixed in 10% neutral buffered formalin. Fixed tissues were embedded in paraffin, cut into 4 μm sections and stained with hematoxylin and eosin. All histopathology slides were blindly coded and graded by a certified veterinary pathologist (B.W.). Livers were scored positive for GVHD when there was a periportal infiltrate, lungs were scored positive when there was evidence of vasculitis with a lymphocytic infiltrate, skin was scored positive when there was single cell necrosis, and colon was scored postive when there was single cell necrosis or crypt dropout. After initial scoring, all slides were reviewed for GVHD grading, as described. (Bryson J S, Jennings C D, Caywood B E, Dix A R, Lowery D M, Kaplan A M. Enhanced graft-versus-host disease in older recipient mice following allogeneic bone marrow transplantation. Bone Marrow Transplantation. 1997; 19:721-728.) Our GVHD grading system was as follows: Liver: Grade 0.5, focal portal lymphoid infiltrate; Grade 1, widespread portal lymphoid infiltrate; Grade 2, focal bile duct invasion or cellular injury; Grade 3, multiple foci of bile duct injury and regeneration; Grade 4, widespread injury and destruction of bile ducts; Small and large intestine: Grade 0.5, occasional or rare necrotic cells in glands or crypts; Grade 1, multiple foci of necrotic cells in glands or crypts; Grade 2, necrosis involving several crypts or glands with focal abscess formation in crypts; Grade 3, widespread crypt abscesses with focal glandular destruction; Grade 4, loss of mucosa with granulation tissue response; Skin-ear: Grade 0.5, occasional or rare single basal vacuolar necrosis; Grade 1, several foci of single basal vacuolar necrosis; Grade 2, contiguous single cell necrosis or multiple necrotic cells in proximity of lymphoid infiltrates; Grade 3, confluent loss of cells with cleft formation or loss of skin appendages with extensive lymphoid infiltrates; Grade 4, loss of epidermis or epithelium with or without granulation tissue response.

Targeting JAK3 with a JAK3 Inhibitor such as WHI-P131 Inhibits Mitogenic and MLR Responses of JAK3^{+/+} Splenocytes from Wildtype C57BL/6 Mice

We hypothesized that targeting JAK3 in donor lymphocytes with a chemical inhibitor may provide an effective means to prevent severe GVHD after BMT across the MHC barrier. We first used a one-way MLR assay to measure the in vitro alloresponses of Jak3^{+/+} splenocytes from wildtype C57BL/6 mice (H-2^{b}) to mitomycin-treated splenocytes from BALB/c mice (H-2^{d}) that were used as stimulators in the presence and absence of the rationally designed JAK3 inhibitor WHI-P131. (Sudbeck E A, Liu X-P, Narla R K, et al. Structure-based design of specific inhibitors of Janus kinase 3 as apoptosis-inducing anti-leukemic agents. Clin. Cancer Res. 1999; 5:1569-1582.) As shown in FIG. 43A, WHI-P131 inhibited the MLR responses of C57BL/6 splenocytes in a concentration-dependent fashion with a near-complete inhibition at ≧1 μg/mL. WHI-P131 also inhibited the mitogenic responses of C57BL/6 splenocytes to PHA (FIG. 43B) and Con A (FIG. 1C). Notably, our previous studies have demonstrated that 10-100 μg/mL concentrations of WHI-P131 can be achieved in mice and monkeys without significant side effects. (Uckun F M, Ek O, Liu X-P, Chen C-L. In vivo toxicity and pharmacokinetic features of the Janus kinase 3 inhibitor WHI-P131 [4-(4′-hydroxyphenyl)-amino-6,7-dimethoxyquinazoline]. Clin. Cancer. Res. 1999; 5:2954-2962.) Thus, these in vitro findings show that targeting JAK3 with therapeutically achievable concentrations of WHI-P131 is an effective means to inhibit the antigen as well as mitogen responses of T-cells.

Targeting Janus Kinase 3 with a JAK3 Inhibitor such as WHI-P131 Prevents Fatal Acute Graft-Versus-Host Disease Across the Major Histocompatibility Barrier in Mice

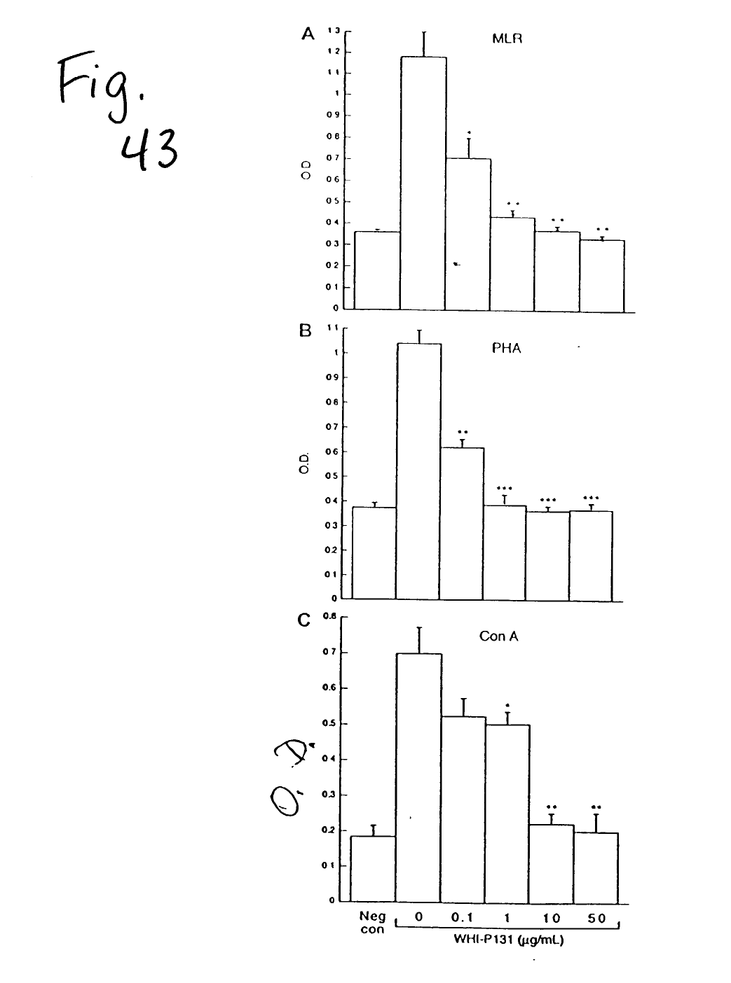

Severe GVHD was induced in lethally irradiated (7.5 Gy TBI) C57BL/6 mice (H-2^{b}) across the MHC barrier by injection of BM/S grafts from BALB/c mice (H-2^{d}). In the pilot experiment, 8 of 8 control mice developed within 3 weeks severe multiorgan GVHD, associated with overt diarrhea, hunching, weight loss, ruffled fur. Histopathologic examination of multiple organs from 5 control mice that either died or were terminated in moribund condition on or before day 30 confirmed the diagnosis of GVHD with significant liver and skin involvement (FIG. 2A.1, A.2 & A.3). The average GVHD scores in these mice were 3.1±0.2 for the liver, 2.0±0.2 for the skin, 0.9±0.2 for the small intestine, and 1.1±0.2 for the large intestine (Table 1). By comparison, none of the 8 mice treated with the JAK3 inhibitor WHI-P131 (20 mg/kg/dose, 3×/day) showed signs of severe GVHD. These mice were electively sacrificed on day 30 and the histopathologic examination of their organs showed evidence of mild-moderate GVHD (FIG. 44B.1, B.2 & B.3). The average liver and skin GVHD scores in these mice were significantly lower than those of control mice (Table 6).

TABLE 6
Attenuation of Liver and Skin GVHD by WHI-P131
and WHI-P131 + MTX

| | Time of analysis | | Organ scoring (mean ± SEM) | | | |
|---|---|---|---|---|---|---|
| Group | (weeks) | N | Liver | Skin | Small Int. | Large Int. |
| A. No Treatment | 4-6 | 5 | 3.1 ± 0.2 | 2.0 ± 0.2 | 0.9 ± 0.2 | 1.1 ± 0.2 |
| B. Vehicle | 4-6 | 10 | 2.7 ± 0.2 | 1.7 ± 0.1 | 1.1 ± 0.1 | 1.2 ± 0.2 |
| C. WHI-P131 | 4-6 | 8 | 1.6 ± 0.1*(*) | 0.9 ± 0.3*(*) | 0.8 ± 0.1 | 0.7 ± 0.1 |
| D. WHI-P131 | 12 | 3 | 0.7 ± 0.2*(*) | 0.5 ± 0.3*(*) | 0.5 ± 0.0 | 0.6 ± 0.0 |
| E. WHI-P131 + MTX | 12 | 7 | 1.5 ± 0.0*(*) | 0.2 ± 0.1*(*) | 1.1 ± 0.1 | 0.8 ± 0.1 |

Scoring was done as described in Materials and Methods. Statistical analysis of the differences between the WHI-P131- and untreated group (vehicle-treated group) was done by Student's t-test;
*(*)P<0.01.

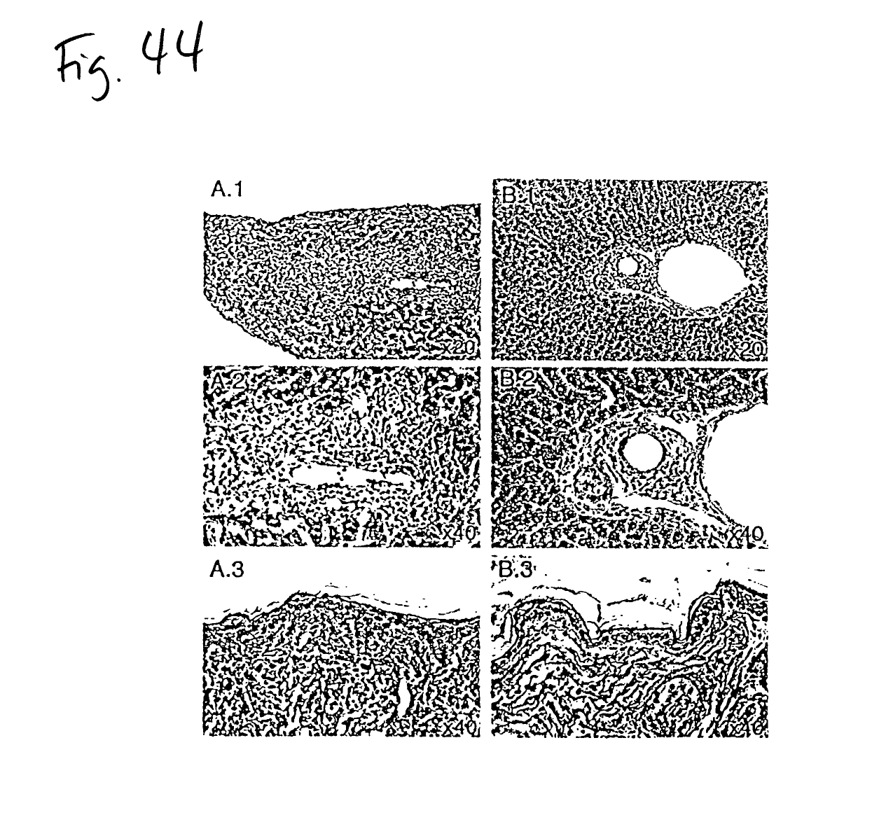

We next set out to determine the effect of WHI-P131 on post-BMT survival outcome of mice in this murine model of GVHD. In an attempt aimed at preventing the development of fatal GVHD, recipient mice were treated with WHI-P131 (20 mg/kg/dose, 3×/day) every day from the day of BMT until the end of the 85 day observation period. Control mice were treated with vehicle alone. As shown in FIG. 45 and Table 7, all of the 19 lethally irradiated C57BL/6 mice injected with syngeneic BM/S grafts remained alive throughout the 85-day observation period. By comparison, the TBI-conditioned, vehicle-treated control C57BL/6 mice (N=38) receiving BM/S grafts from BALB/c mice survived the acute TBI toxicity, but they all developed severe multiorgan GVHD, as clinically signaled by development of overt diarrhea, hunching, >20% weight loss, ruffled fur within 2-3 weeks and died with a median survival of 37 days (FIG. 45, Table 7).

TABLE 7
Attenuation of lethal GVHD in murine allogeneic BMT
recipients by targeting JAK3 with WHI-P131

| Treatment | | | umulative roportion Surviving | | | P-value | (Log Rank) |
|---|---|---|---|---|---|---|---|
| Protocol | N | MST d | 30 days | 60 days | 85 days | vs. B | vs. C |
| A | 19 | >85 | 100 ± 0 | 100 ± 0 | 100 ± 0 | — | — |
| B | 38 | 37 | 68 ± 8 | 11 ± 5 | 0 ± 0 | — | <0.0001 |
| C | 32 | 56 | 94 ± 4 | 41 ± 9 | 19 ± 7 | <0.0001 | — |
| D | 14 | 54 | 93 ± 7 | 43 ± 13 | 14 ± 9 | 0.007 | NS |
| E | 9 | 44 | 89 ± 11 | 11 ± 11 | 0 ± 0 | NS | 0.02 |
| F | 9 | 35 | 89 ± 11 | 0 ± 0 | 0 ± 0 | NS | 0.02 |
| G | 16 | 63 | 94 ± 6 | 56 ± 12 | 25 ± 11 | <0.0001 | NS |
| H | 23 | >85 | 100 ± 0 | 78 ± 9 | 70 ± 10 | <0.0001 | 0.0001 |

Treatment Protocols
A TBI + Syngeneic BMT
B TBI + Allo-BMT (H-2^{d} to H-2^{b}) + Vehicle*
C TBI + Allo-BMT (H-2^{d} to H-2^{b}) + WHI-P131
D TBI + Allo-BMT (H-2^{d} to H-2^{b}) + WHI-P131 Delayed Treatment
E TBI + Allo-BMT (H-2^{d} to H-2^{b}) + WHI-P132
F TBI + Allo-BMT (H-2^{d} to H-2^{b}) + WHI-P132 Delayed Treatment
G TBI + Allo-BMT (H-2^{d} to H-2^{b}) + MTX
H TBI + Allo-BMT (H-2^{d} to H-2^{b}) + WHI-P131 + MTX**
In two independent experiments, summarized on this table, C57BL/6 (H-2^{b}) recipients were lethally irradiated (TBI = 7.5 Gy) and transplanted with BM/S grafts from MHC-disparate BALB/c (H-2^{d}) mice and subjected to the treatment regimens presented above (the details of these treatment regimens are given above);
**P = 0.009 compared to group G; statistically signifcant difference obtained by life table analysis (log-rank test).
*The MST of the control group B was 40 days in thefirst exper first experiment involving 15 control mice and 33 days in the second experiment involving 23 control mice. The cumulative proportion of control mice surviving at 60 days was 13 ± 9% in the first experiment and 9 ± 6% in the second experiment. No control mouse was alive at 85 days in either of these two experiments.

Histopathologic examination of multiple organs from moribund mice that were electively sacrificed between 4 and 6 weeks post-BMT (N=10) confirmed the diagnosis of GVHD. The average GVHD scores were 2.7±0.2 for the liver, 1.7±0.1 for the skin, 1.1±0.1 for the small intestine, and 1.2±0.2 for the large intestine (Table 6).

Notably, WHI-P131 treatment significantly improved the survival of BMT recipients (P<0.0001, log-rank test) and prolonged the median survival time (MST) to 56 days (Table 7). The probability of survival at 2 months post-BMT was 11±5% for vehicle-treated control mice and 41±9% for mice treated with WHI-P131 (FIG. 45, Table 7). The standard anti-GVHD drug MTX (10 mg/m²/day once daily) was used for comparison. WHI-P131 was comparable to MTX in its efficacy to improve the survival outcome post-BMT (Table 7). The histopathologic examination of the organs from the WHI-P131-treated long-term survivors (N=3) that were electively sacrificed on day 85 post-BMT revealed evidence for subclinical mild GVHD involving the liver, small/large intestine, and skin. According to the scoring system, the GVHD grades were 0.7±0.2 for the liver, 0.5±0.0 for the small intestine, 0.6±0.0 for the large intestine, and 0.5±0.3 for the skin (Table 6).

We next asked if WHI-P131 could still improve the survival outcome if the therapy is delayed until the 3rd week (day 18) after BMT. As shown in Table 7, this treatment also resulted in significant improvement of survival with a 43±13% probability of survival at 2 months and a median survival of 54 days. In contrast to WHI-P131, the structurally related control dimethoxyquinazoline compound WHI-P132, which does not inhibit JAK3, did not improve the survival after BMT even when its administration commenced on the day of BMT (Table 7). Taken together, these results indicate that targeting JAK3 in alloreactive donor lymphocytes with a chemical inhibitor such as WHI-P131 may improve survival after BMT across the MHC barriers by decreasing the probability of fatal GVHD.

Efficacy of WHI-P131 Plus Methotrexate Combination in Prevention of Fatal Acute Graft-Versus-Host Disease Across the Major Histocompatibility Barrier in Mice.

We next sought to identify an effective GVHD prevention regimen which employs the JAK3 inhibitor WHI-P131 in combination with an immunosuppressive agent. As shown in Table 7 and FIG. 45, the combination regimen WHI-P131 plus MTX was more effective than WHI-P131 alone or MTX alone. More than half of the C57BL/6 recipients receiving this most effective GVHD prophylaxis remained alive and healthy throughout the 85-day observation period with a cumulative survival probability of 70±10% (Table 7, FIG. 45).

The longterm survival of WHI-P131-, MTX-, or WHI-P131+MTX-treated mice was not due to poor engraftment of donor cells (Table 8).

TABLE 8
Donor Cell Engraftment

| Group | N | H-2D^{d} (%) |
|---|---|---|
| Vehicle | 5 | 94.7 ± 1.4 |
| WHI-P131 | 3 | 94.5 ± 2.3 |
| MTX | 3 | 97.6 ± 1.0 |
| WHI-P131 + MTX | 7 | 97.0 ± 2.0 |
| Syngeneic Controls | 3 | 0.0 ± 0.0 |

Vehicle treated mice were analyzed at the time of death between 50 and 77 days. All other mice were electively sacrificed and analyzed at the end of the 85 day observation period. Data are presented as mean ± SEM values for the percentage of H-2D^{d} positive donor cells in the nucleated peripheral blood cell population.

Notably, 94.5±2.3% H-2D^{d}-positive donor cell engraftment was observed in WHI-P131-treated mice (Table 8), indicating that WHI-P131 does not impair the ability of donor T-cell to prevent graft rejection. At the end of the observation period, flow cytometric H-2D^{d}-typing of nucleated peripheral blood cells obtained from 7 representative mice in the WHI-P131+MTX treatment group showed 97±2% H-2D^{d}-positive donor cell engraftment (Table 8). Although these long-term surviving allograft recipient C57BL/6 mice showed no clinical evidence of GVHD, the histopathologic examination of their organs revealed evidence for subclinical mild GVHD involving the liver, small/large intestine, and skin. According to the scoring system, the histologic GVHD grades were 1.5±0.0 for the liver, 1.1±0.1 for the small intestine, 0.8±0.1 for the large intestine, and 0.2±0.1 for the skin (Table 6).

Graft-versus-host disease (GVHD) and its complications significantly limit the success of allogeneic and unrelated BMT for patients with acute lymphoblastic leukemia (ALL), acute myeloblastic leukemia (AML), and chronic myelocytic leukemia (CML). (Apperly JF, Jones L, Hale G, et al. Bone marrow transplantation for patients with chronic myeloid leukemia: T-cell depletion with Campath-1 reduces the incidence of graft-versus-host disease but may increase the risk of leukemic relapse. Bone Marrow Transplant. 1986; 1:53-60; Wingard J R, Piantadosi S, Santos G W, et al. Allogeneic bone marrow transplantation for patients with high-risk acute lymphoblastic leukemia. J Clin Oncol. 1990; 8:820-830; Thomas E D, Clift R A. Indications for marrow transplantation in chronic myelogenous leukemia. Blood. 1989; 73:861-864; Barrett A J, Horowitz M M, Gape R P, et al. Marrow transplantation for acute lymphoblastic leukemia: factors affecting relapse and survival. Blood. 1989; 74:862-871; and Clift R A, Buckner C D, Appelbaum F R, et al. Allogeneic marrow transplantation in patients with acute myeloid leukemia in first remission: A randomized trial of two irradiation regimens. Blood. 1990; 76:1867-1871). While the prevention and treatment of GVHD are essential for a successful outcome of BMT, absence of GVHD has been associated with an increased risk of relapse in leukemia patients (especially CML patients) undergoing BMT due to the lack of graft-versus-leukemia (GVL) effects against residual host leukemia cells. (Apperly J F, Jones L, Hale G, et al. Bone marrow transplantation for patients with chronic myeloid leukemia: T-cell depletion with Campath-1 reduces the incidence of graft-versus-host disease but may increase the risk of leukemic relapse. Bone Marrow Transplant. 1986; 1:53-60). Furthermore, for certain leukemia patients undergoing BMT, especially those with high risk ALL, recurrence of leukemia still remains the major cause of failure. (Wingard J R, Piantadosi S, Santos G W, et al. Allogeneic bone marrow transplantation for patients with high-risk acute lymphoblastic leukemia. J Clin Oncol. 1990; 8:820-830; and Dinsmore R, Kirkpatrick D, Flomenberg N, et al. Allogeneic bone marrow transplantation for patients with acute nonlymphocytic leukemia. Blood. 1983; 62:381-388). Drugs used for GVHD prevention are CSA and MTX. (Leelasiri A, Greer J P, Stein RS, et al. Graft-versus-host disease prophylaxis for matched unrelated donor bone marrow transplantation: comparison between cyclosporine-methotrexate and cyclosporine-methotrexate-methylprednisolone. Bone Marrow Transplant. 1995; 15:401-405; Ruutu T, Volin L, Parkkali T, Juvonen E, Elonen E. Cyclosporine, methotrexate, and methylprednisolone compared with cyclosporine and methotrexate for the prevention of graft-versus-host disease in bone marrow transplantation from HLA-identical sibling donor: a prospective randomized study. Blood. 2000; 96:2391-2398; Storb R, Pepe M, Anasetti C, et al. What role for prednisone in prevention of graft-versus-host disease in patients undergoing marrow transplants. Blood. 1990; 76:1037-1045; and Atkinson K, Biggs J, Concannon A, et al. A prospective randomized trial of cyclosporine and methotrexate versus cyclosporine, methotrexate and prednisolone for prevention of graft-versus-host disease after HLA-identical sibling marrow transplantation for hematological malignancy. Aust N A J. Med. 1991; 21:850-856). PDN has been widely used for treatment of GVHD and its use for prevention of GVHD is still under investigation. (Leelasiri A, Greer J P, Stein R S, et al. Graft-versus-host disease prophylaxis for matched unrelated donor bone marrow transplantation: comparison between cyclosporine-methotrexate and cyclosporine-methotrexate-methylprednisolone. Bone Marrow Transplant. 1995; 15:401-405; Ruutu T, Volin L, Parkkali T, Juvonen E, Elonen E. Cyclosporine, methotrexate, and methylprednisolone compared with cyclosporine and methotrexate for the prevention of graft-versus-host disease in bone marrow transplantation from HLA-identical sibling donor: a prospective randomized study. Blood. 2000; 96:2391-2398; Storb R, Pepe M, Anasetti C, et al. What role for prednisone in prevention of graft-versus-host disease in patients undergoing marrow transplants. Blood. 1990; 76:1037-1045; Atkinson K, Biggs J, Concannon A, et al. A prospective randomized trial of cyclosporine and methotrexate versus cyclosporine, methotrexate and prednisolone for prevention of graft-versus-host disease after HLA-identical sibling marrow transplantation for hematological malignancy. Aust N A J. Med. 1991; 21:850-856; and Deeg H J, Lin D, Leisenring W, et al. Cyclosporine and cyclosporine plus methylprednisolone for prophylaxis of graft-versus-host disease: a prospective randomized trials. Blood 1997; 89:3880-3887.) Of these 3 drugs, only PDN and MTX have antileukemic activity against ALL cells and none exhibits activity against AML or CML cells. Furthermore, front-line use of steroids and MTX in ALL patients is associated with steroid resistance as well as MTX resistance at relapse. (Matherly L H, Taub J W. Methotrexate pharmacology and resistance in childhood acute lymphoblastic leukemia. Leuk Lymphoma 1996; 21:359-366). Therefore, the currently available drugs for GVHD prevention or treatment are not expected to have a substantial positive impact on leukemic relapse rates after BMT. Therefore, dual-function immunosuppressive agents with potent antileukemic activity are urgently needed. Such agents could reduce the risk of both severe GVHD and leukemic relapse after BMT.

Our earlier studies have shown that JAK3 is a vital target in human leukemia cells and demonstrated that the rationally designed JAK3 inhibitor WHI-P131 triggers apoptosis in lymphoblastic as well as myeloblastic leukemia cells. (Sudbeck E A, Liu X-P, Narla R K, et al. Structure-based design of specific inhibitors of Janus kinase 3 as apoptosis-inducing anti-leukemic agents. Clin. Cancer Res. 1999; 5:1569-1582). WHI-P131 was very well tolerated by cynomolgus monkeys and plasma concentrations of WHI-P131 that are cytotoxic to human leukemia cells in vitro could be achieved in monkeys at non-toxic dose levels. (Uckun FM, Ek O, Liu X-P, Chen C-L. In vivo toxicity and pharmacokinetic features of the Janus kinase 3 inhibitor WHI-P131 [4-(4′-hydroxyphenyl)-amino-6,7-dimethoxyquinazoline]. Clin. Cancer. Res. 1999; 5:2954-2962). Intriguingly, JAK3 is not required for production of myeloid precursors in bone marrow (Grossman W J, Verbsky J W, Yang L, et al. Dysregulated myelopoiesis in mice lacking Jak3. Blood. 1999; 94:932-939) and WHI-P131 did not cause any myelosuppression in mice or monkeys. (Uckun F M, Ek O. Liu X-P, Chen C-L. In vivo toxicity and pharmacokinetic features of the Janus kinase 3 inhibitor WHI-P131 [4-(4′-hydroxyphenyl)-amino-6,7-dimethoxyquinazoline]. Clin. Cancer. Res. 1999; 5:2954-2962). The antileukemic activity and lack of significant systemic toxicity of WHI-P131 suggested that this JAK3 inhibitor may be useful in the treatment of relapsed or therapy-refractory leukemia patients. One of the purposes of the present study was to examine the effectiveness of targeting JAK3 with WHI-P131 for prevention and treatment of lethal GVHD across major histocompatibility barrier in mice. Here, we show that WHI-P131 exhibits potent in vivo biologic activity in an aggressive acute GVHD model using BALB/c (H-2^{d}) donor bone marrow/spleen cells and H-2 disparate C57BL/6 (H-2^{b}) recipient mice. TBI-conditioned, vehicle-treated control C57BL/6 mice receiving bone marrow/splenocyte grafts from BALB/c mice survived the acute TBI toxicity, but they all developed histologically confirmed severe multiorgan GVHD and died with a median survival of 37 days. WHI-P131 treatment significantly improved the survival outcome of the BMT recipients even when the therapy was delayed until the 3rd week after BMT. Our present study indicates that this JAK3 inhibitor could be useful as a dual-function anti-GVHD agent with potent anti-leukemic activity. Notably, the combination regimen WHI-P131 plus MTX (10 mg/m²/day) was more effective than WHI-P131 alone or MTX alone. More than half of the C57BL/6 recipients receiving this most effective GVHD prophylaxis remained alive throughout the 85-day observation period.

Taken together, these results indicate that targeting JAK3 in alloreactive donor lymphocytes with a chemical inhibitor such as WHI-P131 may be useful in prevention of severe GVHD after BMT. The H-2D typing of nucleated peripheral blood cells from WHI-P131-treated mice confirmed that >90% of the circulating cells are of donor origin. These results indicate that WHI-P131 does not impair the ability of donor T-cells in the graft to prevent rejection. Therefore, no additional immunosuppression would seem necessary to ensure longterm engraftment in the context of WHI-P131 therapy for GVHD prophylaxis.

### Example 10

### Mice.

Female BALB/cJ (H-2^{d}), C57BL/6J (B6) (H-2^{b}) and CB6F1/J (BALB/cJxC57BL/6J) (F1) (H-2^{d/b}) mice were purchased from the Jackson Laboratory (Bar Harbor, Me.) at 6-8 weeks of age. Mice were housed in a controlled environment (12-h light/12-h dark photoperiod, 22±1° C., 60±10% relative humidity), which is fully accredited by the USDA (United States Department of Agriculture). All husbandry and experimental procedures were performed under specific pathogen-free (SPF) conditions. All mice were kept in Micro-Isolator cages (Lab Products, Inc., Maywood, N.Y.) containing autoclaved food (LM-485, Harlan Teklad, Madison, Wis.), water and bedding. Animal studies were approved by the Parker Hughes Institute Animal Care and Use Committee, and all animal care procedures conformed to the Principles of Laboratory Animal Care (NIH publication #85-23, revised 1985).

Total Body Irradiation (TBI) and Allogeneic Bone Marrow Transplantation (BMT).

For pre-BMT conditioning, recipient F1 mice, positioned in a pie-shaped Lucite holder (Braintree Scientific Inc., Boston, Mass.) underwent TBI (9.5 Gy) one day prior to BMT, which was delivered by a Cesium Instrument (J L Sheppard Labs, 64.38 rad/min). Recipients were given antibiotic-supplemented water (sulfamethoxazole/trimethoprim, Hi-Tech Pharmacal, Amityville, N.Y.) starting the day before BMT. Donor (B6) bone marrow (BM) was collected into RPMI 1640 medium supplemented with L-glutamine (Cellgro) (Mediatech, Hendon, Va.) by flushing the shafts of femurs and tibias, as described previously 20. In brief, BM cells were suspended by agitation with a pasteur pipette and separated from debris by passing through a fine pore nylon cell strainer. Red blood cells were eliminated by lysis buffer and clumps of debris were allowed to settle out. In parallel, single cell suspensions of B6 splenocytes (S) were prepared, (Cetkovic-Cvrlje M, Roers B A, Waurzyniak B, Liu X P, Uckun F M. Targeting Janus Kinase 3 to attenuate the severity of acute graft-versus-host disease across the major histocompatibility barrier in mice. Blood) as a source of GVHD-causing T-cells. The standard BM/S inoculum consisted of 25×10⁶ BM cells and 25×10⁶ splenocytes in 0.5 ml of RPMI 1640 and was injected intravenously (i.v.) into the caudal vein.

WHI-P131 Treatment.

WHI-P131 [4-(4′-hydroxyphenyl)-amino-6,7-dimethoxyquinazoline] was synthesized and characterized as previously described in detail¹⁸. WHI-P131 was administered in 200 μL PBS supplemented with 10% DMSO as the vehicle, as previously reported. (Cetkovic-Cvrlje M, Roers B A, Waurzyniak B, Liu X P, Uckun F M. Targeting Janus Kinase 3 to attenuate the severity of acute graft-versus-host disease across the major histocompatibility barrier in mice. Blood). Control mice were treated daily by vehicle (200 μL PBS supplemented with 10% DMSO). WHI-P131 (50 mg/kg/day in 2 divided doses) was administered daily via intraperitoneal (i.p) injections to BMT recipient mice for prevention of fatal GVHD starting on the day of BMT/BCL-1 injection (day 0) until day 60 or an earlier elective sacrifice date due to the moribund clinical condition of the mouse. BALB/c mice injected with BCL-1 cells were treated daily by 75 mg/kg/day (in 3 divided doses) and 150 mg/kg/day (in 3 divided doses) WHI-P1131 starting on the day of the BCL-1 inoculation until the death or elective sacrifice of the mouse.

Graft-Versus-Host Disease (GVHD) Monitoring.

BMT recipients were monitored daily for any clinical evidence of GVHD (weight loss, manifestations of skin erythema, alopecia, hunching, diarrhea) and survival during the 60-day observation period. Survival times were measured from the day of BMT (day 0).

BCL-1 Leukemia Model and Syngeneic BMT

BCL-1 cells were isolated from the spleen of a female BALB/c (H-2^{d}) mouse with a spontaneously arising B-cell leukemia, which was first described by Slavin and Strober. (Slavin S, Strober S. Spontaneous murine B-cell leukemia. Nature. 1978; 272:624-626). The parent BCL-1 cell line for the present study was generously provided by Drs. Jonathan Uhr and Ellen Vitteta from the University of Texas Health Sciences Center (Dallas, Tex.). (Krolick K A, Isakson P C, Uhr J W, Vitetta E S. Murine B cell leukemia (BCL-1): organ distribution and kinetics of growth as determined by fluorescence analysis with an anti-idiotypic antibody. J. Immunol. 1979; 123:1928-1935). In experiments presented herein, BCL-1 cells were harvested and processed as described previously. (Uckun F M, Chelstrom L M, Irvin J D, et al. In vivo efficacy of B43 (anti-CD 19)-pokeweed antiviral protein immunotoxin against BCL-1 murine B-cell leukemia. Blood. 1992; 15:2649-2661; and Waddick K G, Finnegan D M, Chelstrom L M, Uckun F M. In vivo radiosensitizing effects of recombinant interleukin 6 on radiation resistant BCL-1 B-lineage leukemia cells in a murine syngeneic bone marrow transplantation model system. Leuk Lymphoma. 1995; 19:121-128. Recipient F1 mice received TBI of 9.5 Gy on day −1 (as described above) before inoculation of leukemic cells and syngeneic BMT. Syngeneic donor (F1) BM (25×10⁶) and spleen cells (25×10⁶) were prepared as described above and injected i.v. to F1 recipients in parallel with a BCL-1 cell inoculum (5×10⁶ cells) on day 0. Survival of mice was monitored by daily observation, and the day of death was recorded as the day the mouse spontaneously died or was sacrificed in moribund condition.

GVL Model.

BCL-1 leukemia cells (5×10⁶) were injected intravenously into TBI-conditioned (day −1) F1 recipients in parallel with an allogeneic marrow graft (B6 BM/S cells, 25×10⁶ each) on day 0 of BMT. Survival was monitored daily, and the cause of death for each mouse was determined by postmortem gross and histopathological examination. Leukemic deaths were characterized by the presence of massive splenomegaly and absence of GVHD-associated histopathologic changes in skin, liver, and intestine. GVHD deaths were discernible from leukemic deaths by the absence of the clinical signs of end-stage leukemia (i.e., visible/palpable splenomegaly) combined with the presence of GVHD-asociated clinical signs (i.e., weight loss, manifestations of skin erythema, allopecia, hunching, diarrhea) at the time of death along with a small spleen size (<0.2 g) as well as detection of GVHD-associated histopathologic changes in skin, liver, and intestine during the postmortem examination.

H-2D Typing by Multiparameter Flow Cytometry.

The engraftment status of WHI-P131-treated BMT recipients was studied by two-color flow cytometric H-2D^{d} typing of splenocytes using PE-labeled anti-H-2D^{b} antibody (clone KH95, Pharmingen, San Diego, Calif.) which marks C57BL/6-type cells and FITC-labeled anti-H-2D^{d} antibody (clone 34-2-12, Pharmingen) which marks BALB/c-type cells. The leukemic burden was studied by two-color flow cytometry of splenic lymphocyte subpopulations using the PE-labeled anti-H-2D^{b} antibody in combination with FITC-labeled B-cell reactive anti-B220 (clone RA3-6B2, Pharmingen) antibody. Immunofluorescent staining of cells and multiparameter flow cytometry were performed using standard procedures and a FACSCaliber instrument (Becton Dickinson, Mountain View, Calif.).

Histopathology.

Spleen, liver, skin and gut were harvested from mice at the time of death. Tissues were fixed in 10% buffered formalin, paraffin embedded, cut into 4 μm sections and stained with hematoxylin and eosin. All histopathology slides were blindly examined by a certified veterinary pathologist (B.W.). The presence of leukemic BCL-1 infiltrates in the liver and spleens of mice challenged with a BCL-1 inoculum was histopathologically confirmed, as described previously. (Uckun F M, Chelstrom L M, Irvin J D, et al. In vivo efficacy of B43 (anti-CD19)-pokeweed antiviral protein immunotoxin against BCL-1 murine B-cell leukemia. Blood. 1992; 15:2649-2661). Livers were scored positive for GVHD when there was a periportal infiltrate with acute necrosis, skin was scored positive when there was single cell necrosis, and colon was scored positive when there was single cell necrosis or crypt dropout. After initial scoring, all slides were reviewed for GVHD grading, as described. Our GVHD grading system was as follows: Liver: Grade 0.5, focal portal lymphoid infiltrate; Grade 1, widespread portal lymphoid infiltrate 1; Grade 2, focal bile duct invasion or cellular injury; Grade 3, multiple foci of bile duct injury and regeneration; Grade 4, widespread injury and destruction of bile ducts; Small and large intestine: Grade 0.5, occasional or rare necrotic cells in glands or crypts; Grade 1, multiple foci of necrotic cells in glands or crypts; Grade 2, necrosis involving several crypts or glands with focal abscess formation in crypts; Grade 3, widespread crypt abscesses with focal glandular destruction; Grade 4, loss of mucosa with granulation tissue response; Skin-ear: Grade 0.5, occasional or rare single basal vacuolar necrosis; Grade 1, several foci of single basal vacuolar necrosis; Grade 2, contiguous single cell necrosis or multiple necrotic cells in proximity with lymphoid infiltrate; Grade 3, confluent loss of cells with cleft formation or loss of skin appendages with heavy lymphoid infiltrate; Grade 4, loss of epidermis or epithelium with or without granulation tissue response.

Detection of Residual BCL-1 Cells by Adoptive Transfer Experiments.

In order to determine whether or not residual BCL-1 cells were present in BMT recipients, 5×10⁵ or 1×10⁶ spleen cells obtained from F1 recipients 11 days, 23 days or >60 days post BMT/BCL-1 inoculation were adoptively transferred to untreated secondary syngeneic (BALB/c) recipients via i.v. injection into the caudal vein. Leukemia-free survival for at least 100 days in secondary syngeneic recipients was indicative of absence of residual leukemia in BMT recipients since as few as 10 BCL-1 cells cause leukemic death within 12 weeks and 100 BCL-1 cells cause leukemic death within 6 weeks (Krolick K A, Isakson P C, Uhr J W, Vitetta E S. Murine B cell leukemia (BCL-1): organ distribution and kinetics of growth as determined by fluorescence analysis with an anti-idiotypic antibody. J. Immunol. 1979; 123:1928-1935; Uckun F M, Chelstrom L M, Irvin J D, et al. In vivo efficacy of B43 (anti-CD19)-pokeweed antiviral protein immunotoxin against BCL-1 murine B-cell leukemia. Blood. 1992; 15:2649-2661; and Slavin S, Weiss L, Morecki S, Ben Bassat H, Leizerowitz R. Ultrastructural, cell membrane and cytogenetic characteristics of B-cell leukemia (BCL-1), a murine model of chronic lymphocytic leukemia. Cancer Res. 1981; 41:4162-4166).

Statistical Analysis.

Group comparisons of continuous variables were done using Student's t-tests. The survival data were analyzed by life-table methods. P-values of less than 0.05 (log-rank test) were considered significant.

BCL-1 Cells Cause Fatal Leukemia in BALB/c Mice and Irradiated (BALB/cJxC57BL/6J)F1 Mice Undergoing Syngeneic BMT.

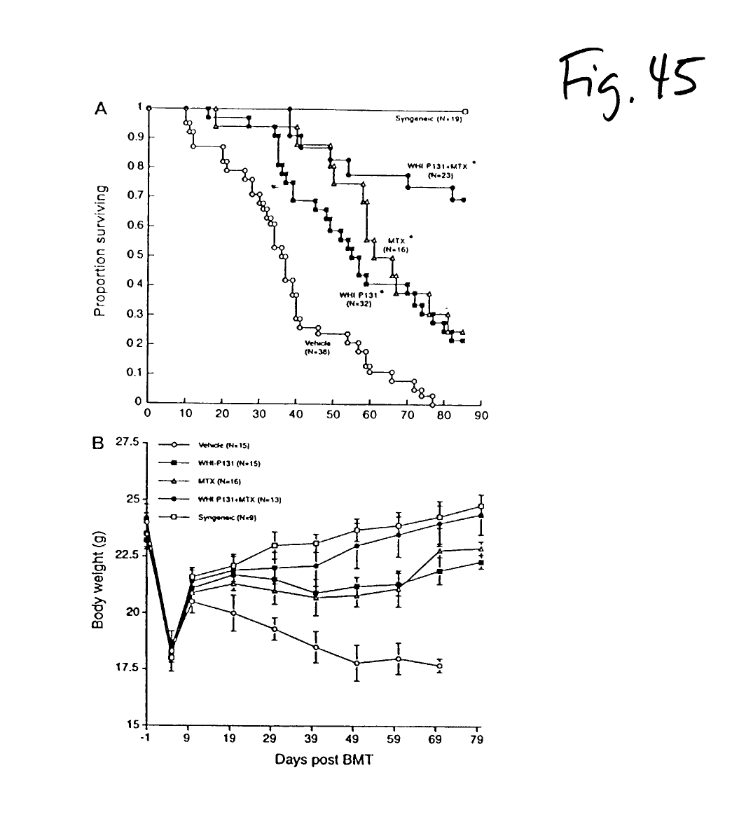

An intravenous inoculum containing 1×10⁶ BCL-1 cells caused fatal leukemia in 100% of control BALB/c mice (N=20) with a median survival time of only 12 days (FIG. 46A). At dose levels of 75 mg/kg/day (divided in three doses) (N=20) or 150 mg/kg/day (divided in three doses) (N=20) which are 1.5- to 3-times higher than the effective 50 mg/kg/day anti-GVHD dose. WHI-PI31 exhibited no detectable antileukemic activity against BCL-1 leukemia cells (FIG. 46A). The median survival times were 11 days for the 75 mg/kg dose level and 12 days for the 150 mg/kg dose level. All 20 control and 40 WHI-P131-treated BALB/c mice had massive splenomegaly at the time of death and histopathological examination of their liver and spleen showed diffuse effacement of the normal architecture with leukemic cells.

We next examined the ability of BCL-1 cells to cause fatal leukemia in irradiated F1 recipients undergoing syngeneic BMT. An intravenous inoculum of 5×10⁶ BCL-1 cells caused fatal leukemia in 100% of control F1 recipients (N=22) with a median survival time of 14 days (FIG. 46B, Table 1). Thus, any microenvironmental changes caused by TBI and/or BMT did not prevent BCL-1 cells from engrafting and expanding in the organs of F1 mice. In accordance with the BALB/c data, WHI-P131 (50 mg/kg/day) did not exhibit any detectable antileukemic activity against BCL-1 cells in irradiated F1 recipients undergoing syngencic BMT. All 13 WHI-P131-treated F1 recipients died of leukemia with a median survival time of 12 days (FIG. 46B, Table 9). All 22 control and 13 WHI-P131-treated F1 mice had massive splenomegaly at the time of death and histopathological examination of their liver and spleen showed diffuse effacement of the normal architecture with leukemic cells.

TABLE 9
Effects of the JAK-3 Inhibitor WHI-P131 on Post-BMT
Survival Outcome in Murine GVHD and GVL Models

| | | | Cumulative | | | |
|---|---|---|---|---|---|---|
| Treatment | | MST | Proportion Surviving | | P-value | P-value |
| Protocol | N | days | 30 days | 60 days | vs. E | vs. C |
| A | 5 | 11 | 0 ± 0 | 0 ± 0 | | |
| B | 5 | 60 | 100 ± 0 | 100 ± 0 | | |
| C | 22 | 14 | 14 ± 7 | 0 ± 0 | 0.04 | — |
| D | 13 | 12 | 8 ± 7 | 0 ± 0 | 0.01 | N.S. |
| E | 23 | 24 | 9 ± 6 | 0 ± 0 | — | 0.04 |
| F | 17 | 37 | 60 ± 12 | 29 ± 11 | <0.0001 | <0.0001 |
| G | 28 | 25 | 11 ± 6 | 4 ± 4 | N.S. | 0.01 |
| H | 16 | 36 | 63 ± 12 | 44 ± 12 | <0.0001 | <0.0001 |

Treatment Protocols
A TBI
B TBI + Syngeneic BMT -> Vehicle
C TBI + Syngeneic BMT + BCL-1 -> Vehicle
D TBI + Syngeneic BMT + BCL-1 -> WHI-P131
E TBI + Allo BMT -> Vehicle
F TBI + Allo BMT -> WHI-P131
G TBI + Allo BMT + BCL-1 -> Vehicle
H TBI + Allo BMT + BCL-1 -> WHI-P131
(BALB/cJxC57BL/6J)F1 (H-2^{d/b}) recipients were lethally irradiated (9.5 Gy) and transplanted with BM/S grafts from syngeneic F1 or allogeneic C57BL/h (H-2^{b}) donors. Some of the recipients were injected with BCL-1 cells (5 × 10⁶, i.v.) to establish GVL model and/or subjected to the WHI-P1313 (50 mg/kg/day) or Vehicle treatment;
*P<0.0001 between the group G and H; Statistically significant differences obtained by life table analysis (log-rank test).

Targeting Janus Kinase 3 with the Chemical Inhibitor WHI-P131 Prevents Fatal Acute Graft-Versus-Host Disease Across the Major Histocompatibility Barrier in (BALB/cJxC57BL/6J)F1 Mice

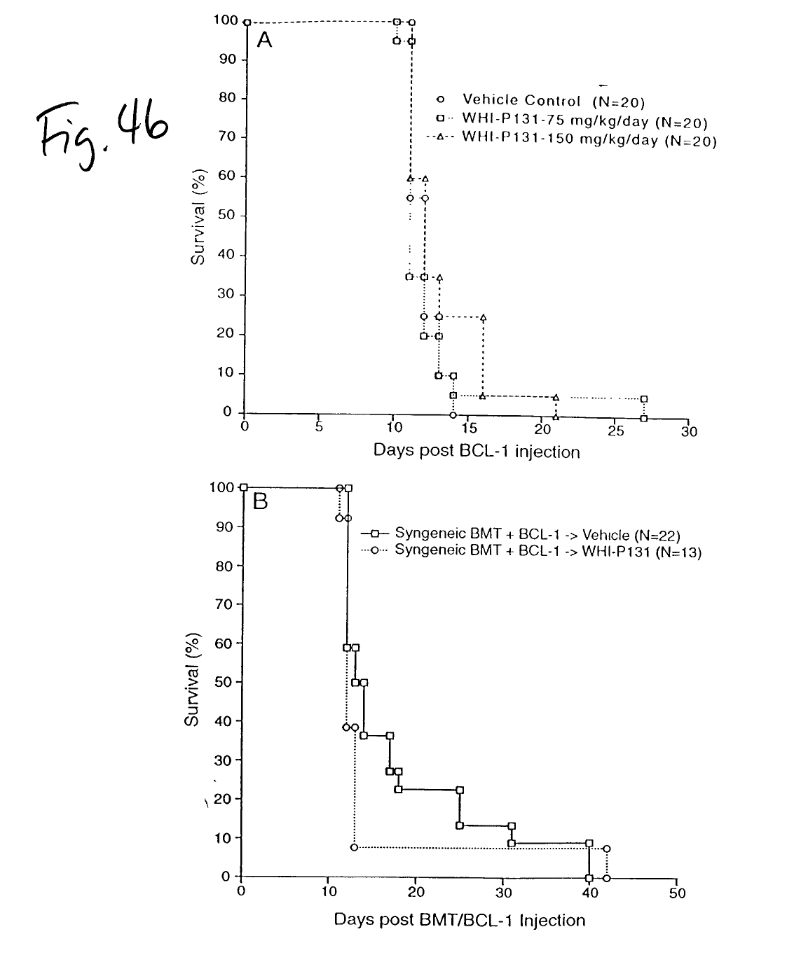

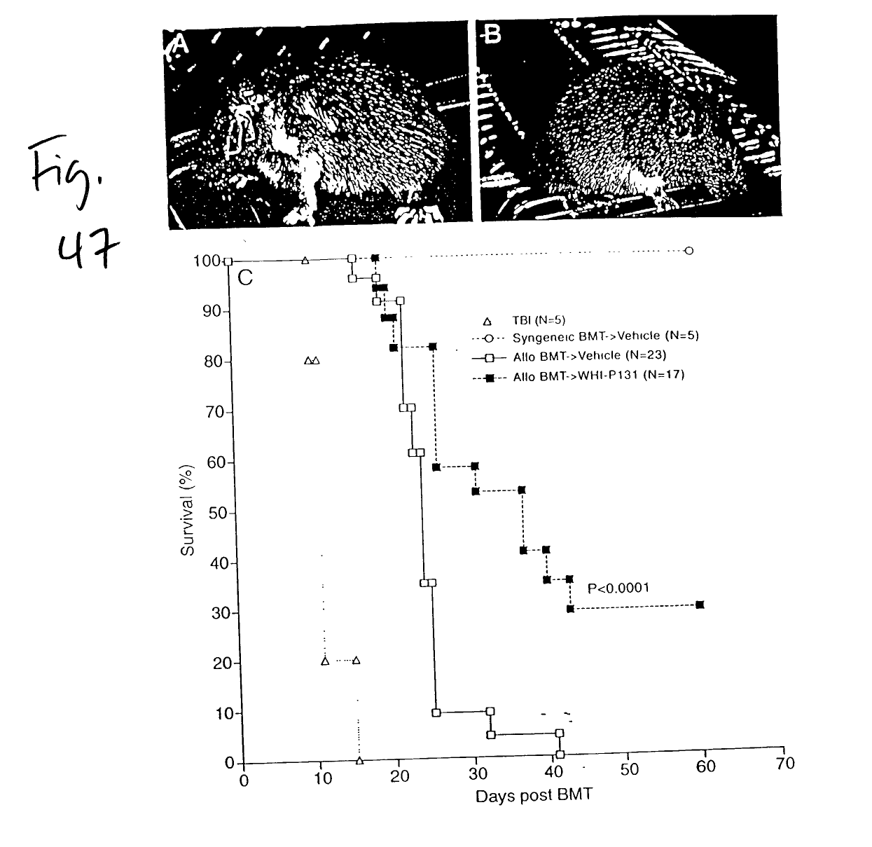

Severe GVHD was induced in lethally irradiated F1 mice (H-2^{d/b}) across the MHC barrier by injection of BM/S grafts from C57BL/6 mice (H-2^{b}). In an attempt aimed at preventing the development of fatal GVHD in this model, recipient mice were treated with WHI-P131 (25 mg/kg/dose, 2×/day) every day from the day of BMT until the end of the 60 day observation period, whereas control mice were treated with vehicle alone. As shown in Table 9 and FIG. 47C, all 5 lethally irradiated F1 mice which did not receive any BM/S grafts died of acute radiation toxicity with a median survival of 11 days, whereas all of the 5 lethally irradiated F1 mice injected with syngeneic BM/S grafts remained alive throughout the 60 day observation period. By comparison, the TBI-conditioned, vehicle-treated control F1 mice (N=23) receiving BM/S grafts from C57BL/6 mice survived the acute TBI toxicity, but they all developed severe multiorgan GVHD, as evidenced by development of overt diarrhea, hunching, >20% weight loss, alopecia, and ruffled fur. One-hundred % of these control mice died with a median survival of 24 days (Table 9, FIG. 47C). Histopathologic examination of multiple organs from randomly picked vehicle-treated mice (N=6) confirmed the diagnosis of GVHD (Table 2). The average GVHD scores were 2.8±10.1 for the liver, 1.8±0.1 for the skin, 0.8±0.1 for the small intestine, and 1.2±0.4 for the large intestine.

GVHD prophylaxis with WHI-P131 (25 mg/kg/dose, 2×/day, days 0-60) significantly improved the survival of BMT recipients and prolonged the MST to 37 days (Table 9, FIG. 47C). The probability of survival at 30 days post-BMT was 9±6% for vehicle-treated control mice (N=23) and 60±12% for mice treated with WHI-P131 (N=17) (P<0.0001, Table 9). The histopathologic examination of the organs from four representative WHI-P131-treated long-term (>8 weeks) survivors revealed evidence for subclinical mild GVHD in the liver, skin, small intestine and large intestine. According to the scoring system, the GVHD grades were 1.0±0.3 for the liver, 0.5±0.0 for the skin, 0.5±0.0 for the small intestine and 0.1±0.1 for the large intestine (Table 10). Flow cytometric H-2D^{d/b}-typing of splenocytes obtained from these four long-term survivors of WHI-P131-treated group showed 96.3±1.1% H-2D^{d-/b+} donor cell engraftment (Table 11), indicating that WHI-P131 does not prevent donor cell engraftment under these experimental conditions and the attenuation of GVHD in WHI-P131-treated recipient mice was not due to lack of donor cell engraftment with concomitant autologous recovery.

TABLE 10
Severity of GVHD in BMT Recipients

| | | | GVHD Score (mean + SEM) | | | |
|---|---|---|---|---|---|---|
| | Time of Analysis | | | | Small | Large |
| Group | (weeks) | n | Liver | Skin | Intestine | Intestine |
| A | 2-4 | 6 | 2.8 ± 0.1 | 1.8 ± 0.1 | 0.8 ± 0.1 | 1.2 ± 0.4 |
| B | >8 | 4 | 1.0 ± 0.3** | 0.5 ± 0.0*** | 0.5 ± 0.0 | 0.1 ± 0.1* |
| C | 2-4 | 9 | 2.6 ± 0.1 | 1.5 ± 0.2 | 0.8 ± 0.1 | 1.2 ± 0.3 |
| D | >8 | 4 | 0.6 ± 0.1*** | 0.0 ± 0.0** | 0.3 ± 0.1* | 0.1 ± 0.1* |
| E | 2-4 | 17 | 0.5 ± 0.0 | 0.2 ± 0.1 | 0.3 ± 0.1 | 0.2 ± 0.1 |
| F | >8 | 5 | 0.5 ± 0.0 | 0.0 ± 0.0 | 0.1 ± 0.1 | 0.0 ± 0.0 |

Group
A Allogeneic BMT -> Vehicle
B Allogeneic BMT -> WHI-P131
C Allogeneic BMT + BCL-1 -> Vehicle
D Allogeneic BMT + BCL-1 -> WHI-P131
E Syngeneic BMT (control)
F Syngeneic BMT (control)
Scoring was done as described above. Statistical analysis of the differences between the groups (B vs. A and D vs. C) was done by Student's t-test;
*P < 0.05,
**P < 0.005,
***P < 0.0001.

TABLE 11
Donor Cell Engraftment

| Group | N | H-2D^{d-/b+} (%) |
|---|---|---|
| Allogeneic BMT -> WHIP-P131 | 4 | 96.3 ± 1.1 |
| Allogeneic BMT + BCL-1-> WHI-P131 | 4 | 95.3 ± 0.8 |
| Syngeneic BMT | 5 | 0.0 ± 0.0 |

Splenocytes of long-term survivors (>60 days post BMT/BCL-1 injection), WHI-P131-treated F1 recipients of allogeneic (B6) BM/S grafts and BCL-1 injections were analyzed by flow cytometry for engraftment. Syngeneic (F1 to F1) BM/S recipients are presented as controls. Data are presented as mean ± SEM values for the percentage of H-2D^{d-/b+} donor cells.

These findings confirm and extend our previous studies which demonstrated the beneficial effects of WHI-P131 on the post-BMT survival outcome of B6 (H-2^{b}) recipients transplanted with BM/S grafts from allogeneic BALB/c (H-2^{d}) donors. Thus, targeting JAK3 in alloreactive donor lymphocytes with a chemical inhibitor such as WHI-P131 can improve survival after BMT across the MHC barriers by decreasing the probability of fatal GVHD.

GVL Function of BM/S Allografts

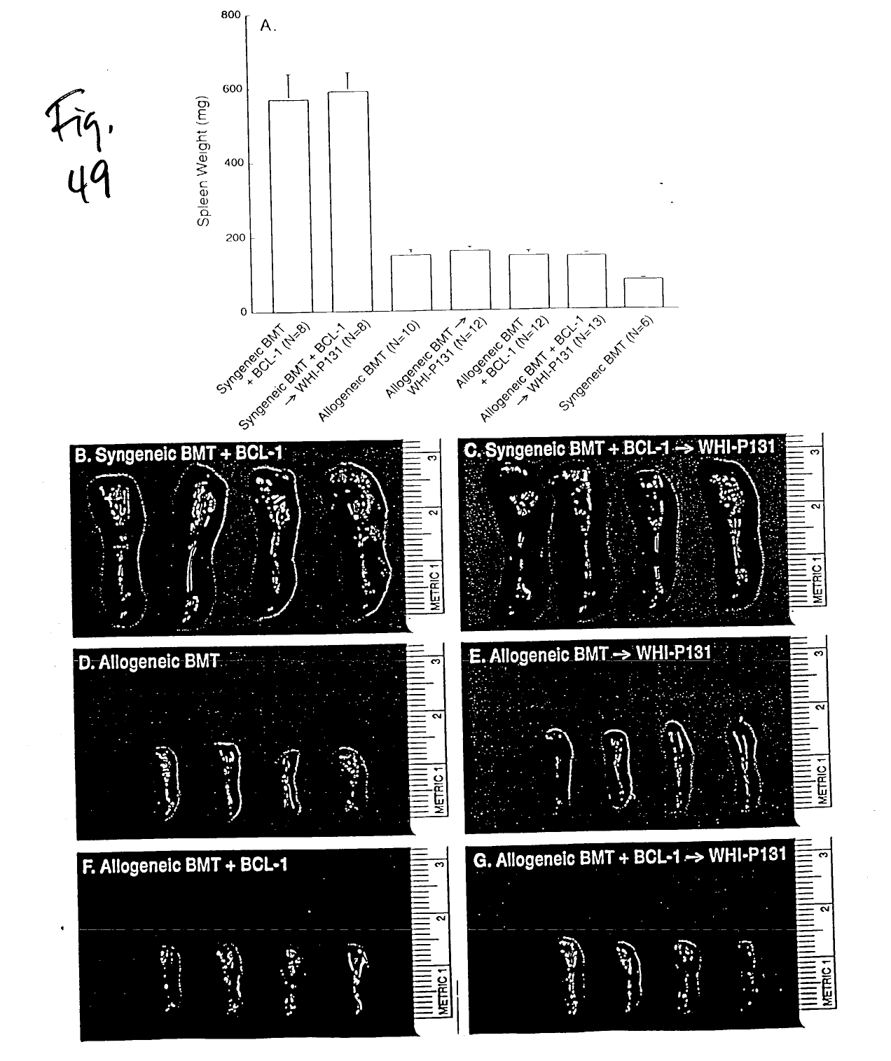

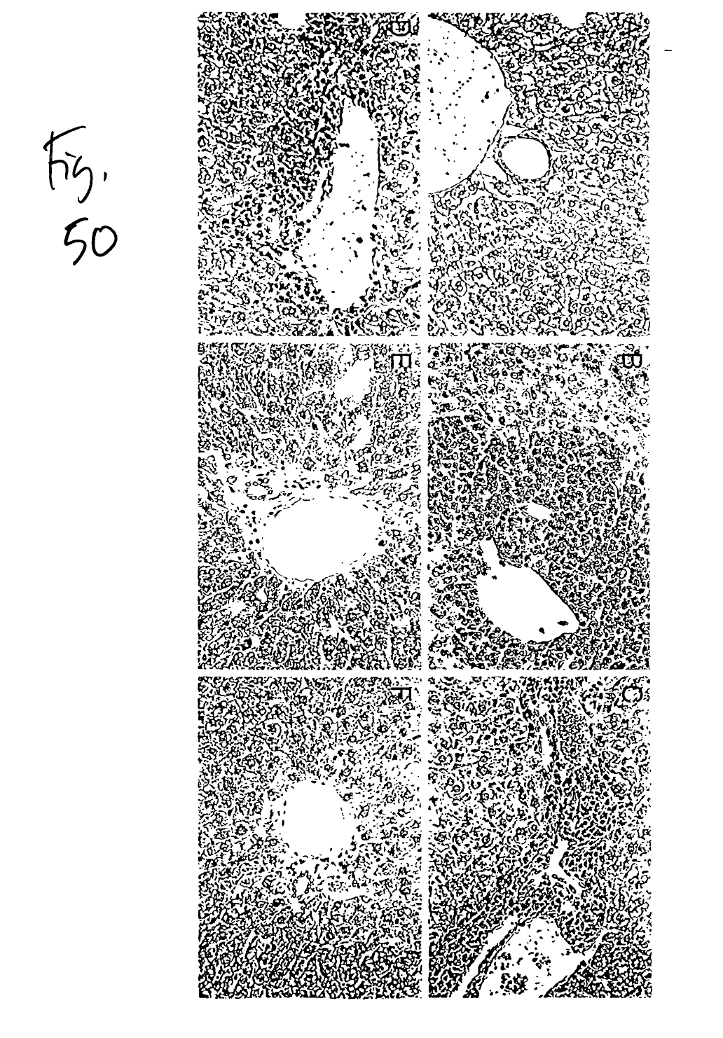

We next sought to determine if BCL-1 cells are sensitive to the antileukemic activity of donor allografts. To this end, F1 mice (H-2^{d}) were first irradiated and then received allogeneic BM/S grafts from C57BL/6 mice (H-2^{b}) or syngeneic BM/S grafts on the same day they received an intravenous inoculum of 5×10⁶ BCL-1 leukemia cells. Mice were electively sacrificed either on day 11 or day 23 post-inoculation of BCL-1 cells and their leukemia burden was assessed by gross as well as histopathologic examination of their spleen and liver, determination of spleen weight, and immunophenotyping of spleen mononuclear cells. Control F1 mice undergoing syngeneic BMT had a small size spleen (76±3 mg) when they were electively sacrificed on day 11. By contrast, control mice undergoing syngeneic BMT and receiving BCL-1 leukemia cells all developed rapidly progressive leukemia and consequently showed massive splenomegaly on day 11 (571±70 mg, P<0.01) (Table 12, FIG. 49). Histopathological examination of the spleen showed diffuse effacement of the normal architecture with leukemic cells. Leukemic infiltrates were easily detectable in the liver as well (FIG. 50B).

Unlike F1 mice undergoing syngeneic BMT, none of the F1 mice undergoing allogeneic BMT that were inoculated with the same number of BCL-1 cells developed leukemia (Table 4, FIG. 49). The average day 11 spleen weights were 176±17 mg for mice undergoing allogeneic BMT without a BCL-1 challenge and 163±11 mg for mice undergoing allogeneic BMT and receiving a BCL-1 inoculum (Table 12). Similarly, the average day 23 spleen weights were 111±10 mg for mice undergoing allogeneic BMT without a BCL-1 challenge and 121±8 mg for mice undergoing allogeneic BMT and receiving a BCL-1 inoculum (Table 12). No leukemic infiltrates were found in the day 11 or day 23 spleens or livers of allotransplanted mice (Table 12, FIG. 50D).

TABLE 12
Leukemia Burden of BMT Recipient Mice Challenged
with BCL-1 Leukemia Cells

| | | Histopathologic | Spleen BCL-1 Leukemia Burden | | |
|---|---|---|---|---|---|
| | | Evidence of | | | |
| | | Leukemia | | | BCL-1 |
| | Time Post | in Spleen or | Spleen | B220⁺ | B220⁺ H-2D^{b-} |
| Group | BMT | Liver | Weight (mg) | (%) | (% of B220⁺) |
| A | d11 | 0/6 | 76 ± 3 (n = 6) | 58 ± 3 (n = 6) | 0 ± 0 (n = 6) |
| B | d11 | 8/8 | 571 ± 70 (n = 8) | 63 ± 8 (n = 4) | 48 ± 13 (n = 4) |
| C | d11 | 8/8 | 591 ± 51 (n = 8) | 44 ± 2 (n = 4) | 40 ± 4 (n = 4) |
| D | d11 | 0/6 | 176 ± 17 (n = 6) | 9 ± 0 (n = 3) | 0 ± 0 (n = 3) |
| | d23 | 0/4 | 111 ± 10 (n = 4) | ND | ND |
| E | d11 | 0/7 | 163 ± 11 (n = 7) | 8 ± 1 (n = 3) | 0 ± 0 (n = 3) |
| | d23 | 0/5 | 121 ± 8 (n = 5) | ND | ND |
| F | d11 | 0/7 | 181 ± 11 (n = 7) | 6 ± 1 (n = 3) | 0 ± 0 (n = 3) |
| | d23 | 0/6 | 102 ± 4 (n = 6) | ND | ND |

The leukemia burden of mice challenged with BCL-1 cells was determined by measuring the spleen weight, histopathologic examination of liver and spleen, and by immunophenotypic analysis of spleen cells, as described above. BCL-1 cells were identified as B220⁺H2-D^{b-} cells. WHI-P131 was used at a dose level of 50 mg/kg/day as described above.

We next used immunophenotyping with a highly sensitive quantitative multiparameter flow cytometric method to examine the spleens of allotransplanted F1 mice for the presence of B220⁺H-2D^{b-} BCL-1 leukemia cells on day 11 post BMT. Whereas 48±13% of the nucleated spleen cell populations from BCL-1 challenged F1 mice undergoing syngeneic BMT were B220⁺H-2D^{b-} BCL-1 cells, no discrete leukemic cell population could be identified in the spleens of allotransplanted F1 mice by flow cytometric immunophenotyping (Table 12). The percentages of B220⁺ B-lineage cells in the spleen cell suspensions were 9.3±0.1% for allotransplanted F1 mice not challenged with BCL-1 leukemia cells and 7.9±0.9% for allotransplanted F1 mice that were challenged with BCL-1 leukemia cells (Table 12). These B220⁺ cells were 100% H-2Db⁺ consistent with their nonleukemic origin. (Table 12).

We next set out to confirm the absence of evidence of residual leukemic cells in allotransplanted F1 recipient mice challenged with BCL-1 cells using F1 to BALB/c adoptive transfer experiments. To this end, 1×10⁶ splenocytes obtained from these F1 recipients either 11 days (N=7) or 23 days (N=5) post BMT/BCL-1 inoculation were injected into the caudal vein of secondary BALB/c recipients. Notably, 8 of 9 BALB/c mice inoculated with day 11 splenocytes and 8 of 8 BALB/c mice inoculated with day 23 splenocytes survived without any evidence of leukemia >100 days and were electively sacrificed on day 101. In contrast, 100% of control BALB/c recipients (N=9) inoculated with day 11 post-BMT splenocytes from F1 mice undergoing syngeneic BMT (N=8) developed fatal leukemia and died with a median survival of 15 days (Table 13). At the time of death, control BALB/c recipients were found to have massive splenomegaly, whereas BALB/c recipients of day 11 or day 23 splenocytes from allotransplanted F1 mice had normal size spleens (1610±70 mg vs 125±6 mg and 123±5 mg, respectively; P<0.001; Table 13). Since 10 BCL-1 cells kill BALB/c mice within 12 weeks, these results demonstrate that the splenocyte suspensions used in the adoptive transfer experiments contained <10/1,000,000 (<0.001%) BCL-1 cells.

TABLE 13:
Detection of BCl-1 Leukemic Cells in Spleen of BMT Recipient Mice
by Adoptive Transfer to Secondary BALB/c Recipient Mice.

| | Donors | | | Recipients | | | |
|---|---|---|---|---|---|---|---|
| | | | | | No. of | Median | pleen Weight |
| | Days Post | | | | Survivors/ | Survival | t the Time of |
| Group | Cl-1/BMT | N | No. Cells | N | Total Mice | Time (days) | eath (mg) |
| A | 11 | 8 | 1 × 10⁶ | 9 | 0/9 | 15 | 1610 ± 70 |
| B | 11 | 7 | 1 × 10⁶ | 9 | 8/9 | >100 | 125 ± 6 |
| | 23 | 5 | 1 × 10⁶ | 8 | 8/8 | >100 | 123 ± 5 |
| C | 11 | 7 | 1 × 10⁶ | 9 | 9/9 | >100 | 122 ± 3 |
| | 23 | 6 | 1 × 10⁶ | 7 | 7/7 | >100 | 137 ± 12 |
| | >60 | 9 | 0.5- | 9 | 9/9 | >100 | 117 ± 6 |
| | | | 1 × 10⁶ | | | | |
| D | 11 | 8 | 1 × 10⁶ | 9 | 0/9 | 15 | 1500 ± 97 |

Splenocytes were obtained at the indicated time points from lethally irradiated F1 mice reconstituted with
(A) syngeneic BM/S grafts and injected with BCL-1 cells,
(B) allogeneic BM/S grafts and injected with BCL-1 cells,
(C) same as group B, but treated with WHI-P131,
(D) same as group A but treated with WHI-P131.

In accordance with these observations, allogeneic BMT changed the survival outcome of FI mice after inoculation of BCL-1 leukemia cells. Unlike F1 mice undergoing syngeneic BMT (N=22) which all developed rapidly progressive and fatal leukemia after inoculation of 5×10⁶ BCL-1 cells with a median survival of 14 days, none of the 28 F1 mice undergoing allogeneic BMT that were inoculated with the same number of BCL-1 cells developed leukemia (Table 9). Furthermore, the survival outcome of allotransplanted F1 mice challenged with BCL-1 leukemia cells (median survival=25 days, N=28) was virtually identical to the survival outcome of allotransplanted control F1 mice that were not inoculated with BCL-1 leukemia cells (median survival=24 days, N=23) (Table 9, FIG. 47 vs FIG. 48).

Allotransplanted F1 mice that were inoculated with BCL-1 cells on the day of their BMT showed no detectable splenomegaly at their postmortem examination and histopathologic examination of their spleen or liver revealed no morphologic evidence of leukemic infiltrates (FIG. 49, Table 12). Thus, the challenge with BCL-1 leukemia cells did not worsen the survival of F1 mice undergoing allogeneic BMT, which is likely due to the GVL function of the BM/S allografts. Taken together, these results provide strong experimental evidence for a potent in vivo GVL function of the allografts from C57BL/6 mice (H-2^{b+}) against BCL-1 (H-2^{b-}) leukemia cells. However, all of these mice eventually developed severe GVHD and died with a median survival of 25 days (Table 9, FIG. 48). The average organ GVHD scores were 2.6±0.1 for the liver, 1.5±0.2 for the skin, 0.8±0.1 for the small intestine and 1.2±0.3 for the large intestine (Table 10).

GVHD Prophylaxis by Targeting Janus Kinase 3 with the Chemical Inhibitor WHI-P131 Spares the GVL Function of the HLA-Disparate Marrow/Spleen Allografts.

We first sought to determine the effect of WHI-P131 on the GVL function of the allografts. WHI-P131 treated allotransplant recipients that were inoculated with BCL-1 cells on the day of their BMT showed no splenomegaly at their postmortem (day 11 or day 23 after inoculation of BCL-1 cells) examination and histopathologic examination of their spleen or liver revealed no morphologic evidence of leukemic infiltrates (FIG. 49, Table 12). Furthermore, no discrete leukemic cell population could be identified in the spleens of WHI-P131 treated allotransplant recipients by flow cytometric immunophenotyping (Table 12). The percentage of B220⁺ B-lineage cells in the spleen cell suspensions was 6±1% and all of these B220⁺ cells were 100% H-2D^{b+} consistent with their nonleukemic origin. (Table 12). The absence of leukemia in WHI-P131-treated allotransplant recipient mice was due to the GVL function of the allografts rather than an antileukemic effect of WHI-P131 since WHI-P131 treatment of BCL-1-challenged F1 mice undergoing syngeneic BMT did not prevent the development of leukemia and leukemia-associated splenomegaly (Table 12, FIG. 49). Thus, treatment with WHI-P131 did not abolish the GVL function of the allografts.

We next performed adoptive transfer experiments to confirm the absence of residual leukemic cells in WHI-P131 treated allotransplant recipients challenged with BCL-1 cells. To this end, 1×10⁶ splenocytes obtained from WHI-P131-treated F1 recipients either 11 days (N=7), 23 days (N=6) or >60 days (N=9) post BMT/BCL-1 inoculation were injected into the caudal vein of secondary BALB/c recipients. Notably, all of the BALB/c mice inoculated with splenocytes from WHI-P131-treated allotransplant recipients survived >100 days without any evidence of leukemia. In contrast, 100% of control BALB/c recipients (N=9) inoculated with day 11 post-BMT splenocytes from WHI-P131 treated F1 mice undergoing syngeneic BMT (N=8) developed fatal leukemia and died with a median survival of 15 days (Table 13). At the time of death, control BALB/c recipients had massive splenomegaly whereas BALB/c recipients of splenocytes from allotransplanted F1 mice did not (1500±97 mg vs 122±3 mg and 137±12 mg and 117±6 mg, respectively; P<0.001; Table 13). Since 10 BCL-1 cells kill BALB/c mice within 12 weeks, these results demonstrate that the splenocyte suspensions used in the adoptive transfer experiments contained <10/1,000,000 (<0.001%) BCL-1 cells.

We then set out to determine if the survival outcome of allotransplanted F1 recipients challenged with BCL-1 leukemia cells could be improved by attenuating the severity of GVHD with the JAK3 inhibitor WHI-P131. Recipient mice were treated from the day of BMT/BCL-1 inoculation on with WHI-P131 (25 mg/kg/dose, 2×/day, i.p. injection) (N=16) or vehicle (N=28). As shown in FIG. 48, GVHD prophylaxis using WHI-P131 markedly improved the post-BMT survival outcome. The probability of survival at 30 days post-BMT was 11±6% for vehicle-treated recipients (median survival time=25 days) versus 63±12% for recipients treated with WHI-P131 (median survival time=36 days) (P<0.0001, Table 9; FIG. 48). Since WHI-P131 is devoid of antileukemic activity against BCL-1 leukemia cells (see Table 1 and FIG. 46), this marked improvement in survival outcome was due to reduced incidence of GVHD-associated fatalities in the WHI-P131 group. All of the syngeneic BMT recipients treated with WHI-P131 died of leukemia with a median survival time of 12 days after the BCL-1 challenge (Table 9, FIG. 46B). Because of the sustained GVL function of the allografts, the challenge with BCL-1 leukemia cells did not worsen the post-BMT survival outcome of WHI-P131 treated allograft recipients (Table 9).

The longterm survivors in the WHI-P131 treated group showed no clinical signs of GVHD or leukemia (FIG. 50F). According to the GVHD scoring system, the average GVHD grades were 0.6±0.1 for the liver, 0.0±0.0 for the skin, 0.3±0.1 for the small intestine and 0.1±0.1 for the large intestine (Table 2). Flow cytometric H-2D^{d/b}-typing of splenocytes obtained from these four long-term survivors of WHI-P131-treated group showed 95.3±0.8% H-2D^{d-/b+} donor cell engraftment (Table 11), indicating that WHI-P131 does not prevent donor cell engraftment under these experimental conditions and the attenuation of GVHD in WHI-P131-treated recipient mice was not due to lack of donor cell engraftment with concomitant autologous recovery.

Severe graft-versus-host-disease (GVHD) hampers a successful outcome of allogeneic BMT in leukemia patients. Cytotoxic T-cells in the donor marrow grafts play a pivotal role in the pathogenesis of GVHD. Cytotoxic donor T-cells capable of recognizing alloantigens and/or leukemia-associated antigens on the surface of host leukemic cells are also thought to be the key effectors of the graft-versus-leukemia (GVL) function of marrow allografts. Since contemporary methods for GVHD prophylaxis, including ex vivo T-cell depletion of marrow grafts, use of positively selected CD34⁺ hematopoietic precursor cells, and systemic immunosuppression are associated with an increased risk of relapse in leukemia patients undergoing BMT, there is an urgent need for novel strategies for GVHD prophylaxis which spare the GVL function of the marrow allografts.

Several lines of evidence have suggested that the GVL function of the allogeneic marrow grafts can be separated from their GVHD activity. Most recently, Schmaltz et al. elegantly demonstrated in an experimental murine BMT model that donor T-cells make differential use of cytotoxic pathways and inhibition of a specific pathway may permit prevention of GVHD without any loss of the GVL function. Specifically, these investigators found that the Fas ligand pathway is responsible for the GVHD (but not GVL) function of the alloreactive donor T-cells. In another recent study, Patterson and Korngold discovered in a murine BMT model that infusion of selectively expanded leukemia-reactive T cell receptor (TCR) Vbeta+ T cells identified by complementarity-determining region 3 (CDR3)-size spectratyping provides GVL responses with negligible GVHD. (Patterson AI, Korngold R. Infusion of select leukemia-reactive TCR Vbeta+ T cells provides graft-versus-leukemia responses with minimization of graft-versus-host disease following murine hematopoietic stem cell transplantation. Biol Blood Marrow Transplant. 2001; 7:187-196).

Signal transducers and activators of transcription (STAT) are pleiotropic transcription factors which mediate cytokine-stimulated gene expression in multiple cell populations. All STAT proteins contain a DNA binding domain, a Src homology 2 (SH2) domain, and a transactivation domain necessary for transcriptional activation of target gene expression. Janus kinases (JAK), including JAK1, JAK2, Tyk, and JAK3, are cytoplasmic protein tyrosine kinases (PTK) which play pivotal roles in initiation of cytokine-triggered signaling events by activating the cytoplasmic latent forms of STAT proteins via tyrosine phosphorylation on a specific tyrosine residue near the SH2 domain. Tyrosine phosphorylated STAT proteins dimerize through specific reciprocal SH2-phosphotyrosine interactions and translocate from the cytoplasm to the nucleus where they stimulate the transcription of specific target genes by binding to response elements in their promoters. Among the four members of the JAK family, JAK3 is abundantly expressed in lymphoid cells and plays an important role in normal lymphocyte development and function, as evidenced by qualitative and quantitative deficiencies in the B-cell as well as T-cell compartments of the immune system of JAK3-deficient mice and development of severe combined immunodeficiency in JAK3-deficient patients. JAK3 is expressed at very high levels in human lymphoblastic as well as myeloblastic leukemia cells and several studies have correlated constitutive STAT activation in human leukemia cells with resistance to apoptosis-inducing chemotherapeutic agents.

We recently reported the structure-based design of specific inhibitors of Janus kinase 3 (JAK3) as apoptosis-inducing antileukemic agents. The lead compound 4-(4′-hydroxyphenyl)-amino-6,7-dimethoxyquinazoline (WHI-P131) inhibited JAK3 but not the other Janus kinases JAK1 or JAK2. Similarly, the ZAP/SYK family tyrosine kinase SYK, TEC family tyrosine kinase BTK, SRC family tyrosine kinase LYN, and receptor family tyrosine kinase IRK were not inhibited by WHI-P131. The use of this compound in biological assays confirmed that JAK3 is a vital target in human leukemia cells and demonstrated that WHI-P131 triggers apoptosis in lymphoblastic as well as myeloblastic leukemia cells. WHI-P131 was very well tolerated by mice and monkeys and plasma concentrations of WHI-P131 that are cytotoxic to human leukemia cells in vitro could be achieved at non-toxic dose levels. The antileukemic activity and lack of significant systemic toxicity of WHI-P131 suggest that this JAK3 inhibitor may be useful in the treatment of relapsed or therapy-refractory leukemia patients. Intriguingly, WHI-P131 did not cause any myelosuppression in mice or monkeys, indicating that JAK3 does not have a critical non-redundant function in normal myelopoiesis.

The pivotal role of JAK3 in normal lymphocyte development and function along with the cytotoxic effects of WHI-P131 on leukemia cells prompted the hypothesis that this JAK3 inhibitor could be useful as a dual-function anti-GVHD agent with potent anti-leukemic activity. In a more recent study, we examined the effectiveness of targeting JAK3 with WHI-P131 for prevention and treatment of lethal GVHD across major histocompatibility barrier in mice. WHI-P131 exhibited potent in vivo biologic activity in an aggressive acute GVHD model using BALB/c (H-2^{d}) donor bone marrow/spleen cells and H-2 disparate C57BL/6 (H-2^{b}) recipient mice. WHI-P131 could improve the survival outcome even when the therapy was delayed until the 3rd week after BMT.

One of the purpose of the present study was to evaluate the effects of GVHD prophylaxis with the JAK3 inhibitor WHI-P131 on the GVL function of marrow allografts in mice undergoing BMT after being challenged with an otherwise invariably fatal dose of BCL-1 leukemia cells. We first confirmed that (a) severe GHVD can be induced in lethally irradiated (BALB/cJxC57BL/6J) F1 mice (H-2^{d/b}) across the MHC barrier by injection of BM/S grafts from C57BL/6 mice (H-2^{b}) and (b) WHI-P131 can improve survival in this BMT model by attenuating the severity of GVHD. We next set out to characterize the in vivo GVL function of the allografts from C57BL/6 mice (H-2^{b+}) against BCL-1 (H-2^{b-}) leukemia cells by comparing the BMT survival outcome of syngeneic versus allogeneic BMT recipients. Detailed studies using pathologic and histopathologic examinations, immunophenotyping, and adoptive transfer experiments aimed at identifying residual BCL-1 leukemia cells in allotransplanted F1 recipients provided strong experimental evidence for a potent in vivo GVL function of the allografts from C57BL/6 mice (H-2^{b+}) against BCL-1 (H-2^{b-}) leukemia cells.

We next set out to determine if the survival outcome of allotransplanted F1 recipients challenged with BCL-1 leukemia cells could be improved by attenuating the severity of GVHD with the JAK3 inhibitor WHI-P131. Notably, GVHD prophylaxis using WHI-P131 markedly improved the post-BMT survival outcome. Since WHI-P131 is devoid of antileukemic activity against BCL-1 leukemia cells, the improvement in survival outcome was due to reduced incidence of GVHD-associated fatalities combined with sustained GVL function of the allografts in the WHI-P131 group. The longterm survivors in the WHI-P131 treated group showed no clinical signs of GVHD or leukemia. Notably, adoptive transfer experiments demonstrated that the spleens of WHI-P131 treated allograft recipients contained <0.001% BCL-1 cells.

Taken together, our results provide strong experimental evidence that GVHD prophylaxis using a JAK3 inhibitor such as WHI-P131 does not impair the GVL function of the allografts and consequently contributes to an improved post-BMT survival outcome of the recipient mice. In the present study, we used the JAK3-negative BCL-1 leukemia cells in order to avoid the anti-leukemic activity of WHI-P131 against JAK3-positive leukemia cells as a confounding factor in the evaluation of its effects on the GVL function of marrow allografts. We hypothesize that the antileukemic activity of WHI-PI 31 against JAK3-expressing human leukemia cells ¹⁸ may further enhance its ability to attenuate the severity of GVHD without increasing the risk of relapse post-BMT in clinical settings.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of treating graft versus host disease in a mammal comprising:
administering to a mammal a graft versus host disease treating effective amount of;
(a) methotrexate; and
(b) a compound of formula I: 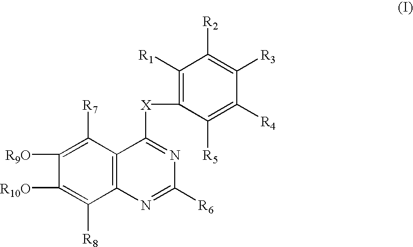
wherein
X is selected from the group consisting of HN, R₁₁N, S, O, CH₂, and R₁₁CH;
R₁₁ is (C₁-C₄)alkyl or (C₁-C₄)alkanoyl;
R₁-R₅ are each independently selected from the group consisting of hydrogen, hydroxy and halo;
R₆, R₇, and R₈ are each independently selected from the group consisting of hydrogen, hydroxy, mercapto, amino, nitro, (C₁-C₄)alkyl, (C₁-C₄)alkoxy, (C₁-C₄)alkylthio and halo; and
R₉ and R₁₀ are each independently hydrogen, (C₁-C₄)alkyl, (C₁-C₄)alkoxy, halo or (C₁-C₄)alkanoyl; or R₉ and R₁₀ together are methylenedioxy; or a pharmaceutically acceptable salt thereof.

2. The method of claim 1 wherein the compound is 4-(3-hydroxyl-phenyl)-amino-6,7-dimethoxyquinazoline; 4-(3′,5′-dibromo-4-hydroxyl-phenyl)-amino-6,7-dimethoxyquinazoline or 4-(3′-bromo-4-hydroxyl-phenyl)-amino-6,7-dimethoxyquinazoline; or a pharmaceutically acceptable salt thereof.

3. The method of claim 1 wherein the compound is 4-(4-hydroxyl-phenyl)-amino-6,7-dimethoxyquinazoline; or a pharmaceutically acceptable salt thereof.

4. The method of claim 1, wherein the compound is administered at a dosage of about 0.5 mg/kg/day to about 100 mg/kg/day.

5. The method of claim 1, wherein the administration of the compound of formula I does not compromise the graft-versus-leukemia function of the bone marrow transplant graft.

6. A method comprising;
administering to a mammal a graft versus host disease treating effective amount of;
(a) methotrexate; and
(b) a compound of formula II: 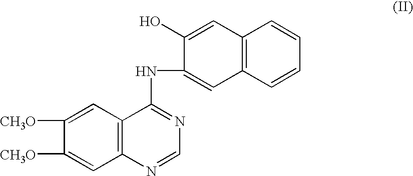
or a pharmaceutically acceptable salt thereof.

7. A method for prophylactically treating a mammal to prevent graft versus host disease comprising:
administering to a mammal a graft versus host disease prophylactically effective amount of a compound of formula I: 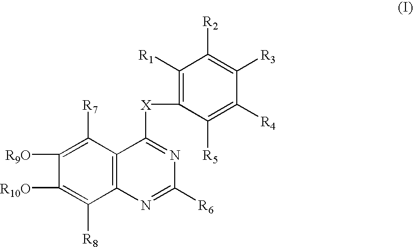
wherein
X is selected from the group consisting of HN, R₁₁N, S, O, CH₂, and R₁₁CH;
R₁₁ is (C₁-C₄)alkyl or (C₁-C₄)alkanoyl;
R₁-R₅ are each independently selected from the group consisting of hydrogen, hydroxy and halo;
R₆, R₇, and R₈ are each independently selected from the group consisting of hydrogen, hydroxy, mercapto, amino, nitro, (C₁-C₄)alkyl, (C₁-C₄)alkoxy, (C₁-C₄)alkylthio and halo; and
R₉ and R₁₀ are each independently hydrogen, (C₁-C₄)alkyl, (C₁-C₄)alkoxy, halo or (C₁-C₄)alkanoyl; or R₉ and R₁₀ together are methylenedioxy; or a
pharmaceutically acceptable salt thereof.

8. The method of claim 7, wherein the compound is 4-(3-hydroxyl-phenyl)-amino-6,7-dimethoxyquinazoline; 4-(3′,5′-dibromo-4-hydroxyl-phenyl)-amino-6,7-dimethoxyquinazoline or 4-(3′-bromo-4-hydroxyl-phenyl)-amino-6,7-dimethoxyquinazoline; or a pharmaceutically acceptable salt thereof.

9. The method of claim 7 wherein the compound is 4-(4-hydroxyl-phenyl)-amino-6,7-dimethoxyquinazoline; or a pharmaceutically acceptable salt thereof.

10. The method of claim 7, wherein the compound is administered at a dosage of about 0.5 mg/kg/day to about 100 mg/kg/day.

11. The method of claim 7, further comprising administering methotrexate.

12. A composition comprising:
(a) a therapeutically effective amount of methotrexate; and
(b) a therapeutically effective amount of a compound of formula I: 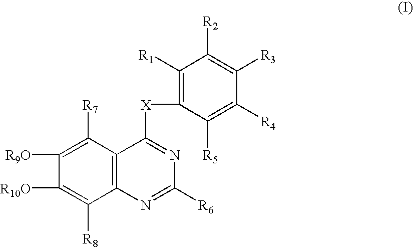
wherein
X is selected from the group consisting of HN, R₁₁N, S, O, CH₂, and R₁₁CH;
R₁₁ is (C₁-C₄)alkyl or (C₁-C₄)alkanoyl;
R₁-R₅ are each independently selected from the group consisting of hydrogen, hydroxy and halo;
R₆, R₇, and R₈ are each independently selected from the group consisting of hydrogen, hydroxy, mercapto, amino, nitro, (C₁-C₄)alkyl, (C₁-C₄)alkoxy, (C₁-C₄)alkylthio and halo; and
R₉ and R₁₀ are each independently hydrogen, (C₁-C₄)alkyl, (C₁-C₄)alkoxy, halo or (C₁-C₄)alkanoyl; or R₉ and R₁₀ together are methylenedioxy; or a pharmaceutically acceptable salt thereof.

13. The composition of claim 12 wherein the compound is 4-(3-hydroxyl-phenyl)-amino-6,7-dimethoxyquinazoline; 4-(3′,5′-dibromo-4-hydroxyl-phenyl)-amino-6,7-dimethoxyquinazoline or 4-(3′-bromo-4-hydroxyl-phenyl)-amino-6,7-dimethoxyquinazoline; or a pharmaceutically acceptable salt thereof.

14. The composition of claim 12 wherein the compound is 4-(4-hydroxyl-phenyl)-amino-6,7-dimethoxyquinazoline; or a pharmaceutically acceptable salt thereof.

15. The composition of claim 12, wherein methotrexate and the compound of formula I are present in amounts therapeutically effective to treat graft versus host disease.

16. The composition of claim 12, wherein methotrexate and the compound of formula I are present in amounts therapeutically effective to prevent graft versus host disease.

17. A composition comprising;
(a) a therapeutically effective amount of methotrexate; and
(b) a therapeutically effective amount of a compound of formula II: 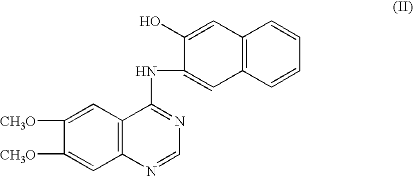
or a pharmaceutically acceptable salt thereof.

18. The composition of claim 17, wherein methotrexate and the compound of formula I are present in amounts therapeutically effective to treat graft versus host disease.

19. The composition of claim 17, wherein methotrexate and the compound of formula I are present in amounts therapeutically effective to prevent graft versus host disease.

20. A pharmaceutical composition comprising:
(a) a therapeutically effective amount of methotrexate;
(b) a therapeutically effective amount of a compound of formula I: 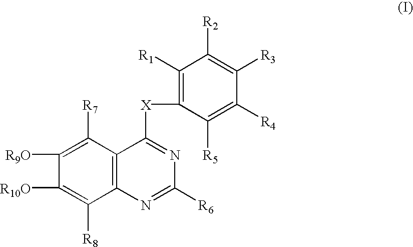
wherein
X is selected from the group consisting of HN, R₁₁N, S, O, CH₂, and R₁₁CH;
R₁₁ is (C₁-C₄)alkyl or (C₁-C₄)alkanoyl;
R₁-R₅ are each independently selected from the group consisting of hydrogen, hydroxy and halo;
R₆, R₇, and R₈ are each independently selected from the group consisting of hydrogen, hydroxy, mercapto, amino, nitro, (C₁-C₄)alkyl, (C₁-C₄)alkoxy, (C₁-C₄)alkylthio and halo; and
R₉ and R₁₀ are each independently hydrogen, (C₁-C₄)alkyl, (C₁-C₄)alkoxy, halo or (C₁-C₄)alkanoyl; or R₉ and R₁₀ together are methylenedioxy; or a pharmaceutically acceptable salt thereof; and
(c) a pharmaceutically acceptable carrier.

21. The pharmaceutical composition of claim 20 wherein the compound is 4-(3-hydroxyl-phenyl)-amino-6,7-dimethoxyquinazoline; 4-(3′,5′-dibromo-4-hydroxyl-phenyl)-amino-6,7-dimethoxyquinazoline or 4-(3′-bromo-4-hydroxyl-phenyl)-amino-6,7-dimethoxyquinazoline; or a pharmaceutically acceptable salt thereof.

22. The pharmaceutical composition of claim 20 wherein the compound is 4-(4-hydroxyl-phenyl)-amino-6,7-dimethoxyquinazoline; or a pharmaceutically acceptable salt thereof.

23. The pharmaceutical composition of claim 20, wherein methotrexate and the compound of formula I are present in amounts therapeutically effective to treat graft versus host disease.

24. The pharmaceutical composition of claim 20, wherein methotrexate and the compound of formula I are present in amounts therapeutically effective to prevent graft versus host disease.

25. A pharmaceutical composition comprising;
(a) a therapeutically effective amount of methotrexate; and
(b) a therapeutically effective amount of a compound of formula II: 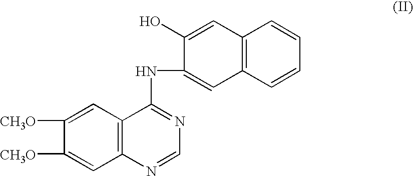
or a pharmaceutically acceptable salt thereof; and
(c) a pharmaceutically acceptable carrier.

26. The pharmaceutical composition of claim 25, wherein methotrexate and the compound of formula II are present in amounts therapeutically effective to treat graft versus host disease.

27. The pharmaceutical composition of claim 25, wherein methotrexate and the compound of formula II are present in amounts therapeutically effective to prevent graft versus host disease.
